SEQUENCE LISTING (13 sequences)

SEQ ID NO: 1 - DNA, 2451 bp, Homo sapiens
agaggagtgtttagctccttcccttactctaccttgctcctacttttctctaagtcaacatgagtcgacagtttagttccaggtctgggtaccgaagtggagggggcttcagctctggctctgctgggatcatcaactaccagcgcaggaccaccagcagctccacacgccgcagtggaggaggtggtgggagattttcaagctgtggtggtggtggtggtagctttggtgctggtggtggatttggaagtcggagtcttgttaaccttggtggcagtaaaagcatctccataagtgtggctagaggaggtggacgtggtagtggctttggtggtggttatggtggtggtggctttggtggtggtggctttggtggtggtggctttggtggaggtggcattgggggtggtggctttggtggttttggcagtggtggtggtggttttggtggaggtggctttgggggtggtggatatgggggtggttatggtcctgtctgccctcctggtggcatacaagaagtcactatcaaccagagccttcttcagcccctcaatgtggagattgaccctgagatccaaaaggtgaagtctcgagaaagggagcaaatcaagtcactcaacaaccaatttgcctccttcattgacaaggtgaggttcctggagcagcagaaccaggtactgcaaacaaaatgggagctgctgcagcaggtagatacctccactagaacccataatttagagccctactttgagtcattcatcaacaatctccgaaggagagtggaccaactgaagagtgatcaatctcggttggattcggaactgaagaacatgcaggacatggtggaggattaccggaacaagtatgaggatgaaatcaacaagcggacaaatgcagagaatgaatttgtgaccatcaagaaggatgtggatggtgcttatatgaccaaggtggaccttcaggccaaacttgacaacttgcagcaggaaattgatttccttacagcactctaccaagcagagttgtctcagatgcagactcaaatcagtgaaactaatgtcatcctctctatggacaacaaccgcagtctcgacctggacagcatcattgctgaggtcaaggcccagtacgaggatatagcccagaagagcaaagctgaggccgagtccttgtaccagagcaagtatgaagagctgcagatcactgctggcagacatggggatagtgtgagaaattcaaagatagaaatttctgagctgaatcgtgtgatccagagacttagatctgaaatcgacaatgtcaagaagcagatctccaacttgcagcagtccatcagtgatgcagagcagcgtggcgagaatgccctcaaggatgccaagaacaagctgaatgacctggaggatgccctgcagcaggccaaggaagacctggcccgcctgctgcgcgactaccaggagctgatgaacacaaagctggccctggatctggagattgccacctacaggaccctcctggagggagaagaaagcaggatgtctggagaatgtgccccgaacgtgagtgtgtctgtgagcacaagccacaccaccatcagtggaggtggcagccgaggaggtggcggcggtggctacggctctggaggtagcagctatggctccggaggtggtagctatggttctggaggtggcggcggcggcggccgtggcagctatggctccggaggtagcagctacggctccggaggtggcagctatggctctggaggtggcggcggcggccatggcagctacggctccggaagcagcagtgggggctacagaggtggctctggaggcggcggcggcggcagctctggcggccggggctctggcggcgggagctctggaggctccataggaggccggggatccagctctgggggtgtcaagtcctctggtggcagttccagcgtgaagtttgtttctaccacttattccggagtaaccagataaagagatgccctctgtttcattagctctagttctcccccagcatcactaacaaatatgcttggcaagaccgaggtcgatttgtcccagccttaccggagaaaagagctatggttagttacactagctcatcctattcccccagctctttcttttctgctgtttcccaatgaagttttcagatcagtggcaatctcagtcccctggctatgaccctgctttgttctttccctgagaaacagttcagcagtgaccaccacccacatgacatttcaaagcacctccttaagccagccagagtaggaccagttagacccagggtgtggacagctccttagcatcttatctctgtgctgttttggttttgtacataaggtgtaagcaagttgtttttcttttgtggagaggtcttaaactccccatttccttgttttgctgcaataaactgcatttgaaattc

SEQ ID NO: 2 - DNA, 4632 bp, Homo sapiens
agggcgaggctgagggcagagagttggacacaaccctgagatttatccctcacaatgcggaaagacagacttcttcatttatgtcttgtgctacttcttatcctgctttctgccagtgactcaaactctacagaaccgcagtatatggtgctggtcccctccctgctccacactgaggcccctaagaagggctgtgtccttctgagccacctgaatgagacagtgactgtaagtgcttccttggagtctggcagggaaaacaggagcctcttcactgacctggtggcggagaaggacttattccactgtgtctccttcactctcccaaggatctcagcctcttcagaggtggcattccttagcatccagataaaggggcctacgcaagatttcaggaagaggaacacagttctggtactgaacacccaaagtctggtctttgtccagacagacaaacccatgtataaaccaggacagacagtaagattccgtgttgtctccgtggatgaaaattttcgccctcgaaatgaactgattccactgatataccttgagaacccaagaagaaatcgaattgcacaatggcagagtctcaagctagaagctggcatcaatcagttgtcctttcccctctcatcagagcccattcagggctcctacagggtggtggtacagacagaatcaggtggaaggatacagcaccccttcaccgtggaggaatttgtgcttcccaagtttgaggtcaaagttcaggtgccaaagataatcagtatcatggatgaaaaagtgaacataacagtctgtggagaatacacttatgggaagcctgtcccaggacttgcaactgtgagcctgtgtagaaaattatctcgtgttcttaattgtgacaagcaggaggtctgtgaggaattcagtcaacagcttaacagcaatggctgcatcacccaacaagtacacaccaaaatgctccagattacaaatacgggctttgaaatgaagcttagagtggaagccaggatcagagaagaggggacagacctggaagtcactgcaaacaggatcagtgaaatcacaaacattgtatccaaactcaaattcgtgaaagtggattcacactttagacaaggaatccccttttttgcacaggtgcttctggtggatggaaaaggtgtgcccatccccaataaactcttcttcatctctgtgaatgacgccaattattactccaatgcaaccaccaatgagcagggtcttgcacagttttcaatcaatactaccagtatctcggttaataaactttttgtccgggttttcactgtgcatcccaacttgtgttttcactattcatgggtagcagaagaccaccagggtgctcagcacactgcaaatcgtgttttctccttaagtggaagttacattcacctggagcctgtggctggtaccctgccctgtggccacacggagactatcacggcacactatacactgaatagacaggccatgggagagttatcggagctcagtttccattacctgatcatggctaagggagtcatcgtcagatctggaacccacactctgcctgtggagtcaggagacatgaaaggcagttttgccttatccttccctgtggagtcagacgttgcccccattgcacgaatgttcatctttgccattttaccagatggagaagttgttggagactctgaaaaatttgagattgaaaactgtctagccaacaaggtggatttgagcttcagcccagcacaaagtcccccagcctcacatgcccacctgcaagtagcagctgctccgcagtccctctgtgcccttcgtgctgtggaccaaagtgtgctgctcatgaagcctgaggctgagctctctgtgtcctcagtatataatctgctaactgtgaaggatctcaccaattttcctgacaatgtggaccagcaggaggaagaacaaggacactgtccccgtcctttcttcattcataatggagccatctatgttcccttatcaagtaatgaagcagatatttatagcttcctcaaggggatgggattgaaggtgttcactaactcaaaaatccgaaaaccaaagtcgtgttcagtcatcccttccgtgtctgcaggagcagtaggtcaaggatactatggagcaggtctaggagtagtagagagaccatatgttcctcaattaggcacatataatgtgatacccttaaataatgaacaaagttcagggccagtccctgaaacggtgcgaagctattttcctgagacttggatctgggagttggtggcagtgaactcatcaggtgtggctgaggtaggagtaacagtccctgacaccatcaccgagtggaaggcaggggccttctgcctgtccgaagatgctggacttggtatctcttccactgcctctctccgagccttccagcccttctttgtggagctcacaatgccttactctgtgattcgtggagaggtcttcacactcaaggccacggtcctaaactaccttcccaaatgcatccgggtcagtgtgcagctgaaagcctctccagccttcctagcttcccaaaatacaaagggagaagaatcctattgtatctgtggaaatgagagacaaaccttgtcttggacagtgactcctaaaactctggggaatgtgaacttctcagtgagtgcagaggcaatgcagtccttagaactctgtggaaatgaggttgttgaggtccctgagattaaaagaaaagacacagtcatcaaaaccctgttggtggaggctgaaggtattgagcaagaaaagactttcagttctatgacctgtgcctcaggtgctaatgtgtctgagcagttgtccttgaagctcccatcaaatgtggtcaaagaatctgccagagcttctttctcagttctgggtgacatattaggttctgctatgcaaaatatacaaaatctcctccagatgccatatggctgtggagaacagaacatggtcctatttgctcctaacatctatgtcttgaactatctgaatgaaacccagcagctgacgcaggagatcaaggccaaggccgttggctatctcatcactggttaccagagacagctgaactacaaacaccaagatggctcctacagcacctttggggaacgatatggcaggaaccagggcaacacttggctcacagcttttgtactgaagactttcgcccaggctcgatcctacatcttcattgatgaagcacacattacccaatctctcacgtggctctcccagatgcagaaggacaatggctgtttcaggagctctgggtcactgctcaacaatgccataaagggaggtgtagaagatgaagcgaccctctccgcctatgttactattgcccttctggaaattcctctcccagtcactaaccctattgttcgcaatgccctgttctgcctggagtcagcctggaatgtagcaaaggaggggacccatgggagccatgtctacaccaaggcattgctggcctatgctttttccctactgggaaagcaaaatcagaatagagaaatactgaactcacttgataaggaagctgtgaaagaagacaacctcgtccattgggagcgccctcagagacccaaggcaccagtggggcatctttaccaaacccaggctccctctgctgaggtggagatgacatcctatgtgctcctcgcttatctcacggcccagccagcccccacctcaggggacctgacctctgcaactaacattgtgaagtggatcatgaagcagcagaacgcccaaggtggtttctcctccacccaggacacagtggtggctctccatgccctgtccaggtatggagcagccactttcaccagaactgagaaaactgcacaggtcaccgttcaggattcacagaccttttctacaaatttccaagtagacaacaacaacctcctattactgcagcagatctcattgccagagctccctggagaatatgtcataacagtaactggggaaagatgtgtgtatcttcagacatccatgaaatacaatattcttccagagaaagaggactccccatttgctttaaaagtgcagactgtgccccaaacttgcgatggacacaaagcccacaccagctttcagatctcactgaccatcagttacacaggaaaccgtcctgcttccaatatggtgattgttgatgtaaagatggtatctggttttattcccctgaaaccaacagtaaaaatgcttgaaagatctagctctgtgagccggacagaagtgagcaacaaccatgtcctcatttatgtggaacaggtgacaaatcagacgctaagtttttccttcatggttctgcaagacatcccagtaggagacttgaagccagcaattgttaaagtctatgattactatgagacagatgagtctgtggttgctgagtatatcgccccctgcagcacagatacagagcatggaaatgtttgaggaccatacaggctgtatattttggtggattctctgtcctatacatttacttagaaggaatggagttatttgtctctataaaatagacactaaaaatatttgctgaataaatatgtactggtcaaacta

SEQ ID NO: 3 - DNA, 1636 bp, Homo sapiens
acccgagcaccttctcttcactcagccaactgctcgctcgctcacctccctcctctgcaccatgaccacctgcagccgccagttcacctcctccagctccatgaagggctcctgcggcatcgggggcggcatcgggggcggctccagccgcatctcctccgtcctggccggagggtcctgccgcgcccccagcacctacgggggcggcctgtctgtctcatcctcccgcttctcctctgggggagcctgcgggctggggggcggctatggcggtggcttcagcagcagcagcagcagctttggtagtggctttgggggaggatatggtggtggccttggtgctggcttgggtggtggctttggtggtggctttgctggtggtgatgggcttctggtgggcagtgagaaggtgaccatgcagaacctcaatgaccgcctggcctcctacctggacaaggtgcgtgctctggaggaggccaacgccgacctggaagtgaagatccgtgactggtaccagaggcagcggcctgctgagatcaaagactacagtccctacttcaagaccattgaggacctgaggaacaagattctcacagccacagtggacaatgccaatgtccttctgcagattgacaatgcccgtctggccgcggatgacttccgcaccaagtatgagacagagttgaacctgcgcatgagtgtggaagccgacatcaatggcctgcgcagggtgctggacgaactgaccctggccagagctgacctggagatgcagattgagagcctgaaggaggagctggcctacctgaagaagaaccacgaggaggagatgaatgccctgagaggccaggtgggtggagatgtcaatgtggagatggacgctgcacctggcgtggacctgagccgcattctgaacgagatgcgtgaccagtatgagaagatggcagagaagaaccgcaaggatgccgaggaatggttcttcaccaagacagaggagctgaaccgcgaggtggccaccaacagcgagctggtgcagagcggcaagagcgagatctcggagctccggcgcaccatgcagaacctggagattgagctgcagtcccagctcagcatgaaagcatccctggagaacagcctggaggagaccaaaggtcgctactgcatgcagctggcccagatccaggagatgattggcagcgtggaggagcagctggcccagctccgctgcgagatggagcagcagaaccaggagtacaagatcctgctggacgtgaagacgcggctggagcaggagatcgccacctaccgccgcctgctggagggcgaggacgcccacctctcctcctcccagttctcctctggatcgcagtcatccagagatgtgacctcctccagccgccaaatccgcaccaaggtcatggatgtgcacgatggcaaggtggtgtccacccacgagcaggtccttcgcaccaagaactgaggctgcccagccccgctcaggcctaggaggccccccgtgtggacacagatcccactggaagatcccctctcctgcccaagcacttcacagctggaccctgcttcaccctcaccccctcctggcaatcaatacagcttcattatctgagttgcataa

SEQ ID NO: 4 - DNA, 3766 bp, Homo sapiens
cccagccaggacatggccgcacctctcctcatcaggagcgccggctcacggacttctcgcccaactccctgagcgctccctcgtttcgatctttagaaaaccctgctttctttctggggccgtgacgaggggcagggagcggcgagcaaggatgcgttgaggaccgcgagggcgcgcgtctcgggtgccgccgtgggtcccgacgcggaagccgagccgcctccgcctgcctcgacttccccacagcgcttccgccgccgcctgccgtgcttgatgtgcagaaagaagccggacaccatgatcctaacacaaattgaagccaaggaagcttgtgattggctacgggcaactggtttcccccagtatgcacagctttatgaagatttcctgttccccatcgatatttccttggtcaagagagagcatgattttttggacagagatgccattgaggctctatgcaggcgtctaaatactttaaacaaatgtgcagtgatgaagctagaaattagtcctcatcggaaacgaagtgacgattcagacgaggatgagccttgtgccatcagtggcaaatggactttccaaagggacagcaagaggtggtcccggcttgaagagtttgatgtcttttctccaaaacaagacctggtccctgggtccccagacgactcccacccgaaggacggccccagccccggaggcacgctgatggacctcagcgagcgccaggaggtgtcttccgtccgcagcctcagcagcactggcagcctccccagccacgcgccccccagcgaggatgctgccaccccccggactaactccgtcatcagcgtttgctcctccagcaacttggcaggcaatgacgactctttcggcagcctgccctctcccaaggaactgtccagcttcagcttcagcatgaaaggccacgaaaaaactgccaagtccaagacgcgcagtctgctgaaacggatggagagcctgaagctcaagagctcccatcacagcaagcacaaagcgccctcaaagctggggttgatcatcagcgggcccatcttgcaagaggggatggatgaggagaagctgaagcagctcaactgcgtggagatctccgccctcaatggcaaccgcatcaacgtccccatggtacgaaagaggagcgtttccaactccacgcagaccagcagcagcagcagccagtcggagaccagcagcgcggtcagcacgcccagccctgttacgaggacccggagcctcagtgcgtgcaacaagcgggtgggcatgtacttagagggcttcgatcctttcaatcagtcaacatttaacaacgtgatggagcagaactttaagaaccgcgagagctacccagaggacacggtgttctacatccctgaagatcacaagcctggcactttccccaaagctctcaccaatggcagtttctccccctcggggaataacggctctgtgaactggaggacgggaagcttccacggccctggccacatcagcctcaggagggaaaacagtagcgacagccccaaggaactgaagagacgcaattcttccagctccatgagcagccgcctgagcatctacgacaacgtgccgggctccatcctctactccagttcaggggacctggcggatctggagaacgaggacatcttccccgagctggacgacatcctctaccacgtgaaggggatgcagcggatagtcaatcagtggtcggagaagttttctgatgagggagattcggactcagccctggactcggtctctccctgcccgtcctctccaaaacagatacacctggatgtggacaacgaccgaaccacacccagcgacctggacagcacaggcaactccctgaatgaaccggaagagccctccgagatcccggaaagaagggattctggggttggggcttccctaaccaggtccaacaggcaccgactgagatggcacagtttccagagctcacatcggccaagcctcaactctgtatcactacagattaactgccagtctgtggcccagatgaacctgctgcagaaatactcactcctaaagctaacggccctgctggagaaatacacaccttctaacaagcatggttttagctgggccgtgcccaagttcatgaagaggatcaaggttccagactacaaggaccggagtgtgtttggggtcccactgacggtcaacgtgcagcgcacaggacaaccgttgcctcagagcatccagcaggccatgcgatacctccggaaccattgtttggatcaggttgggctcttcagaaaatcgggggtcaagtcccggattcaggctctgcgccagatgaatgaaggtgccatagactgtgtcaactacgaaggacagtctgcttatgacgtggcagacatgctgaagcagtattttcgagatcttcctgagccactaatgacgaacaaactctcggaaacctttctacagatctaccaatatgtgcccaaggaccagcgcctgcaggccatcaaggctgccatcatgctgctgcctgacgagaaccgggaggttctgcagaccctgctttatttcctgagcgatgtcacagcagccgtaaaagaaaaccagatgaccccaaccaacctggccgtgtgcttagcgccttccctcttccatctcaacaccctgaagagagagaattcctctcccagggtaatgcaaagaaaacaaagtttgggcaaaccagatcagaaagatttgaatgaaaacctagctgccactcaagggctggcccatatgatcgccgagtgcaagaagcttttccaggttcccgaggaaatgagccgatgtcgtaattcctataccgaacaagagctgaagcccctcactctggaagcactcgggcacctgggtaatgatgactcagctgactaccaacacttcctccaggactgtgtggatggcctgtttaaagaagtcaaagagaagtttaaaggctgggtcagctactccacttcggagcaggctgagctgtcctataagaaggtgagcgaaggaccccctctgaggctttggaggtcagtcattgaagtccctgctgtgccagaggaaatcttaaagcgcctacttaaagaacagcacctctgggatgtagacctgttggattcaaaagtgatcgaaattctggacagccaaactgaaatttaccagtatgtccaaaacagtatggcacctcatcctgctcgagactacgttgttttaagaacctggaggactaatttacccaaaggagcctgtgcccttttactaacctctgtggatcacgatcgcgcacctgtggtgggtgtgagggttaatgtgctcttgtccaggtatttgattgaaccctgtgggccaggaaaatccaaactcacctacatgtgcagagttgacttaaggggccacatgccagaatggtacacaaaatcttttggacatttgtgtgcagctgaagttgtaaagatccgggattccttcagtaaccagaacactgaaaccaaagacaccaaatctaggtgatcactgaagcaacgcaaccgcttccaccaccatggtgtttgtttctagaacttttgccagtccttgaagaatgggttctgtgtctaatcctgaaacaaagaaaactacaagctggagtgtaggaattgactatagcaatttgatacatttttaaagctgcttcctgtttgttgagggtctgtattcatagaccttgactggaatatgtaagactgtg

SEQ ID NO: 5 - DNA, 915 bp, Artificial Sequence
  Description of Artificial Sequence Synthetic polynucleotide
gttcgttgcaacaaattgatgagcaatgcttttttataatgccaactttgtacaaaaaagttggcatggcaagtccagactggggatatgatgacaaaaatggtcctgaacaatggagcaagctgtatcccattgccaatggaaataaccagtcccctgttgatattaaaaccagtgaaaccaaacatgacacctctctgaaacctattagtgtctcctacaacccagccacagccaaagaaattatcaatgtggggcattccttccatgtaaattttgaggacaacgataaccgatcagtgctgaaaggtggtcctttctctgacagctacaggctctttcagttccattttcactggggcagtacaaatgagcatggttcagaacatacagtggatggagtcaaatattctgccgagcttcacgtagctcactggaattctgcaaagtactccagccttgctgaagctgcctcaaaggctgatggtttggcagttattggtgttttgatgaaggttggtgaggccaacccaaagctgcagaaagtacttgatgccctccaagcaattaaaaccaagggcaaacgagccccattcacaaattttgacccctctactctccttccttcatccctggatttctggacctaccctggctctctgactcatcctcctctttatgagagtgtaacttggatcatctgtaaggagagcatcagtgtcagctcagagcagctggcacaattccgcagccttctatcaaatgttgaaggtgataacgctgtccccatgcagcacaacaaccacccaacccaacctctgaagggcagaacagtgagagcttcattttgcccaactttcttgtacaaagttggcattataagaaagcattgcttatcaatttgttgcaacgaac

SEQ ID NO: 6 - DNA, 628 bp, Homo sapiens
acatttgcttctgacacaactgtgttcactagcaacctcaaacagacaccatggtgcatctgactcctgaggagaagtctgccgttactgccctgtggggcaaggtgaacgtggatgaagttggtggtgaggccctgggcaggctgctggtggtctacccttggacccagaggttctttgagtcctttggggatctgtccactcctgatgctgttatgggcaaccctaaggtgaaggctcatggcaagaaagtgctcggtgcctttagtgatggcctggctcacctggacaacctcaagggcacctttgccacactgagtgagctgcactgtgacaagctgcacgtggatcctgagaacttcaggctcctgggcaacgtgctggtctgtgtgctggcccatcactttggcaaagaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 7 - DNA, 366 bp, Homo sapiens
atgaagcttctcacgggcctggttttctgctccttggtcctgggtgtcagcagccgaagcttcttttcgttccttggcgaggcttttgatggggctcgggacatgtggagagcctactctgacatgagagaagccaattacatcggctcagacaaatacttccatgctcgggggaactatgatgctgccaaaaggggacctgggggtgcctgggctgcagaagtgatcagcgatgccagagagaatatccagagattctttggccatggtgcggaggactcgctggctgatcaggctgccaatgaatggggcaggagtggcaaagaccccaatcacttccgacctgctggcctgcctgagaaatac

SEQ ID NO: 8 - DNA, 981 bp, Homo sapiens
gcaagcttcaagggcccatcggtcttccccctggtgccctgctccaggagcacctccgagagcacagccgccctgggctgcctggtcaaggactacttccccgaaccggtgacggtgtcgtggaactcatgcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccatcatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagggtcctcaccgtcctgcaccaggactggctgaacggtaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggaggacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctccgggtaaa

SEQ ID NO: 9 - DNA, 2078 bp, Homo sapiens
gcaacgcaaagcgcttggtattgagtctgtggccgacttcggttccggtctctgcagcagccgtgatcgcttagtggagtgcttagggtagttggccaggatgccgaatatcaaaatcttcagcggcagctcccaccaggacttatctcagaaaattgctgaccgcctgggcctggagctaggcaaggtggtgactaagaaattcagcaaccaggagacctgtgtggaaattggtgaaagtgtacgtggagaggatgtctacattgttcagagtggttgtggcgaaatcaatgacaatttaatggagcttttgatcatgattaatgcctgcaagattgcttcagccagccgggttactgcagtcatcccatgcttcccttatgcccggcaggataagaaagataagagccgggcgccaatctcagccaagcttgttgcaaatatgctatctgtagcaggtgcagatcatattatcaccatggacctacatgcttctcaaattcagggcttttttgatatcccagtagacaatttgtatgcagagccggctgtcctaaagtggataagggagaatatctctgagtggaggaactgcactattgtctcacctgatgctggtggagctaagagagtgacctccattgcagacaggctgaatgtggactttgccttgattcacaaagaacggaagaaggccaatgaagtggaccgcatggtgcttgtgggagatgtgaaggatcgggtggccatccttgtggatgacatggctgacacttgtggcacaatctgccatgcagctgacaaacttctctcagctggcgccaccagagtttatgccatcttgactcatggaatcttctccggtcctgctatttctcgcatcaacaacgcatgctttgaggcagtagtagtcaccaataccatacctcaggaggacaagatgaagcattgctccaaaatacaggtgattgacatctctatgatccttgcagaagccatcaggagaactcacaatggagaatccgtttcttacctattcagccatgtccctttataatagagtaacttctgaggctttttgagaataaaatccaccccacccttgtttccccttggtatttgatgacaaattcagcagaagacccggcttgctccagtgtagctttctacatcccacatcaggtatattagagcttatccgaactggggaaagacggattgagattaactgctgggacctcctacctgcattatctcattctggcttccttgataattctgtgggccttgcagctttaactatagctcagctgctgcaagatttcagacttttgaggatgttgtgtgagggtgtttgactgtgactggggaagctcagactactttgtatgtgaatgcttcagggttttctttgttgagaacaactagcaacaaaggcaacccatgtgtgaccagttctccccaaggtctatgctaaattatagcaagagccctgggcaaccccaaacctagtcctggtagctgagcaccctgtaaggcaggagcaggcagctcagcttgagcagacattgggtggggggtggggggtggttgaggggggaggcagcacagtgcagcaaatgtttcttgggaggaagaagcctgatccatcaccatctgcttgactatgtagcttggattctcctttgtacctatccctttcgatttggctttaccttcatctatcttgatcctttcctggccaaatatcctcttgggcccaaatgaacattgtaccatagtcttctggaaagcaaacatgcttcctgctatgtaattgctaacattcatattagatgatgtgctgtagcttgatcttccttagcctactgccactgaggcagtaggttttaggtggtatcgtagtgccttttgattaatttaagtatttaattttcatcttccttctttggatctatttggcctctcaaatgaactgagattcctgttaaaaaagattgatgttattgtctcttgtagaggaaactaataaagtgtgtgtacctgtgtgaaaaaaaaaaaaaaaaaaaaaaaaaaaa

SEQ ID NO: 10 - DNA, 1305 bp, Artificial Sequence
  Description of Artificial Sequence Synthetic polynucleotide
gttcgttgcaacaaattgatgagcaatgcttttttataatgccaactttgtacaaaaaagttggcatggttgaagcagatcgcccaggaaagctcttcattggtgggcttaatacggaaacaaatgagaaagctcttgaagcagtatttggcaaatatggacgaatagtggaagtactcttgatgaaagaccgtgaaaccaacaaatcaagaggatttgcttttgtcacctttgaaagcccagcagacgctaaggatgcagccagagacatgaatggaaagtcattagatggaaaagccatcaaggtggaacaagccaccaaaccatcatttgaaagtggtagacgtggaccgcctccacctccaagaagtagaggccctccaagaggtcttagaggtggaagaggaggaagtggaggaaccaggggacctccctcacggggaggacacatggatgacggtggatattccatgaattttaacatgagttcttccaggggaccactcccagtaaaaagaggaccaccaccaagaagtgggggtcctcctcctaagagatctgcaccttcaggaccagttcgcagtagcagtggaatgggaggaagagctcctgtatcacgtggaagagatagttatggaggtccacctcgaagggaaccgctgccctctcgtagagatgtttatttgtccccaagagatgatgggtattctactaaagacagctattcaagcagagattacccaagttctcgtgatactagagattatgcaccaccaccacgagattatacttaccgtgattatggtcattccagttcacgtgatgactatccatcaagaggatatagcgatagagatggatatggtcgtgatcgtgactattcagatcatccaagtggaggttcctacagagattcatatgagagttatggtaactcacgtagtgctccacctacacgagggcccccgccatcttatggtggaagcagtcgctatgatgattacagcagctcacgtgacggatatggtggaagtcgagacagttactcaagcagccgaagtgatctctactcaagtggtcgtgatcgggttggcagacaagaaagagggcttcccccttctatggaaagggggtaccctcctccacgtgattcctacagcagttcaagccgcggagcaccaagaggtggtggccgtggaggaagccgatctgatagagggggaggcagaagcagatactacccaactttcttgtacaaagttggcattataagaaagcattgcttatcaatttgttgcaacgaac

SEQ ID NO: 11 - DNA, 3960 bp, Homo sapiens
gcgggggcgggctttgccgagcgcagagctgcagccgccgagccggacgtgtgcgcgaagatggcgggccggagcatgcaagcggcaagatgtcctacagatgaattatctttaaccaattgttcagttgtgaatgaaaaggatttccagtctggccagcatgtgattgtgaggacctctcccaatcacaggtacacatttacactgaagacacatccatcggtggttccagggagcattgcattcagtttacctcagagaaaatgggctgggctttctattgggcaagaaatagaagtctccttatatacatttgacaaagccaaacagtgtattggcacaatgaccatcgagattgatttcctgcagaaaaaaagcaatgactccaacccttatgacaccgacaagatggcagcagaatttattcagcaattcaacaaccaggcctactcagtgggacaacagcttgtctttagcttcaatgaaaagctttttggcttactggtgaaggacattgaatccatggatcctagcatcctgaagggagagcctgcgacagggaaaaggcagaagattgaagtaggactggttgttggaaacagtcaagttgcatttgaaaaagcagaaaattcgtcacttaatcttattggcaaagctaaaaccaaggaaaatcgccaatcaattatcaatcctgactggaactttgaaaaaatgggaataggaggtctagacaaggaattttcagatattttccgacgagcatttgccttccgagtatttcctccagagattgtggagcagatgggttgtatacatgttaaaggcatcctgttatatggacccccaggttgtggtaagactctcttggctcgacagattggcaagatgttgaatgcaagagagcccaaagtggtcaatgggccagaaatccttaacaaatatgtgggagaatcagaggctaacattcgcaaactttttgctgatgctgaagaggagcaaaggaggcttggtgctaacagtggtttgcacatcatcatctttgatgaaattgatgccatctgcaagcagagagggagcatggctggtagcacgggagttcatgacactgttgtcaaccagttgctgtccaaaattgatggcgtggagcagctaaacaacatcctagtcattggaatgaccaatagaccagatctgatagatgaggctcttcttagacctggaagactggaagttaaaatggagataggcttgccagatgagaaaggccgactacagattcttcacatccacacagcaagaatgagagggcatcagttactctctgctgatgtagacattaaagaactggccgtggagaccaagaatttcagtggtgctgaattggagggtctggtgcgagcagcccagtccactgctatgaatagacacataaaggccagtactaaagtggaagtggacatggagaaagcagaaagcctgcaagtgacgagaggagacttccttgcttctttggagaatgatatcaaaccagcctttggcacaaaccaagaagattatgcaagttacattatgaacggtatcatcaaatggggtgacccagttactcgagttctagatgatggggagctgctggtgcagcagactaagaacagtgaccgcacaccattggtcagcgtgcttctggaaggccctcctcacagtgggaagactgctttagctgcaaaaattgcagaggaatccaacttcccgttcatcaagatctgttctcctgataaaatgattggcttttctgaaacagccaaatgtcaggccatgaagaagatctttgatgatgcgtacaaatcccagctcagttgtgtggttgtggatgacattgagagattgcttgattacgtccctattggccctcgattttcaaatcttgtattacaggctcttctcgttttactgaaaaaggcacctcctcagggccgcaagcttcttatcattgggaccactagccgcaaagatgtccttcaggagatggaaatgcttaacgctttcagcaccaccatccacgtgcccaacattgccacaggagagcagctgttggaagctttggagcttttgggcaacttaaaggataaggaacgcaccacaattgcacagcaagtcaaagggaagaaggtctggataggaatcaagaagttactaatgctgatcgagatgtccctacagatggatcctgaataccgtgtgagaaaattcttggccctcttaagagaagaaggagctagcccccttgattttgattgaaaatgaactatttgaaacacacagtgaccaagggaagtgaccaaggtgaagatggcctaggatcttcactgtcttactcaagatactggactaagtggaacgttctctaccttcaacatgtgctcgctctgcatgattagtgcaataaaactcccttccttatgcatactgagatagcttagtgtctcgtggaaggtgtcaatttggtttagaatgctgcgcttaccttcccatgcaggctaaagtgattccttcttgctcagtccctctgggtgggaaccatccagtacttgtggacactacacgtttcaacctctctactagcaccatcacccttgaaaactctcagtcagtgtcatgaatgttgcatgacaacagttggccgattagaaggcagactttctacatgcaaatctggcttagtaaatcgaggtgtgggccagagatcctctgacagctgtcctgagctaacactaaaagtcactgggtatttggttaaaggtctcccacaagactggtattctctttgcctgaagaaacaaggcattgaatctctaaaatgctgttctcaatcattgtcagagatgttttcaagttgcagtcagaagatctttcttaatagaaagtcagatgactaccgtgttggttgtgacttccccttaagtataactaatttgctctgtggtaagagatatgctcattattaccacttagaagatgttgttaaaaacatgtgaaagataggtatggaaaaagcatacacccccaaacagaaaggagttattaaagtaatttacaaacctctcagcactaattagtgtccaactccaagtgggtcaattccttagtataatattaaggcttactagtatcactgctttttccttagcttaatgacttacttagaatttatcctttattttaaatgatctgtactatctagtgtctaaaacactattctccagaaaaatcaatcattttctagccctctccctcagtcctttattgtccattccaatacattgaacacatttcctttaccctccacacacttcttccaaaaggaagcacccgttgagtccttttgagggtgatttgtcttacaactgactgacttagcaggaatttaattaggtcatatttggtgatgagacttatggagtgtgcctctctctcccaactgctgcttaaaatgcaaggacaagcaattagaagccatcctaaggtgcttacctcacacgccacccatgaggcttgtggccacagtggcacttgggtgtggctcctctgttatttgtcctcatgtgagaaagcagatcatctccaaatcttgccatttgtatacttttggtggagacttggatgtcatatcttctttgttttgggttttcttccctagcttattttgtggcttttaaagaagtggattgtattgtgagatcctgtgattcctggtggccagtatcctggattcctctaagatcttgcctctttcctcctcatgaaagcagcacacattgtgttaacttatgtctcttgttaaatgagcttaatgtctttgtgttttgtccaaaactgtattgaaaaaatattgtttaatgcaaatgaaggaatgcaataaagagtaaatatacttgaaaaaaaaaaaaaaaaaaaaaaaaaaaa

SEQ ID NO: 12 - DNA, 328 bp, Homo sapiens
gtgatgacccagactccactctctctgtccgtcacccctggacagccggcctccatctcctgcaagtctagtcagagcctcctgcatagtgatggaaagacctatttgtattggtacctgcagaagccaggccagtctccacagctcctgatctatgaagtttccaaccggttctctggagtgccagataggttcagtggcagcgggtcagggacagatttcacactgaaaatcagccgggtggaggctgaggatgttggggtttattactgcatgcaaagtatacagccttacacttttggccaggggaccaaggtggagatcaaac

SEQ ID NO: 13 - DNA, 1866 bp, Homo sapiens
gtggcgagttccggatccctgcctagcgcggcccaacctttactccagagatcatggctgccgaggatgtggtggcgactggcgccgacccaagcgatctggagagcggcgggctgctgcatgagattttcacgtcgccgctcaacctgctgctgcttggcctctgcatcttcctgctctacaagatcgtgcgcggggaccagccggcggccagcggcgacagcgacgacgacgagccgccccctctgccccgcctcaagcggcgcgacttcacccccgccgagctgcggcgcttcgacggcgtccaggacccgcgcatactcatggccatcaacggcaaggtgttcgatgtgaccaaaggccgcaaattctacgggcccgaggggccgtatggggtctttgctggaagagatgcatccaggggccttgccacattttgcctggataaggaagcactgaaggatgagtacgatgacctttctgacctcactgctgcccagcaggagactctgagtgactgggagtctcagttcactttcaagtatcatcacgtgggcaaactgctgaaggagggggaggagcccactgtgtactcagatgaggaagaaccaaaagatgagagtgcccggaaaaatgattaaagcattcagtggaagtatatctatttttgtattttgcaaaatcatttgtaacagtccactctgtctttaaaacatagtgattacaatatttagaaagttttgagcacttgctataagttttttaattaacatcactagtgacactaataaaattaacttcttagaatgcatgatgtgtttgtgtgtcacaaatccagaaagtgaactgcagtgctgtaatacacatgttaatactgtttttcttctatctgtagttagtacaggatgaatttaaatgtgtttttcctgagagacaaggaagacttgggtatttcccaaaacaggtaaaaatcttaaatgtgcaccaagagcaaaggatcaacttttagtcatgatgttctgtaaagacaacaaatccctttttttttctcaattgacttaactgcatgatttctgttttatctacctctaaagcaaatctgcagtgttccaaagactttggtatggattaagcgctgtccagtaacaaaatgaaatctcaaaacagagctcagctgcaaaaaagcatattttctgtgtttctggactgcactgttgtccttgccctcacatagacactcagacaccctcacaaacacagtagtctatagttaggattaaaataggatctgaacattcaaaagaaagctttggaaaaaaagagctggctggcctaaaaacctaaatatatgatgaagattgtaggactgtcttcccaagccccatgttcatggtggggcaatggttatttggttattttactcaattggttactctcatttgaaatgagggagggacatacagaataggaacaggtgtttgctctcctaagagccttcatgcacacccctgaaccacgaggaaacagtacagtcgctagtcaagtggtttttaaagtaaagtatattcataaggtaacagttattctgttgttataaaactatacccactgcaaaagtagtagtcaagtgtctaggtctttgatattgctcttttggttaacactaagcttaagtagactatacagttgtatgaatttgtaaaagtatatgaacacctagtgagatttcaaacttgtaattgtggttaaatagtcattgtattttcttgtgaactgtgttttatgattttacctcaaatcagaaaacaaaatgatgtgctttggtcagttaataaaaatggttttacccacaaaaaaaaaaaaaaa

(19) United States
(12) Patent Application Publication
(10) Pub. No.: US 2021/0046088 A1
(43) Pub. Date: Feb. 18, 2021

(54) METHODS AND COMPOSITIONS FOR THE DIAGNOSIS AND TREATMENT OF ENDOMETRIOSIS AND ENDOMETRIOSIS-RELATED DISORDERS

(71) Applicant: Board of Trustees of Michigan State University, East Lansing, MI (US)

(72) Inventors: Asgerally T. Fazleabas, Ada, MI (US); Irving Vega, Ada, MI (US); Genna Wilbur, Lake Orion, MI (US); Andrew Umstead, Grand Rapids, MI (US)

(21) Appl. No.: 16/929,403

(22) Filed: Jul. 15, 2020

Related U.S. Application Data
(60) Provisional application No. 62/874,544, filed on Jul. 16, 2019.

(51) Int. Cl. A61K 31/57; A61P 15/00; G01N 33/574

(52) U.S. Cl. CPC A61K 31/57; G01N 33/57449; G01N 33/57442; A61P 15/00

(57) ABSTRACT

Provided herein are methods of determining whether a subject is afflicted with an endometriosis-related condition, such as endometriosis. Also provided herein are therapies for endometriosis-related conditions.

### RELATED APPLICATIONS

This application claims priority to U.S. Provisional Application No. 62/874,544, filed Jul. 16, 2019, which is incorporated herein by reference in its entirety.

### STATEMENT OF RIGHTS

This invention was made with government support under HD083273 awarded by the National Institutes of Health. The government has certain rights in the invention.

### BACKGROUND OF THE INVENTION

Endometriosis is a gynecological disease characterized by the presence and growth of endometrial tissue outside of the uterus and affects approximately ten to fifteen percent of reproductive aged women. The standard diagnostic method for endometriosis is surgical pelvic laparoscopy where the presence of disease is confirmed by direct visualization. This invasive diagnostic method combined with nonspecific symptoms such as pelvic pain, dysmenorrhea, dyspareunia, and infertility, has resulted in an average eight-year gap between the onset of disease to time of diagnosis. An accurate and less invasive diagnostic method is a significant unmet need for women's health.

### SUMMARY OF THE INVENTION

Described herein are diagnostic methods, assays, and systems, as well as methods of treatment, for endometriosis and related conditions (e.g., endometriosis cysts and ovarian cancer). The invention described herein is based, in part, that tissue levels of products of KRT1, PZP, KRT14, HP, CA1, HBB, SAA1, IGHG4, PRPS1, RBMX, NSF, IGKV2D-29, and/or PGRMC1 are elevated in subjects with endometriosis.

Also provided herein are treatments of, e.g. endometriosis-related conditions with modulators of protein and RNA products of KRT1, PZP, KRT14, HP, CA1, HBB, SAA1, IGHG4, PRPS1, RBMX, NSF, IGKV2D-29, and/or PGRMC1, including, for example, inhibitors or binding factors of products of KRT1, PZP, KRT14, HP, CA1, HBB, SAA1, IGHG4, PRPS1, RBMX, NSF, IGKV2D-29, and/or PGRMC1.

Provided herein are methods of determining whether a subject has an endometriosis-related condition. In some embodiments, the methods comprise measuring the levels of or activity of at least one protein (e.g., at least two, at least three, at least four, at least five, at least six, at least seven, at least eight, at least nine, at least ten, at least eleven, at least twelve, at least thirteen, at least fourteen, or at least fifteen proteins) encoded by at least one gene (e.g., at least two, at least three, at least four, at least five, at least six, at least seven, at least eight, at least nine, at least ten, at least eleven, at least twelve, or all thirteen) selected from KRT1, PZP, KRT14, HP, CA1, HBB, SAA1, IGHG4, PRPS1, RBMX, NSF, IGKV2D-29, and PGRMC1 in a biological sample isolated from the subject, and if the level of or activity of the protein(s) is elevated compared to a level or activity of the protein(s) in a biological sample from a subject who is not afflicted with an endometrioses-related condition, the subject is determined to have the endometrioses-related condition. For example, the methods provided herein may comprise measuring the levels of or activity of at least two proteins, wherein each protein is encoded by a different gene (e.g., different genes disclosed herein).

The levels or activity of the at least one protein(s) may be measured by any technique known in the art, including but not limited to contacting the biological sample with an antibody specific for the at least one protein(s).

The methods described herein also comprise administering to the subject a therapy for the endometriosis-related condition if the subject is determined to have an endometriosis-related condition.

In some aspects, provided herein are methods of determining whether a subject has endometriosis-related condition by measuring the levels of an at least one RNA product (e.g., at least two, at least three, at least four, at least five, at least six, at least seven, at least eight, at least nine, at least ten, at least eleven, at least twelve, at least thirteen, at least fourteen, or at least fifteen RNA products) of at least one gene (e.g., at least two, at least three, at least four, at least five, at least six, at least seven, at least eight, at least nine, at least ten, at least eleven, at least twelve, or all thirteen) selected from KRT1, PZP, KRT14, HP, CA1, HBB, SAA), IGHG4, PRPS1, RBMX, NSF, IGKV2D-29, and PGRMC1 in a biological sample isolated from the subject. In some embodiments, if the level of the at least one RNA product is elevated compared to a level of the RNA product in a subject who is not afflicted with an endometrioses-related condition, the subject is determined to have an endometrioses-related condition.

Also provided herein are methods of treating or preventing an endometriosis-related condition in a subject in need thereof by measuring the levels of or activity of at least one protein encoded by at least one gene selected from KRT1, PZP, KRT14, HP, CA1, HBB, SAA1, IGHG4, PRPS1, RBMX, NSF, IGKV2D-29, and PGRMC1 in a biological sample isolated from the subject, and if the level of or activity of the protein(s) is elevated compared to a level or activity of the protein(s) in a biological sample from a subject who is not afflicted with an endometrioses-related condition, administering to the subject a therapy for the endometriosis-related condition.

In some aspects, provided herein are methods of treating or preventing an endometriosis-related condition in a subject in need thereof by measuring the levels of an at least one RNA product of at least one gene selected from KRT1, PZP, KRT14, HP, CA1, HBB, SAA1, IGHG4, PRPS1, RBMX, NSF, IGKV2D-29, and PGRMC1 in a biological sample isolated from the subject, and if the level of the at least one RNA product(s) is elevated compared to a level of the RNA product(s) in a subject who is not afflicted with an endometrioses-related condition, administering to the subject a therapy for the endometriosis-related condition. The levels of RNA product, protein activity, or protein levels may be significantly higher than an individual not afflicted with an endometrioses-related condition.

The therapy may be any therapy administered to a subject afflicted with an endometriosis-related condition (e.g., hormonal therapy). The biological sample may be endometrial tissue, cervical tissue, cervical cells, or uterine tissue. The biological sample may be obtained by non-invasive methods, e.g., a cervical swab. The endometriosis-related condition may be, for example, endometriosis, endometriosis cysts, endometrioid cancer, or ovarian cancer.

### BRIEF DESCRIPTION OF THE DRAWINGS

FIG. 1 shows study design to develop a profile of differentially expressed proteins (DEPs) throughout the course of disease.

FIG. 2 is a method summary of the investigation to discern differentially expressed proteins throughout the course of disease.

FIG. 3 shows the results of the investigation to discern differentially expressed proteins throughout the course of disease.

FIG. 4 shows independent analysis of human uterine lavage samples from women with and without disease.

FIG. 5 shows the differential expression of biomarkers in humans and baboons.

### DETAILED DESCRIPTION OF THE INVENTION

Provided herein are methods of determining whether a subject has an endometriosis-related condition. In some embodiments, the methods comprise measuring the levels of or activity of at least one protein (e.g., at least two, at least three, at least four, at least five, at least six, at least seven, at least eight, at least nine, at least ten, at least eleven, at least twelve, at least thirteen, at least fourteen, or at least fifteen) encoded by at least one gene (e.g., at least two, at least three, at least four, at least five, at least six, at least seven, at least eight, at least nine, at least ten, at least eleven, at least twelve, or all thirteen) selected from KRT1, PZP, KRT14, HP, CA1, HBB, SAA1, IGHG4, PRPS1, RBMX, NSF, IGKV2D-29, and PGRMC1 in a biological sample isolated from the subject, and if the level of or activity of the protein(s) is elevated compared to a level or activity of the protein(s) in a biological sample from a subject who is not afflicted with an endometrioses-related condition, the subject is determined to have the endometrioses-related condition.

In some aspects, provided herein are methods of determining whether a subject has endometriosis-related condition by measuring the levels of an at least one RNA product (e.g., at least two, at least three, at least four, at least five, at least six, at least seven, at least eight, at least nine, at least ten, at least eleven, at least twelve, at least thirteen, at least fourteen, or at least fifteen) of at least one gene (e.g., at least two, at least three, at least four, at least five, at least six, at least seven, at least eight, at least nine, at least ten, at least eleven, at least twelve, or all thirteen) selected from KRT1, PZP, KRT14, HP, CA1, HBB, SAA1, IGHG4, PRPS1, RBMX, NSF, IGKV2D-29, and PGRMC1 in the subject. In some embodiments, if the level of the at least one RNA product is elevated compared to a level of the RNA product in a subject who is not afflicted with an endometrioses-related condition, the subject is determined to have an endometrioses-related condition.

### I. Definitions

The articles “a” and “an” are used herein to refer to one or to more than one (i.e. to at least one) of the grammatical object of the article. By way of example, “an element” means one element or more than one element.

The term “altered amount” or “altered level” refers to increased or decreased level of a biomarker nucleic acid, e.g., increased or decreased expression level in a sample from a subject with endometriosis or endometriosis-associated disorder, as compared to the expression level of the biomarker nucleic acid in a control sample (e.g., a sample from a person not diagnosed with endometriosis, or a previous sample taken from the subject). The term “altered amount” of a biomarker also includes an increased or decreased protein level of a biomarker protein in a sample, e.g., an endometrioses sample, as compared to the corresponding protein level in a normal or control sample.

The amount of a biomarker in a subject is “significantly” higher or elevated compared to the normal amount of the biomarker, if the amount of the biomarker is greater, respectively, than the normal level by an amount greater than the standard error of the assay employed to assess amount, and preferably at least 1%, 5%, 10%, 20%, 30%, 40%, 50%, 60%, 70%, 80%, 90%, 100%, 150%, 200%, 300%, 350%, 400%, 500%, 600%, 700%, 800%, 900%, 1000% or than that amount. Alternately, the amount of the biomarker in the subject can be considered “significantly” higher or elevated when compared to the normal amount if the amount is at least about two, and or at least about three, four, or five times, higher or lower, respectively, than the normal amount of the biomarker. Such “significance” can also be applied to any other measured parameter described herein, such as for expression, inhibition, cytotoxicity, cell growth, and the like.

As used herein, the term “administering” means providing an agent or composition to a subject, and includes, but is not limited to, administering by a medical professional and self-administering.

As used herein, an “effective amount” is an amount effective in treating or preventing a disease, including, for example, endometriosis.

As used herein, “endometriosis” refers to a condition characterized by the growth of endometrial cells (i.e. cells usually found in the lining of the uterus) outside of the uterine cavity, e.g. on the peritoneum. Endometriosis can occur on any tissue or organ, including, but not limited to, the peritoneum, the rectum, the ovary, and the fallopian tube. Endometriosis is generally a non-malignant condition. Non-limiting signs and symptoms of endometriosis can include pelvic pain, infertility, constipation, chronic fatigue, dysmenorrhea, dyspareunia, dysuria, leg pain, rectal pain, inflammation, and swelling. Early-stage endometriosis can appear as flat patches or flecks on the affected tissue. In some cases, endometriosis progresses to form endometriosis cysts, which can be filled with blood. Endometriosis can also lead to adhesions. As used herein, “endometriosis-related condition” refers to a group of conditions and/or diseases including endometriosis and conditions that are caused by and/or arise from the effects and/or progression of endometriosis (see, e.g. Sayasneh et al. Obstetrics and Gynecology 2011 2011:140310; which is incorporated by reference herein in its entirety). Examples of endometriosis-related conditions include, but are not limited to, endometriosis; endometriosis cysts; endometrioid cancer; ovarian cancer; clear cell cancer; and ovarian clear cell cancer. In some embodiments, an endometriosis-related condition can be endometriosis, endometriosis cysts, endometrioid ovarian cancer, and/or ovarian clear cell cancer. In some embodiments, an endometriosis-related condition can be endometriosis, endometriosis cysts, and/or ovarian clear cell cancer. In some embodiments, an endometriosis-related condition can be a non-cancerous condition, i.e. endometriosis and/or endometriosis cysts.

Endometrial polyps and/or endometriosis itself can lead to and/or involve fibrosis of the affected tissues. Endometrial polyps in particular are very fibrous, and uterine fibroids are a significant health concern. In some embodiments, the methods, assays, and systems described herein can relate to treating a subject with fibrosis, e.g. endometrial fibrosis and/or ovarian fibrosis. These types of fibrosis can contribute to infertility. Accordingly, the methods, assays, and systems described herein can relate to treating a subject with infertility and/or treating a subject in need of treatment to increase fertility. In some embodiments, the subject can be human.

As used herein, the phrase “pharmaceutically-acceptable carrier” means a pharmaceutically-acceptable material, composition or vehicle, such as a liquid or solid filler, diluent, excipient, or solvent encapsulating material, involved in carrying or transporting an agent from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; and (22) other non-toxic compatible substances employed in pharmaceutical formulations.

As used herein, the term “subject” means a human or non-human animal selected for treatment or therapy. In certain embodiments, of the methods and compositions described herein the subject is a human subject.

The phrases “therapeutically-effective amount” and “effective amount” as used herein means the amount of an agent which is effective for producing the desired therapeutic effect in at least a sub-population of cells in a subject at a reasonable benefit/risk ratio applicable to any medical treatment.

“Treating” a disease in a subject or “treating” a subject having a disease refers to subjecting the subject to a pharmaceutical treatment, e.g., the administration of a drug, such that at least one symptom of the disease is decreased or prevented from worsening.

### Biomarkers for Endometriosis Related Conditions:

There is a known and definite correspondence between the amino acid sequence of a particular protein and the nucleotide sequences that can code for the protein, as defined by the genetic code (shown below). Likewise, there is a known and definite correspondence between the nucleotide sequence of a particular nucleic acid and the amino acid sequence encoded by that nucleic acid, as defined by the genetic code.

GENETIC CODE

| | |
|---|---|
| Alanine (Ala, A) | GCA, GCC, GCG, GCT |
| Arginine (Arg, R) | AGA, ACG, CGA, CGC, CGG, |
| | CGT |
| Asparagine (Asn, N) | AAC, AAT |
| Aspartic acid (Asp, D) | GAC, GAT |
| Cysteine (Cys, C) | TGC, TGT |
| Glutamic acid (Glu, E) | GAA, GAG |
| Glutamine (Gln, Q) | CAA, CAG |
| Glycine (Gly, G) | GGA, GGC, GGG, GGT |
| Histidine (His, H) | CAC, CAT |
| Isoleucine (Ile, I) | ATA, ATC, ATT |
| Leucine (Leu, L) | CTA, CTC, CTG, CTT, TTA, |
| | TTG |
| Lysine (Lys, K) | AAA, AAG |
| Methionine (Met, M) | ATG |
| Phenylalanine (Phe, F) | TTC, TTT |
| Proline (Pro, P) | CCA, CCC, CCG, CCT |
| Serine (Ser, S) | AGC, AGT, TCA, TCC, TCG, |
| | TCT |
| Threonine (Thr, T) | ACA, ACC, ACG, ACT |
| Tryptophan (Trp, W) | TGG |
| Tyrosine (Tyr, Y) | TAC, TAT |
| Valine (Val, V) | GTA, GTC, GTG, GTT |
| Termination signal | TAA, TAG, TGA |
| (end) | |

An important and well-known feature of the genetic code is its redundancy, whereby, for most of the amino acids used to make proteins, more than one coding nucleotide triplet may be employed (illustrated above). Therefore, a number of different nucleotide sequences may code for a given amino acid sequence. Such nucleotide sequences are considered functionally equivalent since they result in the production of the same amino acid sequence in all organisms (although certain organisms may translate some sequences more efficiently than they do others). Moreover, occasionally, a methylated variant of a purine or pyrimidine may be found in a given nucleotide sequence. Such methylations do not affect the coding relationship between the trinucleotide codon and the corresponding amino acid.

In view of the foregoing, the nucleotide sequence of a DNA or RNA encoding a biomarker nucleic acid (or any portion thereof) can be used to derive the polypeptide amino acid sequence, using the genetic code to translate the DNA or RNA into an amino acid sequence. Likewise, for polypeptide amino acid sequence, corresponding nucleotide sequences that can encode the polypeptide can be deduced from the genetic code (which, because of its redundancy, will produce multiple nucleic acid sequences for any given amino acid sequence). Thus, description and/or disclosure herein of a nucleotide sequence which encodes a polypeptide should be considered to also include description and/or disclosure of the amino acid sequence encoded by the nucleotide sequence. Similarly, description and/or disclosure of a polypeptide amino acid sequence herein should be considered to also include description and/or disclosure of all possible nucleotide sequences that can encode the amino acid sequence.

Finally, nucleic acid and amino acid sequence information for the loci and biomarkers of the present invention (e.g., biomarkers and associated Accession Numbers listed in Table 1, Table 2, or Table 3) are well-known in the art and readily available on publicly available databases, such as the National Center for Biotechnology Information (NCBI). Table 1 is not an exhaustive list of all transcript or isoform variants that are included in the present invention. The present invention includes, but is not limited to, microRNA, mRNA, or peptide products as biomarkers of any gene disclosed herein (e.g., any microRNA, mRNA, or peptide products of a gene or RNA listed in Tables 1-3).

TABLE 1
SEQ ID NO: 1 Homo sapiens keratin 1 (KRT1), mRNA (NM_006121.4)

| | |
|---|---|
| 1 | agaggagtgt ttagctcctt cccttactct accttgctcc tacttttctc taagtcaaca |
| 61 | tgagtcgaca gtttagttcc aggtctgggt accgaagtgg agggggcttc agctctggct |
| 121 | ctgctgggat catcaactac cagcgcagga ccaccagcag ctccacacgc cgcagtggag |
| 181 | gaggtggtgg gagattttca agctgtggtg gtggtggtgg tagctttggt gctggtggtg |
| 241 | gatttggaag tcggagtctt gttaaccttg gtggcagtaa aagcatctcc ataagtgtgg |
| 301 | ctagaggagg tggacgtggt agtggctttg gtggtggtta tggtggtggt ggctttggtg |
| 361 | gtggtggctt tggtggtggt ggctttggtg gaggtggcat tgggggtggt ggctttggtg |
| 421 | gttttggcag tggtggtggt ggttttggtg gaggtggctt tgggggtggt ggatatgggg |
| 481 | gtggttatgg tcctgtctgc cctcctggtg gcatacaaga agtcactatc aaccagagcc |
| 541 | ttcttcagcc cctcaatgtg gagattgacc ctgagatcca aaaggtgaag tctcgagaaa |
| 601 | gggagcaaat caagtcactc aacaaccaat ttgcctcctt cattgacaag gtgaggttcc |
| 661 | tggagcagca gaaccaggta ctgcaaacaa aatgggagct gctgcagcag gtagatacct |
| 721 | ccactagaac ccataattta gagccctact ttgagtcatt catcaacaat ctccgaagga |
| 781 | gagtggacca actgaagagt gatcaatctc ggttggattc ggaactgaag aacatgcagg |
| 841 | acatggtgga ggattaccgg aacaagtatg aggatgaaat caacaagcgg acaaatgcag |
| 901 | agaatgaatt tgtgaccatc aagaaggatg tggatggtgc ttatatgacc aaggtggacc |
| 961 | ttcaggccaa acttgacaac ttgcagcagg aaattgattt ccttacagca ctctaccaag |
| 1021 | cagagttgtc tcagatgcag actcaaatca gtgaaactaa tgtcatcctc tctatggaca |
| 1081 | acaaccgcag tctcgacctg gacagcatca ttgctgaggt caaggcccag tacgaggata |
| 1141 | tagcccagaa gagcaaagct gaggccgagt ccttgtacca gagcaagtat gaagagctgc |
| 1201 | agatcactgc tggcagacat ggggatagtg tgagaaattc aaagatagaa atttctgagc |
| 1261 | tgaatcgtgt gatccagaga cttagatctg aaatcgacaa tgtcaagaag cagatctcca |
| 1321 | acttgcagca gtccatcagt gatgcagagc agcgtggcga gaatgccctc aaggatgcca |
| 1381 | agaacaagct gaatgacctg gaggatgccc tgcagcaggc caaggaagac ctggcccgcc |
| 1441 | tgctgcgcga ctaccaggag ctgatgaaca caaagctggc cctggatctg gagattgcca |
| 1501 | cctacaggac cctcctggag ggagaagaaa gcaggatgtc tggagaatgt gccccgaacg |
| 1561 | tgagtgtgtc tgtgagcaca agccacacca ccatcagtgg aggtggcagc cgaggaggtg |
| 1621 | gcggcggtgg ctacggctct ggaggtagca gctatggctc cggaggtggt agctatggtt |
| 1681 | ctggaggtgg cggcggcggc ggccgtggca gctatggctc cggaggtagc agctacggct |
| 1741 | ccggaggtgg cagctatggc tctggaggtg gcggcggcgg ccatggcagc tacggctccg |
| 1801 | gaagcagcag tgggggctac agaggtggct ctggaggcgg cggcggcggc agctctggcg |
| 1861 | gccggggctc tggcggcggg agctctggag gctccatagg aggccgggga tccagctctg |
| 1921 | ggggtgtcaa gtcctctggt ggcagttcca gcgtgaagtt tgtttctacc acttattccg |
| 1981 | gagtaaccag ataaagagat gccctctgtt tcattagctc tagttctccc ccagcatcac |
| 2041 | taacaaatat gcttggcaag accgaggtcg atttgtccca gccttaccgg agaaaagagc |
| 2101 | tatggttagt tacactagct catcctattc ccccagctct ttcttttctg ctgtttccca |
| 2161 | atgaagtttt cagatcagtg gcaatctcag tcccctggct atgaccctgc tttgttcttt |
| 2221 | ccctgagaaa cagttcagca gtgaccacca cccacatgac atttcaaagc acctccttaa |
| 2281 | gccagccaga gtaggaccag ttagacccag ggtgtggaca gctccttagc atcttatctc |
| 2341 | tgtgctgttt tggttttgta cataaggtgt aagcaagttg tttttctttt gtggagaggt |
| 2401 | cttaaactcc ccatttcctt gttttgctgc aataaactgc atttgaaatt c |

SEQ ID NO: 2 Homo sapiens PZP alpha-2-macroglobulin like (PZP), mRNA
(NM_002864.3)

| | |
|---|---|
| 1 | agggcgaggc tgagggcaga gagttggaca caaccctgag atttatccct cacaatgcgg |
| 61 | aaagacagac ttcttcattt atgtcttgtg ctacttctta tcctgctttc tgccagtgac |
| 121 | tcaaactcta cagaaccgca gtatatggtg ctggtcccct ccctgctcca cactgaggcc |
| 181 | cctaagaagg gctgtgtcct tctgagccac ctgaatgaga cagtgactgt aagtgcttcc |
| 241 | ttggagtctg gcagggaaaa caggagcctc ttcactgacc tggtggcgga gaaggactta |
| 301 | ttccactgtg tctccttcac tctcccaagg atctcagcct cttcagaggt ggcattcctt |
| 361 | agcatccaga taaaggggcc tacgcaagat ttcaggaaga ggaacacagt tctggtactg |
| 421 | aacacccaaa gtctggtctt tgtccagaca gacaaaccca tgtataaacc aggacagaca |
| 481 | gtaagattcc gtgttgtctc cgtggatgaa aattttcgcc ctcgaaatga actgattcca |
| 541 | ctgatatacc ttgagaaccc aagaagaaat cgaattgcac aatggcagag tctcaagcta |
| 601 | gaagctggca tcaatcagtt gtcctttccc ctctcatcag agcccattca gggctcctac |
| 661 | agggtggtgg tacagacaga atcaggtgga aggatacagc accccttcac cgtggaggaa |
| 721 | tttgtgcttc ccaagtttga ggtcaaagtt caggtgccaa agataatcag tatcatggat |
| 781 | gaaaaagtga acataacagt ctgtggagaa tacacttatg ggaagcctgt cccaggactt |
| 841 | gcaactgtga gcctgtgtag aaaattatct cgtgttctta attgtgacaa gcaggaggtc |
| 901 | tgtgaggaat tcagtcaaca gcttaacagc aatggctgca tcacccaaca agtacacacc |
| 961 | aaaatgctcc agattacaaa tacgggcttt gaaatgaagc ttagagtgga agccaggatc |
| 1021 | agagaagagg ggacagacct ggaagtcact gcaaacagga tcagtgaaat cacaaacatt |
| 1081 | gtatccaaac tcaaattcgt gaaagtggat tcacacttta gacaaggaat cccctttttt |
| 1141 | gcacaggtgc ttctggtgga tggaaaaggt gtgcccatcc ccaataaact cttcttcatc |
| 1201 | tctgtgaatg acgccaatta ttactccaat gcaaccacca atgagcaggg tcttgcacag |
| 1261 | ttttcaatca atactaccag tatctcggtt aataaacttt ttgtccgggt tttcactgtg |
| 1321 | catcccaact tgtgttttca ctattcatgg gtagcagaag accaccaggg tgctcagcac |
| 1381 | actgcaaatc gtgttttctc cttaagtgga agttacattc acctggagcc tgtggctggt |
| 1441 | accctgccct gtggccacac ggagactatc acggcacact atacactgaa tagacaggcc |
| 1501 | atgggagagt tatcggagct cagtttccat tacctgatca tggctaaggg agtcatcgtc |
| 1561 | agatctggaa cccacactct gcctgtggag tcaggagaca tgaaaggcag ttttgcctta |
| 1621 | tccttccctg tggagtcaga cgttgccccc attgcacgaa tgttcatctt tgccatttta |
| 1681 | ccagatggag aagttgttgg agactctgaa aaatttgaga ttgaaaactg tctagccaac |
| 1741 | aaggtggatt tgagcttcag cccagcacaa agtcccccag cctcacatgc ccacctgcaa |
| 1801 | gtagcagctg ctccgcagtc cctctgtgcc cttcgtgctg tggaccaaag tgtgctgctc |
| 1861 | atgaagcctg aggctgagct ctctgtgtcc tcagtatata atctgctaac tgtgaaggat |
| 1921 | ctcaccaatt ttcctgacaa tgtggaccag caggaggaag aacaaggaca ctgtccccgt |
| 1981 | cctttcttca ttcataatgg agccatctat gttcccttat caagtaatga agcagatatt |
| 2041 | tatagcttcc tcaaggggat gggattgaag gtgttcacta actcaaaaat ccgaaaacca |
| 2101 | aagtcgtgtt cagtcatccc ttccgtgtct gcaggagcag taggtcaagg atactatgga |
| 2161 | gcaggtctag gagtagtaga gagaccatat gttcctcaat taggcacata taatgtgata |
| 2221 | cccttaaata atgaacaaag ttcagggcca gtccctgaaa cggtgcgaag ctattttcct |
| 2281 | gagacttgga tctgggagtt ggtggcagtg aactcatcag gtgtggctga ggtaggagta |
| 2341 | acagtccctg acaccatcac cgagtggaag gcaggggcct tctgcctgtc cgaagatgct |
| 2401 | ggacttggta tctcttccac tgcctctctc cgagccttcc agcccttctt tgtggagctc |
| 2461 | acaatgcctt actctgtgat tcgtggagag gtcttcacac tcaaggccac ggtcctaaac |
| 2521 | taccttccca aatgcatccg ggtcagtgtg cagctgaaag cctctccagc cttcctagct |
| 2581 | tcccaaaata caaagggaga agaatcctat tgtatctgtg gaaatgagag acaaaccttg |
| 2641 | tcttggacag tgactcctaa aactctgggg aatgtgaact tctcagtgag tgcagaggca |
| 2701 | atgcagtcct tagaactctg tggaaatgag gttgttgagg tccctgagat taaaagaaaa |
| 2761 | gacacagtca tcaaaaccct gttggtggag gctgaaggta ttgagcaaga aaagactttc |
| 2821 | agttctatga cctgtgcctc aggtgctaat gtgtctgagc agttgtcctt gaagctccca |
| 2881 | tcaaatgtgg tcaaagaatc tgccagagct tctttctcag ttctgggtga catattaggt |
| 2941 | tctgctatgc aaaatataca aaatctcctc cagatgccat atggctgtgg agaacagaac |
| 3001 | atggtcctat ttgctcctaa catctatgtc ttgaactatc tgaatgaaac ccagcagctg |
| 3061 | acgcaggaga tcaaggccaa ggccgttggc tatctcatca ctggttacca gagacagctg |
| 3121 | aactacaaac accaagatgg ctcctacagc acctttgggg aacgatatgg caggaaccag |
| 3181 | ggcaacactt ggctcacagc ttttgtactg aagactttcg cccaggctcg atcctacatc |
| 3241 | ttcattgatg aagcacacat tacccaatct ctcacgtggc tctcccagat gcagaaggac |
| 3301 | aatggctgtt tcaggagctc tgggtcactg ctcaacaatg ccataaaggg aggtgtagaa |
| 3361 | gatgaagcga ccctctccgc ctatgttact attgcccttc tggaaattcc tctcccagtc |
| 3421 | actaacccta ttgttcgcaa tgccctgttc tgcctggagt cagcctggaa tgtagcaaag |
| 3481 | gaggggaccc atgggagcca tgtctacacc aaggcattgc tggcctatgc tttttcccta |
| 3541 | ctgggaaagc aaaatcagaa tagagaaata ctgaactcac ttgataagga agctgtgaaa |
| 3601 | gaagacaacc tcgtccattg ggagcgccct cagagaccca aggcaccagt ggggcatctt |
| 3661 | taccaaaccc aggctccctc tgctgaggtg gagatgacat cctatgtgct cctcgcttat |
| 3721 | ctcacggccc agccagcccc cacctcaggg gacctgacct ctgcaactaa cattgtgaag |
| 3781 | tggatcatga agcagcagaa cgcccaaggt ggtttctcct ccacccagga cacagtggtg |
| 3841 | gctctccatg ccctgtccag gtatggagca gccactttca ccagaactga gaaaactgca |
| 3901 | caggtcaccg ttcaggattc acagaccttt tctacaaatt tccaagtaga caacaacaac |
| 3961 | ctcctattac tgcagcagat ctcattgcca gagctccctg gagaatatgt cataacagta |
| 4021 | actggggaaa gatgtgtgta tcttcagaca tccatgaaat acaatattct tccagagaaa |
| 4081 | gaggactccc catttgcttt aaaagtgcag actgtgcccc aaacttgcga tggacacaaa |
| 4141 | gcccacacca gctttcagat ctcactgacc atcagttaca caggaaaccg tcctgcttcc |
| 4201 | aatatggtga ttgttgatgt aaagatggta tctggtttta ttcccctgaa accaacagta |
| 4261 | aaaatgcttg aaagatctag ctctgtgagc cggacagaag tgagcaacaa ccatgtcctc |
| 4321 | atttatgtgg aacaggtgac aaatcagacg ctaagttttt ccttcatggt tctgcaagac |
| 4381 | atcccagtag gagacttgaa gccagcaatt gttaaagtct atgattacta tgagacagat |
| 4441 | gagtctgtgg ttgctgagta tatcgccccc tgcagcacag atacagagca tggaaatgtt |
| 4501 | tgaggaccat acaggctgta tattttggtg gattctctgt cctatacatt tacttagaag |
| 4561 | gaatggagtt atttgtctct ataaaataga cactaaaaat atttgctgaa taaatatgta |
| 4621 | ctggtca aacta |

SEQ ID NO: 3 Homo sapiens keratin 14 (KRT14), mRNA (NM_000526.5)

| | |
|---|---|
| 1 | acccgagcac cttctcttca ctcagccaac tgctcgctcg ctcacctccc tcctctgcac |
| 61 | catgaccacc tgcagccgcc agttcacctc ctccagctcc atgaagggct cctgcggcat |
| 121 | cgggggcggc atcgggggcg gctccagccg catctcctcc gtcctggccg gagggtcctg |
| 181 | ccgcgccccc agcacctacg ggggcggcct gtctgtctca tcctcccgct tctcctctgg |
| 241 | gggagcctgc gggctggggg gcggctatgg cggtggcttc agcagcagca gcagcagctt |
| 301 | tggtagtggc tttgggggag gatatggtgg tggccttggt gctggcttgg gtggtggctt |
| 361 | tggtggtggc tttgctggtg gtgatgggct tctggtgggc agtgagaagg tgaccatgca |
| 421 | gaacctcaat gaccgcctgg cctcctacct ggacaaggtg cgtgctctgg aggaggccaa |
| 481 | cgccgacctg gaagtgaaga tccgtgactg gtaccagagg cagcggcctg ctgagatcaa |
| 541 | agactacagt ccctacttca agaccattga ggacctgagg aacaagattc tcacagccac |
| 601 | agtggacaat gccaatgtcc ttctgcagat tgacaatgcc cgtctggccg cggatgactt |
| 661 | ccgcaccaag tatgagacag agttgaacct gcgcatgagt gtggaagccg acatcaatgg |
| 721 | cctgcgcagg gtgctggacg aactgaccct ggccagagct gacctggaga tgcagattga |
| 781 | gagcctgaag gaggagctgg cctacctgaa gaagaaccac gaggaggaga tgaatgccct |
| 841 | gagaggccag gtgggtggag atgtcaatgt ggagatggac gctgcacctg gcgtggacct |
| 901 | gagccgcatt ctgaacgaga tgcgtgacca gtatgagaag atggcagaga agaaccgcaa |
| 961 | ggatgccgag gaatggttct tcaccaagac agaggagctg aaccgcgagg tggccaccaa |
| 1021 | cagcgagctg gtgcagagcg gcaagagcga gatctcggag ctccggcgca ccatgcagaa |
| 1081 | cctggagatt gagctgcagt cccagctcag catgaaagca tccctggaga acagcctgga |
| 1141 | ggagaccaaa ggtcgctact gcatgcagct ggcccagatc caggagatga ttggcagcgt |
| 1201 | ggaggagcag ctggcccagc tccgctgcga gatggagcag cagaaccagg agtacaagat |
| 1261 | cctgctggac gtgaagacgc ggctggagca ggagatcgcc acctaccgcc gcctgctgga |
| 1321 | gggcgaggac gcccacctct cctcctccca gttctcctct ggatcgcagt catccagaga |
| 1381 | tgtgacctcc tccagccgcc aaatccgcac caaggtcatg gatgtgcacg atggcaaggt |
| 1441 | ggtgtccacc cacgagcagg tccttcgcac caagaactga ggctgcccag ccccgctcag |
| 1501 | gcctaggagg ccccccgtgt ggacacagat cccactggaa gatcccctct cctgcccaag |
| 1561 | cacttcacag ctggaccctg cttcaccctc accccctcct ggcaatcaat acagcttcat |
| 1621 | tatctgagtt gcataa |

SEQ ID NO: 4 Homo sapiens HP protein (Haptogloblin) mRNA, (AF026219.1)

| | |
|---|---|
| 1 | cccagccagg acatggccgc acctctcctc atcaggagcg ccggctcacg gacttctcgc |
| 61 | ccaactccct gagcgctccc tcgtttcgat ctttagaaaa ccctgctttc tttctggggc |
| 121 | cgtgacgagg ggcagggagc ggcgagcaag gatgcgttga ggaccgcgag ggcgcgcgtc |
| 181 | tcgggtgccg ccgtgggtcc cgacgcggaa gccgagccgc ctccgcctgc ctcgacttcc |
| 241 | ccacagcgct tccgccgccg cctgccgtgc ttgatgtgca gaaagaagcc ggacaccatg |
| 301 | atcctaacac aaattgaagc caaggaagct tgtgattggc tacgggcaac tggtttcccc |
| 361 | cagtatgcac agctttatga agatttcctg ttccccatcg atatttcctt ggtcaagaga |
| 421 | gagcatgatt ttttggacag agatgccatt gaggctctat gcaggcgtct aaatacttta |
| 481 | aacaaatgtg cagtgatgaa gctagaaatt agtcctcatc ggaaacgaag tgacgattca |
| 541 | gacgaggatg agccttgtgc catcagtggc aaatggactt tccaaaggga cagcaagagg |
| 601 | tggtcccggc ttgaagagtt tgatgtcttt tctccaaaac aagacctggt ccctgggtcc |
| 661 | ccagacgact cccacccgaa ggacggcccc agccccggag gcacgctgat ggacctcagc |
| 721 | gagcgccagg aggtgtcttc cgtccgcagc ctcagcagca ctggcagcct ccccagccac |
| 781 | gcgcccccca gcgaggatgc tgccaccccc cggactaact ccgtcatcag cgtttgctcc |
| 841 | tccagcaact tggcaggcaa tgacgactct ttcggcagcc tgccctctcc caaggaactg |
| 901 | tccagcttca gcttcagcat gaaaggccac gaaaaaactg ccaagtccaa gacgcgcagt |
| 961 | ctgctgaaac ggatggagag cctgaagctc aagagctccc atcacagcaa gcacaaagcg |
| 1021 | ccctcaaagc tggggttgat catcagcggg cccatcttgc aagaggggat ggatgaggag |
| 1081 | aagctgaagc agctcaactg cgtggagatc tccgccctca atggcaaccg catcaacgtc |
| 1141 | cccatggtac gaaagaggag cgtttccaac tccacgcaga ccagcagcag cagcagccag |
| 1201 | tcggagacca gcagcgcggt cagcacgccc agccctgtta cgaggacccg gagcctcagt |
| 1261 | gcgtgcaaca agcgggtggg catgtactta gagggcttcg atcctttcaa tcagtcaaca |
| 1321 | tttaacaacg tgatggagca gaactttaag aaccgcgaga gctacccaga ggacacggtg |
| 1381 | ttctacatcc ctgaagatca caagcctggc actttcccca aagctctcac caatggcagt |
| 1441 | ttctccccct cggggaataa cggctctgtg aactggagga cgggaagctt ccacggccct |
| 1501 | ggccacatca gcctcaggag ggaaaacagt agcgacagcc ccaaggaact gaagagacgc |
| 1561 | aattcttcca gctccatgag cagccgcctg agcatctacg acaacgtgcc gggctccatc |
| 1621 | ctctactcca gttcagggga cctggcggat ctggagaacg aggacatctt ccccgagctg |
| 1681 | gacgacatcc tctaccacgt gaaggggatg cagcggatag tcaatcagtg gtcggagaag |
| 1741 | ttttctgatg agggagattc ggactcagcc ctggactcgg tctctccctg cccgtcctct |
| 1801 | ccaaaacaga tacacctgga tgtggacaac gaccgaacca cacccagcga cctggacagc |
| 1861 | acaggcaact ccctgaatga accggaagag ccctccgaga tcccggaaag aagggattct |
| 1921 | ggggttgggg cttccctaac caggtccaac aggcaccgac tgagatggca cagtttccag |
| 1981 | agctcacatc ggccaagcct caactctgta tcactacaga ttaactgcca gtctgtggcc |
| 2041 | cagatgaacc tgctgcagaa atactcactc ctaaagctaa cggccctgct ggagaaatac |
| 2101 | acaccttcta acaagcatgg ttttagctgg gccgtgccca agttcatgaa gaggatcaag |
| 2161 | gttccagact acaaggaccg gagtgtgttt ggggtcccac tgacggtcaa cgtgcagcgc |
| 2221 | acaggacaac cgttgcctca gagcatccag caggccatgc gatacctccg gaaccattgt |
| 2281 | ttggatcagg ttgggctctt cagaaaatcg ggggtcaagt cccggattca ggctctgcgc |
| 2341 | cagatgaatg aaggtgccat agactgtgtc aactacgaag gacagtctgc ttatgacgtg |
| 2401 | gcagacatgc tgaagcagta ttttcgagat cttcctgagc cactaatgac gaacaaactc |
| 2461 | tcggaaacct ttctacagat ctaccaatat gtgcccaagg accagcgcct gcaggccatc |
| 2521 | aaggctgcca tcatgctgct gcctgacgag aaccgggagg ttctgcagac cctgctttat |
| 2581 | ttcctgagcg atgtcacagc agccgtaaaa gaaaaccaga tgaccccaac caacctggcc |
| 2641 | gtgtgcttag cgccttccct cttccatctc aacaccctga agagagagaa ttcctctccc |
| 2701 | agggtaatgc aaagaaaaca aagtttgggc aaaccagatc agaaagattt gaatgaaaac |
| 2761 | ctagctgcca ctcaagggct ggcccatatg atcgccgagt gcaagaagct tttccaggtt |
| 2821 | cccgaggaaa tgagccgatg tcgtaattcc tataccgaac aagagctgaa gcccctcact |
| 2881 | ctggaagcac tcgggcacct gggtaatgat gactcagctg actaccaaca cttcctccag |
| 2941 | gactgtgtgg atggcctgtt taaagaagtc aaagagaagt ttaaaggctg ggtcagctac |
| 3001 | tccacttcgg agcaggctga gctgtcctat aagaaggtga gcgaaggacc ccctctgagg |
| 3061 | ctttggaggt cagtcattga agtccctgct gtgccagagg aaatcttaaa gcgcctactt |
| 3121 | aaagaacagc acctctggga tgtagacctg ttggattcaa aagtgatcga aattctggac |
| 3181 | agccaaactg aaatttacca gtatgtccaa aacagtatgg cacctcatcc tgctcgagac |
| 3241 | tacgttgttt taagaacctg gaggactaat ttacccaaag gagcctgtgc ccttttacta |
| 3301 | acctctgtgg atcacgatcg cgcacctgtg gtgggtgtga gggttaatgt gctcttgtcc |
| 3361 | aggtatttga ttgaaccctg tgggccagga aaatccaaac tcacctacat gtgcagagtt |
| 3421 | gacttaaggg gccacatgcc agaatggtac acaaaatctt ttggacattt gtgtgcagct |
| 3481 | gaagttgtaa agatccggga ttccttcagt aaccagaaca ctgaaaccaa agacaccaaa |
| 3541 | tctaggtgat cactgaagca acgcaaccgc ttccaccacc atggtgtttg tttctagaac |
| 3601 | ttttgccagt ccttgaagaa tgggttctgt gtctaatcct gaaacaaaga aaactacaag |
| 3661 | ctggagtgta ggaattgact atagcaattt gatacatttt taaagctgct tcctgtttgt |
| 3721 | tgagggtctg tattcataga ccttgactgg aatatgtaag actgtg |

SEQ ID NO: 5 Synthetic construct Homo sapiens clone CCSBHm_00015515 CA1
(CA1) mRNA, encodes complete protein (KR710668.1)

| | |
|---|---|
| 1 | gttcgttgca acaaattgat gagcaatgct tttttataat gccaactttg tacaaaaaag |
| 61 | ttggcatggc aagtccagac tggggatatg atgacaaaaa tggtcctgaa caatggagca |
| 121 | agctgtatcc cattgccaat ggaaataacc agtcccctgt tgatattaaa accagtgaaa |
| 181 | ccaaacatga cacctctctg aaacctatta gtgtctccta caacccagcc acagccaaag |
| 241 | aaattatcaa tgtggggcat tccttccatg taaattttga ggacaacgat aaccgatcag |
| 301 | tgctgaaagg tggtcctttc tctgacagct acaggctctt tcagttccat tttcactggg |
| 361 | gcagtacaaa tgagcatggt tcagaacata cagtggatgg agtcaaatat tctgccgagc |
| 421 | ttcacgtagc tcactggaat tctgcaaagt actccagcct tgctgaagct gcctcaaagg |
| 481 | ctgatggttt ggcagttatt ggtgttttga tgaaggttgg tgaggccaac ccaaagctgc |
| 541 | agaaagtact tgatgccctc caagcaatta aaaccaaggg caaacgagcc ccattcacaa |
| 601 | attttgaccc ctctactctc cttccttcat ccctggattt ctggacctac cctggctctc |
| 661 | tgactcatcc tcctctttat gagagtgtaa cttggatcat ctgtaaggag agcatcagtg |
| 721 | tcagctcaga gcagctggca caattccgca gccttctatc aaatgttgaa ggtgataacg |
| 781 | ctgtccccat gcagcacaac aaccacccaa cccaacctct gaagggcaga acagtgagag |
| 841 | cttcattttg cccaactttc ttgtacaaag ttggcattat aagaaagcat tgcttatcaa |
| 901 | tttgttgcaa cgaac |

SEQ ID NO: 6 Homo sapiens hemoglobin subunit beta (HBB), mRNA
(NM_000518.5)

| | |
|---|---|
| 1 | acatttgctt ctgacacaac tgtgttcact agcaacctca aacagacacc atggtgcatc |
| 61 | tgactcctga ggagaagtct gccgttactg ccctgtgggg caaggtgaac gtggatgaag |
| 121 | ttggtggtga ggccctgggc aggctgctgg tggtctaccc ttggacccag aggttctttg |
| 181 | agtcctttgg ggatctgtcc actcctgatg ctgttatggg caaccctaag gtgaaggctc |
| 241 | atggcaagaa agtgctcggt gcctttagtg atggcctggc tcacctggac aacctcaagg |
| 301 | gcacctttgc cacactgagt gagctgcact gtgacaagct gcacgtggat cctgagaact |
| 361 | tcaggctcct gggcaacgtg ctggtctgtg tgctggccca tcactttggc aaagaattca |
| 421 | ccccaccagt gcaggctgcc tatcagaaag tggtggctgg tgtggctaat gccctggccc |
| 481 | acaagtatca ctaagctcgc tttcttgctg tccaatttct attaaaggtt cctttgttcc |
| 541 | ctaagtccaa ctactaaact gggggatatt atgaagggcc ttgagcatct ggattctgcc |
| 601 | taataaaaaa catttatttt cattgcaa |

SEQ ID NO: 7 Homo sapiens full open reading frame cDNA clone
RZPDo834A0126D for gene SAA1, serum amyloid A1; complete cds, without
stopcodon (CR542241.1)

| | |
|---|---|
| 1 | atgaagcttc tcacgggcct ggttttctgc tccttggtcc tgggtgtcag cagccgaagc |
| 61 | ttcttttcgt tccttggcga ggcttttgat ggggctcggg acatgtggag agcctactct |
| 121 | gacatgagag aagccaatta catcggctca gacaaatact tccatgctcg ggggaactat |
| 181 | gatgctgcca aaaggggacc tgggggtgcc tgggctgcag aagtgatcag cgatgccaga |
| 241 | gagaatatcc agagattctt tggccatggt gcggaggact cgctggctga tcaggctgcc |
| 301 | aatgaatggg gcaggagtgg caaagacccc aatcacttcc gacctgctgg cctgcctgag |
| 361 | aaatac |

SEQ ID NO: 8 Homo sapiens partial mRNA for immunoglobulin heavy chain
constant region gamma 4 (IGHG4 gene) (AJ294733.1)

| | |
|---|---|
| 1 | gcaagcttca agggcccatc ggtcttcccc ctggtgccct gctccaggag cacctccgag |
| 61 | agcacagccg ccctgggctg cctggtcaag gactacttcc ccgaaccggt gacggtgtcg |
| 121 | tggaactcat gcgccctgac cagcggcgtg cacaccttcc cggctgtcct acagtcctca |
| 181 | ggactctact ccctcagcag cgtggtgacc gtgccctcca gcagcttggg cacgaagacc |
| 241 | tacacctgca acgtagatca caagcccagc aacaccaagg tggacaagag agttgagtcc |
| 301 | aaatatggtc ccccatgccc atcatgccca gcacctgagt tcctgggggg accatcagtc |
| 361 | ttcctgttcc ccccaaaacc caaggacact ctcatgatct cccggacccc tgaggtcacg |
| 421 | tgcgtggtgg tggacgtgag ccaggaagac cccgaggtcc agttcaactg gtacgtggat |
| 481 | ggcgtggagg tgcataatgc caagacaaag ccgcgggagg agcagttcaa cagcacgtac |
| 541 | cgtgtggtca gggtcctcac cgtcctgcac caggactggc tgaacggtaa ggagtacaag |
| 601 | tgcaaggtct ccaacaaagg cctcccgtcc tccatcgaga aaaccatctc caaagccaaa |
| 661 | gggcagcccc gagagccaca ggtgtacacc ctgcccccat cccaggagga gatgaccaag |
| 721 | aaccaggtca gcctgacctg cctggtcaaa ggcttctacc ccagcgacat cgccgtggag |
| 781 | tgggagagca atgggcagcc ggaggacaac tacaagacca cgcctcccgt gctggactcc |
| 841 | gacggctcct tcttcctcta cagcaggcta accgtggaca agagcaggtg gcaggagggg |
| 901 | aatgtcttct catgctccgt gatgcatgag gctctgcaca accactacac acagaagagc |
| 961 | ctctccctgt ctccgggtaa a |

SEQ ID NO: 9 Homo sapiens phosphoribosyl pyrophosphate synthetase 1, mRNA
(cDNA clone MGC:2256 IMAGE:3542584), complete cds (BC001605.1)

| | |
|---|---|
| 1 | gcaacgcaaa gcgcttggta ttgagtctgt ggccgacttc ggttccggtc tctgcagcag |
| 61 | ccgtgatcgc ttagtggagt gcttagggta gttggccagg atgccgaata tcaaaatctt |
| 121 | cagcggcagc tcccaccagg acttatctca gaaaattgct gaccgcctgg gcctggagct |
| 181 | aggcaaggtg gtgactaaga aattcagcaa ccaggagacc tgtgtggaaa ttggtgaaag |
| 241 | tgtacgtgga gaggatgtct acattgttca gagtggttgt ggcgaaatca atgacaattt |
| 301 | aatggagctt ttgatcatga ttaatgcctg caagattgct tcagccagcc gggttactgc |
| 361 | agtcatccca tgcttccctt atgcccggca ggataagaaa gataagagcc gggcgccaat |
| 421 | ctcagccaag cttgttgcaa atatgctatc tgtagcaggt gcagatcata ttatcaccat |
| 481 | ggacctacat gcttctcaaa ttcagggctt ttttgatatc ccagtagaca atttgtatgc |
| 541 | agagccggct gtcctaaagt ggataaggga gaatatctct gagtggagga actgcactat |
| 601 | tgtctcacct gatgctggtg gagctaagag agtgacctcc attgcagaca ggctgaatgt |
| 661 | ggactttgcc ttgattcaca aagaacggaa gaaggccaat gaagtggacc gcatggtgct |
| 721 | tgtgggagat gtgaaggatc gggtggccat ccttgtggat gacatggctg acacttgtgg |
| 781 | cacaatctgc catgcagctg acaaacttct ctcagctggc gccaccagag tttatgccat |
| 841 | cttgactcat ggaatcttct ccggtcctgc tatttctcgc atcaacaacg catgctttga |
| 901 | ggcagtagta gtcaccaata ccatacctca ggaggacaag atgaagcatt gctccaaaat |
| 961 | acaggtgatt gacatctcta tgatccttgc agaagccatc aggagaactc acaatggaga |
| 1021 | atccgtttct tacctattca gccatgtccc tttataatag agtaacttct gaggcttttt |
| 1081 | gagaataaaa tccaccccac ccttgtttcc ccttggtatt tgatgacaaa ttcagcagaa |
| 1141 | gacccggctt gctccagtgt agctttctac atcccacatc aggtatatta gagcttatcc |
| 1201 | gaactgggga aagacggatt gagattaact gctgggacct cctacctgca ttatctcatt |
| 1261 | ctggcttcct tgataattct gtgggccttg cagctttaac tatagctcag ctgctgcaag |
| 1321 | atttcagact tttgaggatg ttgtgtgagg gtgtttgact gtgactgggg aagctcagac |
| 1381 | tactttgtat gtgaatgctt cagggttttc tttgttgaga acaactagca acaaaggcaa |
| 1441 | cccatgtgtg accagttctc cccaaggtct atgctaaatt atagcaagag ccctgggcaa |
| 1501 | ccccaaacct agtcctggta gctgagcacc ctgtaaggca ggagcaggca gctcagcttg |
| 1561 | agcagacatt gggtgggggg tggggggtgg ttgagggggg aggcagcaca gtgcagcaaa |
| 1621 | tgtttcttgg gaggaagaag cctgatccat caccatctgc ttgactatgt agcttggatt |
| 1681 | ctcctttgta cctatccctt tcgatttggc tttaccttca tctatcttga tcctttcctg |
| 1741 | gccaaatatc ctcttgggcc caaatgaaca ttgtaccata gtcttctgga aagcaaacat |
| 1801 | gcttcctgct atgtaattgc taacattcat attagatgat gtgctgtagc ttgatcttcc |
| 1861 | ttagcctact gccactgagg cagtaggttt taggtggtat cgtagtgcct tttgattaat |
| 1921 | ttaagtattt aattttcatc ttccttcttt ggatctattt ggcctctcaa atgaactgag |
| 1981 | attcctgtta aaaaagattg atgttattgt ctcttgtaga ggaaactaat aaagtgtgtg |
| 2041 | tacctgtgtg aaaaaaaaaa aaaaaaaaaa aaaaaaaa |

SEQ ID NO: 10 Synthetic construct Homo sapiens clone ccsbBroadEn_03028
RBMX gene, encodes complete protein (KJ893634.1)

| | |
|---|---|
| 1 | gttcgttgca acaaattgat gagcaatgct tttttataat gccaactttg tacaaaaaag |
| 61 | ttggcatggt tgaagcagat cgcccaggaa agctcttcat tggtgggctt aatacggaaa |
| 121 | caaatgagaa agctcttgaa gcagtatttg gcaaatatgg acgaatagtg gaagtactct |
| 181 | tgatgaaaga ccgtgaaacc aacaaatcaa gaggatttgc ttttgtcacc tttgaaagcc |
| 241 | cagcagacgc taaggatgca gccagagaca tgaatggaaa gtcattagat ggaaaagcca |
| 301 | tcaaggtgga acaagccacc aaaccatcat ttgaaagtgg tagacgtgga ccgcctccac |
| 361 | ctccaagaag tagaggccct ccaagaggtc ttagaggtgg aagaggagga agtggaggaa |
| 421 | ccaggggacc tccctcacgg ggaggacaca tggatgacgg tggatattcc atgaatttta |
| 481 | acatgagttc ttccagggga ccactcccag taaaaagagg accaccacca agaagtgggg |
| 541 | gtcctcctcc taagagatct gcaccttcag gaccagttcg cagtagcagt ggaatgggag |
| 601 | gaagagctcc tgtatcacgt ggaagagata gttatggagg tccacctcga agggaaccgc |
| 661 | tgccctctcg tagagatgtt tatttgtccc caagagatga tgggtattct actaaagaca |
| 721 | gctattcaag cagagattac ccaagttctc gtgatactag agattatgca ccaccaccac |
| 781 | gagattatac ttaccgtgat tatggtcatt ccagttcacg tgatgactat ccatcaagag |
| 841 | gatatagcga tagagatgga tatggtcgtg atcgtgacta ttcagatcat ccaagtggag |
| 901 | gttcctacag agattcatat gagagttatg gtaactcacg tagtgctcca cctacacgag |
| 961 | ggcccccgcc atcttatggt ggaagcagtc gctatgatga ttacagcagc tcacgtgacg |
| 1021 | gatatggtgg aagtcgagac agttactcaa gcagccgaag tgatctctac tcaagtggtc |
| 1081 | gtgatcgggt tggcagacaa gaaagagggc ttcccccttc tatggaaagg gggtaccctc |
| 1141 | ctccacgtga ttcctacagc agttcaagcc gcggagcacc aagaggtggt ggccgtggag |
| 1201 | gaagccgatc tgatagaggg ggaggcagaa gcagatacta cccaactttc ttgtacaaag |
| 1261 | ttggcattat aagaaagcat tgcttatcaa tttgttgcaa cgaac |

SEQ ID NO: 11 NSF Homo sapiens N-ethylmaleimide-sensitive factor (NSF)
mRNA, complete cds (AF135168.1)

| | |
|---|---|
| 1 | gcgggggcgg gctttgccga gcgcagagct gcagccgccg agccggacgt gtgcgcgaag |
| 61 | atggcgggcc ggagcatgca agcggcaaga tgtcctacag atgaattatc tttaaccaat |
| 121 | tgttcagttg tgaatgaaaa ggatttccag tctggccagc atgtgattgt gaggacctct |
| 181 | cccaatcaca ggtacacatt tacactgaag acacatccat cggtggttcc agggagcatt |
| 241 | gcattcagtt tacctcagag aaaatgggct gggctttcta ttgggcaaga aatagaagtc |
| 301 | tccttatata catttgacaa agccaaacag tgtattggca caatgaccat cgagattgat |
| 361 | ttcctgcaga aaaaaagcaa tgactccaac ccttatgaca ccgacaagat ggcagcagaa |
| 421 | tttattcagc aattcaacaa ccaggcctac tcagtgggac aacagcttgt ctttagcttc |
| 481 | aatgaaaagc tttttggctt actggtgaag gacattgaat ccatggatcc tagcatcctg |
| 541 | aagggagagc ctgcgacagg gaaaaggcag aagattgaag taggactggt tgttggaaac |
| 601 | agtcaagttg catttgaaaa agcagaaaat tcgtcactta atcttattgg caaagctaaa |
| 661 | accaaggaaa atcgccaatc aattatcaat cctgactgga actttgaaaa aatgggaata |
| 721 | ggaggtctag acaaggaatt ttcagatatt ttccgacgag catttgcctt ccgagtattt |
| 781 | cctccagaga ttgtggagca gatgggttgt atacatgtta aaggcatcct gttatatgga |
| 841 | cccccaggtt gtggtaagac tctcttggct cgacagattg gcaagatgtt gaatgcaaga |
| 901 | gagcccaaag tggtcaatgg gccagaaatc cttaacaaat atgtgggaga atcagaggct |
| 961 | aacattcgca aactttttgc tgatgctgaa gaggagcaaa ggaggcttgg tgctaacagt |
| 1021 | ggtttgcaca tcatcatctt tgatgaaatt gatgccatct gcaagcagag agggagcatg |
| 1081 | gctggtagca cgggagttca tgacactgtt gtcaaccagt tgctgtccaa aattgatggc |
| 1141 | gtggagcagc taaacaacat cctagtcatt ggaatgacca atagaccaga tctgatagat |
| 1201 | gaggctcttc ttagacctgg aagactggaa gttaaaatgg agataggctt gccagatgag |
| 1261 | aaaggccgac tacagattct tcacatccac acagcaagaa tgagagggca tcagttactc |
| 1321 | tctgctgatg tagacattaa agaactggcc gtggagacca agaatttcag tggtgctgaa |
| 1381 | ttggagggtc tggtgcgagc agcccagtcc actgctatga atagacacat aaaggccagt |
| 1441 | actaaagtgg aagtggacat ggagaaagca gaaagcctgc aagtgacgag aggagacttc |
| 1501 | cttgcttctt tggagaatga tatcaaacca gcctttggca caaaccaaga agattatgca |
| 1561 | agttacatta tgaacggtat catcaaatgg ggtgacccag ttactcgagt tctagatgat |
| 1621 | ggggagctgc tggtgcagca gactaagaac agtgaccgca caccattggt cagcgtgctt |
| 1681 | ctggaaggcc ctcctcacag tgggaagact gctttagctg caaaaattgc agaggaatcc |
| 1741 | aacttcccgt tcatcaagat ctgttctcct gataaaatga ttggcttttc tgaaacagcc |
| 1801 | aaatgtcagg ccatgaagaa gatctttgat gatgcgtaca aatcccagct cagttgtgtg |
| 1861 | gttgtggatg acattgagag attgcttgat tacgtcccta ttggccctcg attttcaaat |
| 1921 | cttgtattac aggctcttct cgttttactg aaaaaggcac ctcctcaggg ccgcaagctt |
| 1981 | cttatcattg ggaccactag ccgcaaagat gtccttcagg agatggaaat gcttaacgct |
| 2041 | ttcagcacca ccatccacgt gcccaacatt gccacaggag agcagctgtt ggaagctttg |
| 2101 | gagcttttgg gcaacttaaa ggataaggaa cgcaccacaa ttgcacagca agtcaaaggg |
| 2161 | aagaaggtct ggataggaat caagaagtta ctaatgctga tcgagatgtc cctacagatg |
| 2221 | gatcctgaat accgtgtgag aaaattcttg gccctcttaa gagaagaagg agctagcccc |
| 2281 | cttgattttg attgaaaatg aactatttga aacacacagt gaccaaggga agtgaccaag |
| 2341 | gtgaagatgg cctaggatct tcactgtctt actcaagata ctggactaag tggaacgttc |
| 2401 | tctaccttca acatgtgctc gctctgcatg attagtgcaa taaaactccc ttccttatgc |
| 2461 | atactgagat agcttagtgt ctcgtggaag gtgtcaattt ggtttagaat gctgcgctta |
| 2521 | ccttcccatg caggctaaag tgattccttc ttgctcagtc cctctgggtg ggaaccatcc |
| 2581 | agtacttgtg gacactacac gtttcaacct ctctactagc accatcaccc ttgaaaactc |
| 2641 | tcagtcagtg tcatgaatgt tgcatgacaa cagttggccg attagaaggc agactttcta |
| 2701 | catgcaaatc tggcttagta aatcgaggtg tgggccagag atcctctgac agctgtcctg |
| 2761 | agctaacact aaaagtcact gggtatttgg ttaaaggtct cccacaagac tggtattctc |
| 2821 | tttgcctgaa gaaacaaggc attgaatctc taaaatgctg ttctcaatca ttgtcagaga |
| 2881 | tgttttcaag ttgcagtcag aagatctttc ttaatagaaa gtcagatgac taccgtgttg |
| 2941 | gttgtgactt ccccttaagt ataactaatt tgctctgtgg taagagatat gctcattatt |
| 3001 | accacttaga agatgttgtt aaaaacatgt gaaagatagg tatggaaaaa gcatacaccc |
| 3061 | ccaaacagaa aggagttatt aaagtaattt acaaacctct cagcactaat tagtgtccaa |
| 3121 | ctccaagtgg gtcaattcct tagtataata ttaaggctta ctagtatcac tgctttttcc |
| 3181 | ttagcttaat gacttactta gaatttatcc tttattttaa atgatctgta ctatctagtg |
| 3241 | tctaaaacac tattctccag aaaaatcaat cattttctag ccctctccct cagtccttta |
| 3301 | ttgtccattc caatacattg aacacatttc ctttaccctc cacacacttc ttccaaaagg |
| 3361 | aagcacccgt tgagtccttt tgagggtgat ttgtcttaca actgactgac ttagcaggaa |
| 3421 | tttaattagg tcatatttgg tgatgagact tatggagtgt gcctctctct cccaactgct |
| 3481 | gcttaaaatg caaggacaag caattagaag ccatcctaag gtgcttacct cacacgccac |
| 3541 | ccatgaggct tgtggccaca gtggcacttg ggtgtggctc ctctgttatt tgtcctcatg |
| 3601 | tgagaaagca gatcatctcc aaatcttgcc atttgtatac ttttggtgga gacttggatg |
| 3661 | tcatatcttc tttgttttgg gttttcttcc ctagcttatt ttgtggcttt taaagaagtg |
| 3721 | gattgtattg tgagatcctg tgattcctgg tggccagtat cctggattcc tctaagatct |
| 3781 | tgcctctttc ctcctcatga aagcagcaca cattgtgtta acttatgtct cttgttaaat |
| 3841 | gagcttaatg tctttgtgtt ttgtccaaaa ctgtattgaa aaaatattgt ttaatgcaaa |
| 3901 | tgaaggaatg caataaagag taaatatact tgaaaaaaaa aaaaaaaaaa aaaaaaaaaa |

SEQ ID NO: 12 Homo sapiens isolate P1177K immunoglobulin kappa light chain
variable region (IGKV2D-29) mRNA, partial cds

| | |
|---|---|
| 1 | gtgatgaccc agactccact ctctctgtcc gtcacccctg gacagccggc ctccatctcc |
| 61 | tgcaagtcta gtcagagcct cctgcatagt gatggaaaga cctatttgta ttggtacctg |
| 121 | cagaagccag gccagtctcc acagctcctg atctatgaag tttccaaccg gttctctgga |
| 181 | gtgccagata ggttcagtgg cagcgggtca gggacagatt tcacactgaa aatcagccgg |
| 241 | gtggaggctg aggatgttgg ggtttattac tgcatgcaaa gtatacagcc ttacactttt |
| 301 | ggccagggga ccaaggtgga gatcaaac |

SEQ ID NO: 13 Homo sapiens progesterone receptor membrane component 1 (1),
mRNA (cDNA clone MGC:8891 IMAGE:3863377), complete cds

| | |
|---|---|
| 1 | gtggcgagtt ccggatccct gcctagcgcg gcccaacctt tactccagag atcatggctg |
| 61 | ccgaggatgt ggtggcgact ggcgccgacc caagcgatct ggagagcggc gggctgctgc |
| 121 | atgagatttt cacgtcgccg ctcaacctgc tgctgcttgg cctctgcatc ttcctgctct |
| 181 | acaagatcgt gcgcggggac cagccggcgg ccagcggcga cagcgacgac gacgagccgc |
| 241 | cccctctgcc ccgcctcaag cggcgcgact tcacccccgc cgagctgcgg cgcttcgacg |
| 301 | gcgtccagga cccgcgcata ctcatggcca tcaacggcaa ggtgttcgat gtgaccaaag |
| 361 | gccgcaaatt ctacgggccc gaggggccgt atggggtctt tgctggaaga gatgcatcca |
| 421 | ggggccttgc cacattttgc ctggataagg aagcactgaa ggatgagtac gatgaccttt |
| 481 | ctgacctcac tgctgcccag caggagactc tgagtgactg ggagtctcag ttcactttca |
| 541 | agtatcatca cgtgggcaaa ctgctgaagg agggggagga gcccactgtg tactcagatg |
| 601 | aggaagaacc aaaagatgag agtgcccgga aaaatgatta aagcattcag tggaagtata |
| 661 | tctatttttg tattttgcaa aatcatttgt aacagtccac tctgtcttta aaacatagtg |
| 721 | attacaatat ttagaaagtt ttgagcactt gctataagtt ttttaattaa catcactagt |
| 781 | gacactaata aaattaactt cttagaatgc atgatgtgtt tgtgtgtcac aaatccagaa |
| 841 | agtgaactgc agtgctgtaa tacacatgtt aatactgttt ttcttctatc tgtagttagt |
| 901 | acaggatgaa tttaaatgtg tttttcctga gagacaagga agacttgggt atttcccaaa |
| 961 | acaggtaaaa atcttaaatg tgcaccaaga gcaaaggatc aacttttagt catgatgttc |
| 1021 | tgtaaagaca acaaatccct ttttttttct caattgactt aactgcatga tttctgtttt |
| 1081 | atctacctct aaagcaaatc tgcagtgttc caaagacttt ggtatggatt aagcgctgtc |
| 1141 | cagtaacaaa atgaaatctc aaaacagagc tcagctgcaa aaaagcatat tttctgtgtt |
| 1201 | tctggactgc actgttgtcc ttgccctcac atagacactc agacaccctc acaaacacag |
| 1261 | tagtctatag ttaggattaa aataggatct gaacattcaa aagaaagctt tggaaaaaaa |
| 1321 | gagctggctg gcctaaaaac ctaaatatat gatgaagatt gtaggactgt cttcccaagc |
| 1381 | cccatgttca tggtggggca atggttattt ggttatttta ctcaattggt tactctcatt |
| 1441 | tgaaatgagg gagggacata cagaatagga acaggtgttt gctctcctaa gagccttcat |
| 1501 | gcacacccct gaaccacgag gaaacagtac agtcgctagt caagtggttt ttaaagtaaa |
| 1561 | gtatattcat aaggtaacag ttattctgtt gttataaaac tatacccact gcaaaagtag |
| 1621 | tagtcaagtg tctaggtctt tgatattgct cttttggtta acactaagct taagtagact |
| 1681 | atacagttgt atgaatttgt aaaagtatat gaacacctag tgagatttca aacttgtaat |
| 1741 | tgtggttaaa tagtcattgt attttcttgt gaactgtgtt ttatgatttt acctcaaatc |
| 1801 | agaaaacaaa atgatgtgct ttggtcagtt aataaaaatg gttttaccca caaaaaaaaa |
| 1861 | aaaaaa |

TABLE 2

| Gene | Gene Accession |
|---|---|
| KRT1 | NG_008364.1 |
| PZP | NG_052998.1 |
| KRT14 | NG_008624.1 |
| HP | NG_012651.1 |
| CA1 | NG_016221.1 |
| HBB | NG_059281.1 |
| SAA1 | NG_021330.1 |
| IGHG4 | NG_001019.6 |
| PRPS1 | NG_008407.1 |
| RBMX | NG_012918.1 |
| NSF | AY413895.1 |
| IGKV2D-29 | NG_000833.2 |
| PGRMC1 | NG_016756.1 |

TABLE 3

| Uniprot Accession | Gene |
|---|---|
| P04264 | KRT1 |
| P20742 | PZP |
| P02533 | KRT14 |
| H0Y300 | HP |
| P00915 | CA1 |
| P68871 | HBB |
| P0DJI8 | SAA1 |
| A0A286YFJ8 | IGHG4 |
| P60891 | PRPS1 |
| P38159 | RBMX |
| P46459 | NSF |
| A0A075B6S2 | IGKV2D-29 |
| O00264 | PGRMC1 |

Methods

Described herein are diagnostic methods, assays, and systems for endometriosis and related conditions (e.g., endometriosis cysts and ovarian cancer).

Provided herein are methods of determining whether a subject has an endometriosis-related condition. In some embodiments, the methods comprise measuring the levels of or activity of at least one protein (e.g., at least two, at least three, at least four, at least five, at least six, at least seven, at least eight, at least nine, at least ten, at least eleven, at least twelve, at least thirteen, at least fourteen, or at least fifteen) encoded by at least one gene (e.g., at least two, at least three, at least four, at least five, at least six, at least seven, at least eight, at least nine, at least ten, at least eleven, at least twelve, or all thirteen) selected from KRT1, PZP, KRT14, HP, CA1, HBB, SAA1, IGHG4, PRPS1, RBMX, NSF, IGKV2D-29, and PGRMC1 in a biological sample isolated from the subject, and if the level of or activity of the protein(s) is elevated compared to a level or activity of the protein(s) in a biological sample from a subject who is not afflicted with an endometrioses-related condition, the subject is determined to have the endometrioses-related condition. For example, the methods provided herein may comprise measuring the levels of or activity of at least two proteins, wherein each proteins is encoded by a different gene (e.g., different genes disclosed herein).

The methods may comprise measuring the levels of or activity of at least one protein (e.g., at least two, at least three, at least four, or at least five proteins) encoded by KRT1. The methods may comprise measuring the levels of or activity of at least one protein (e.g., at least two, at least three, at least four, or at least five proteins) encoded by PZP. The methods may comprise measuring the levels of or activity of at least one protein (e.g., at least two, at least three, at least four, or at least five proteins) encoded by KRT14. The methods may comprise measuring the levels of or activity of at least one protein (e.g., at least two, at least three, at least four, or at least five proteins) encoded by HP. The methods may comprise measuring the levels of or activity of at least one protein (e.g., at least two, at least three, at least four, or at least five proteins) encoded by CA. The methods may comprise measuring the levels of or activity of at least one protein (e.g., at least two, at least three, at least four, or at least five proteins) encoded by HBB. The methods may comprise measuring the levels of or activity of at least one protein (e.g., at least two, at least three, at least four, or at least five proteins) encoded by SAA1. The methods may comprise measuring the levels of or activity of at least one protein (e.g., at least two, at least three, at least four, or at least five proteins) encoded by IGHG4. The methods may comprise measuring the levels of or activity of at least one protein (e.g., at least two, at least three, at least four, or at least five proteins) encoded by PRPS1. The methods may comprise measuring the levels of or activity of at least one protein (e.g., at least two, at least three, at least four, or at least five proteins) encoded by RBMX. The methods may comprise measuring the levels of or activity of at least one protein (e.g., at least two, at least three, at least four, or at least five proteins) encoded by NSF. The methods may comprise measuring the levels of or activity of at least one protein (e.g., at least two, at least three, at least four, or at least five proteins) encoded by IGKV2D-29. The methods may comprise measuring the levels of or activity of at least one protein (e.g., at least two, at least three, at least four, or at least five proteins) encoded by PGRMC1.

The levels or activity of the at least one protein(s) may be measured by any technique known in the art, including but not limited to contacting the biological sample with an antibody specific for the at least one protein(s).

The methods described herein also comprise administering to the subject a therapy for the endometriosis-related condition if the subject is determined to have an endometriosis related condition.

In some aspects, provided herein are methods of determining whether a subject has endometriosis-related condition by measuring the levels of an at least one RNA product (e.g., at least two, at least three, at least four, at least five, at least six, at least seven, at least eight, at least nine, at least ten, at least eleven, at least twelve, at least thirteen, at least fourteen, or at least fifteen) of at least one gene (e.g., at least two, at least three, at least four, at least five, at least six, at least seven, at least eight, at least nine, at least ten, at least eleven, at least twelve, or all thirteen) selected from KRT1, PZP, KRT14, HP, CA1, HBB, SAA1, IGHG4, PRPS1, RBMX, NSF, IGKV2D-29, and PGRMC1 in the subject. In some embodiments, if the level of the at least one RNA product is elevated compared to a level of the RNA product in a subject who is not afflicted with an endometrioses-related condition, the subject is determined to have an endometrioses-related condition.

The methods may comprise measuring the levels of or activity of at least one RNA product (e.g., at least two, at least three, at least four, or at least five RNA products) encoded by KRT1. The methods may comprise measuring the levels of or activity of at least one RNA product (e.g., at least two, at least three, at least four, or at least five RNA products) encoded by PZP. The methods may comprise measuring the levels of or activity of at least one RNA product (e.g., at least two, at least three, at least four, or at least five RNA products) encoded by KRT14. The methods may comprise measuring the levels of or activity of at least one RNA product (e.g., at least two, at least three, at least four, or at least five RNA products) encoded by HP. The methods may comprise measuring the levels of or activity of at least one RNA product (e.g., at least two, at least three, at least four, or at least five RNA products) encoded by CA1. The methods may comprise measuring the levels of or activity of at least one RNA product (e.g., at least two, at least three, at least four, or at least five RNA products) encoded by HBB. The methods may comprise measuring the levels of or activity of at least one RNA product (e.g., at least two, at least three, at least four, or at least five RNA products) encoded by SAA1. The methods may comprise measuring the levels of or activity of at least one RNA product (e.g., at least two, at least three, at least four, or at least five RNA products) encoded by IGHG4. The methods may comprise measuring the levels of or activity of at least one RNA product (e.g., at least two, at least three, at least four, or at least five RNA products) encoded by PRPS1. The methods may comprise measuring the levels of or activity of at least one RNA product (e.g., at least two, at least three, at least four, or at least five RNA products) encoded by RBMX. The methods may comprise measuring the levels of or activity of at least one RNA product (e.g., at least two, at least three, at least four, or at least five RNA products) encoded by NSF. The methods may comprise measuring the levels of or activity of at least one RNA product (e.g., at least two, at least three, at least four, or at least five RNA products) encoded by IGKV2D-29. The methods may comprise measuring the levels of or activity of at least one RNA product (e.g., at least two, at least three, at least four, or at least five RNA products) encoded by PGRMC1.

Also provided herein are methods of treating or preventing an endometriosis-related condition in a subject in need thereof, by measuring the levels of or activity of at least one protein encoded by at least one gene selected from KRT1, PZP, KRT14, HP, CA1, HBB, SAA), IGHG4, PRPS1, RBMX, NSF, IGKV2D-29, and PGRMC1 in a biological sample isolated from the subject, and if the level of or activity of the protein(s) is elevated compared to a level or activity of the protein(s) in a biological sample from a subject who is not afflicted with an endometrioses-related condition, administering to the subject a therapy for the endometriosis-related condition.

In some aspects, provided herein are methods of treating or preventing an endometriosis-related condition in a subject in need thereof by measuring the levels of an at least one RNA product of at least one gene selected from KRT1, PZP, KRT14, HP, CA1, HBB, SAA1, IGHG4, PRPS1, RBMX, NSF, IGKV2D-29, and PGRMC1 in the subject, and if the level of the at least one RNA product(s) is elevated compared to a level of the RNA product(s) in a subject who is not afflicted with an endometrioses-related condition, administering to the subject at least one therapy for the endometriosis-related condition.

The therapy may be any therapy administered to a subject afflicted with an endometriosis-related condition. The therapy may be dependent on the endometriosis-related condition. For example, if the endometriosis-related condition is endometriosis, then the therapy may be hormonal contraceptives, such as birth control pills, patches and vaginal rings help control the hormones responsible for the buildup of endometrial tissue each month. The therapy may be gonadotropin-releasing hormone (Gn-RH) agonists and antagonists. These drugs block the production of ovarian-stimulating hormones, lowering estrogen levels and preventing menstruation. The therapy may be progestin therapy. Progestin therapies include intrauterine device with levonorgestrel, contraceptive implant, contraceptive injection, or a progestin pill. The therapy may be aromatase inhibitors. Aromatase inhibitors are a class of medicines that reduce the amount of estrogen in the body. The therapy may be any therapy that halts menstral cycles.

In some embodiments, the subject has multiple endometriosis-related conditions, such as endometriod or ovarian cancer. In these cases, the subject may be administered any anti-cancer therapeutic. Non-limiting examples of therapeutic agents include anti-cancer therapeutics. Non-limiting examples of anti-cancer therapeutics can include gemcitabine, cisplatin, paclitaxel, carboplatin, bortezomib, AMG479, vorinostat, rituximab, temozolomide, rapamycin, ABT-737, PI-103; alkylating agents such as thiotepa and CYTOXAN® cyclosphosphamide; alkyl sulfonates such as busulfan, improsulfan and piposulfan; aziridines such as benzodopa, caroquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin: duocarmycin (including the synthetic analogues, KW-2189 and CB1-TM1); eleutherobin; pancratistatin; a sarcodictyin; spongistatin: nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard nitrosureas such as carmustine, chlorozotocin, fotemustine, lomustine, nimustine, and ranimnustine; antibiotics such as the enediyne antibiotics (e.g., calicheamicin, especially calicheamicin gamma1I and calicheamicin omegaI1 (see, e.g., Agnew, Chem. Intl. Ed. Engl., 33: 183-186 (1994)); dynemicin, including dynemicin A: bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin. 6-diazo-5-oxo-L-norleucine. ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone: anti-adrenals such as aminoglutethimide, mitotane, trilostane; folic acid replenisher such as frolinic acid; aceglatone; aldophosphamide glycoside; aminolevulinic acid; eniluracil: amsacrine; bestrabucil; bisantrene; edatraxate; defofamine: demecolcine; diaziquone; elformithine: elliptinium acetate; an epothilone; etoglucid; gallium nitrate; hydroxyurea; lentinan; lonidainine; maytansinoids such as maytansine and ansamitocins; mitoguazone: mitoxantrone; mopidanmol; nitraerine: pentostatin; phenamet; pirarubicin; losoxantrone; podophyllinic acid; 2-ethylhydrazide; procarbazine: PSK® polysaccharide complex (JHS Natural Products. Eugene, Oreg.): razoxane: rhizoxin; sizofuran; spirogermanium; tenuazonic acid; triaziquone: 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa: taxoids, e.g., TAXOL® paclitaxel (Bristol-Myers Squibb Oncology, Princeton. N.J.), ABRAXANE® Cremophor-free, albumin-engineered nanoparticle formulation of paclitaxel (American Pharmaceutical Partners, Schaumberg. Ill.), and TAXOTERE® doxetaxel (Rhone-Poulene Rorer, Antony. France); chloranbucil; GEMZAR® gemcitabine; 6-thioguanine; mercaptopurine; methotrexate; platinum analogs such as cisplatin, oxaliplatin and carboplatin; vinblastine; platinum: etoposide (VP-16); ifosfamide; mitoxantrone: vincristine; NAVE LBINE™ vinorelbine; novantrone; teniposide; edatrexate; daunomycin; aminopterin; xeloda; ibandronate: irinotecan (Camptosar. CPT-11) (including the treatment regimen of irinotecan with 5-FU and leucovorin); topoisomerase inhibitor RFS 2000; difluoromethylornithine (DMFO); retinoids such as retinoic acid: capecitabine; combretastatin; leucovorin (LV); oxaliplatin, including the oxaliplatin treatment regimen (FOLFOX); lapatinib (Tykerb™); inhibitors of PKC-alpha, Raf, II-Ras, EGFR (e.g., erlotinib (Tarceva®)), immune checkpoint inhibitors, and VEGF-A that reduce cell proliferation and pharmaceutically acceptable salts, acids or derivatives of any of the above.

The biological sample may be any tissue that would show expression of a product of a gene disclosed herein. The biological sample or tissue may be endometrial tissue, cervical tissue, cervical cells, or uterine tissue. The endometriosis-related condition may be, for example, endometriosis, endometriosis cysts, endometrioid cancer, or ovarian cancer.

### Sample Collection, Preparation and Separation

In some embodiments, biomarker amount and/or activity measurement(s) in a sample from a subject is compared to a predetermined control (standard) sample. The sample from the subject is typically from a diseased tissue, such as tissue affected by endometriosis. The control sample can be from the same subject or from a different subject. The control sample is typically a normal, non-diseased sample. However, in some embodiments, such as for staging of disease or for evaluating the efficacy of treatment, the control sample can be from a diseased tissue. The control sample can be a combination of samples from several different subjects. In some embodiments, the biomarker amount and/or activity measurement(s) from a subject is compared to a pre-determined level. This pre-determined level is typically obtained from normal samples. As described herein, a “pre-determined” biomarker amount and/or activity measurement(s) may be a biomarker amount and/or activity measurement(s) used to, by way of example only, evaluate a subject that may be selected for treatment, evaluate a response to a therapy for endometriosis or an endometriosis related condition. A pre-determined biomarker amount and/or activity measurement(s) may be determined in populations of patients with or without endometriosis. The pre-determined biomarker amount and/or activity measurement(s) can be a single number, equally applicable to every patient, or the pre-determined biomarker amount and/or activity measurement(s) can vary according to specific subpopulations of patients. Age, weight, height, and other factors of a subject may affect the pre-determined biomarker amount and/or activity measurement(s) of the individual. Furthermore, the pre-determined biomarker amount and/or activity can be determined for each subject individually. In one embodiment, the amounts determined and/or compared in a method described herein are based on absolute measurements.

In one embodiment, the amounts determined and/or compared in a method described herein are based on relative measurements, such as ratios (e.g., protein level, expression level, and/or activity before a treatment vs. after a treatment, such biomarker measurements relative to a spiked or man-made control, such biomarker measurements relative to the expression of a housekeeping gene, and the like). For example, the relative analysis can be based on the ratio of pre-treatment biomarker measurement as compared to post-treatment biomarker measurement. Pre-treatment biomarker measurement can be made at any time prior to initiation of therapy. Post-treatment biomarker measurement can be made at any time after initiation of therapy. In some embodiments, post-treatment biomarker measurements are made 1, 2, 3, 4, 5, 6, 7, 8, 9, 10, 11, 12, 13, 14, 15, 16, 17, 18, 19, 20 weeks or more after initiation of therapy, and even longer toward indefinitely for continued monitoring.

The pre-determined biomarker amount and/or activity measurement(s) can be any suitable standard. For example, the pre-determined biomarker amount and/or activity measurement(s) can be obtained from the same or a different human for whom a patient selection is being assessed. In one embodiment, the pre-determined biomarker amount and/or activity measurement s) can be obtained from a previous assessment of the same patient. In such a manner, the progress of the selection of the patient can be monitored over time. In addition, the control can be obtained from an assessment of another human or multiple humans, e.g., selected groups of humans, if the subject is a human. In such a manner, the extent of the selection of the human for whom selection is being assessed can be compared to suitable other humans, e.g., other humans who are in a similar situation to the human of interest, such as those suffering from similar or the same condition(s) and/or of the same ethnic group.

Provided herein are methods of monitoring the progression of an endometriosis-related condition in a subject. In some embodiments, the methods comprise measuring the levels of or activity of at least one protein (e.g., at least two, at least three, at least four, at least five, at least six, at least seven, at least eight, at least nine, at least ten, at least eleven, at least twelve, at least thirteen, at least fourteen, or at least fifteen proteins) encoded by at least one gene (e.g., at least two, at least three, at least four, at least five, at least six, at least seven, at least eight, at least nine, at least ten, at least eleven, at least twelve, or all thirteen) selected from KRT1, PZP, KRT14, HP, CA1, HBB, SAA1, IGHG4, PRPS1, RBMX, NSF, IGKV2D-29, and PGRMC1 in a first biological sample isolated from the subject, and, after a period of time, measuring the levels of or activity of at least one protein (e.g., at least two, at least three, at least four, at least five, at least six, at least seven, at least eight, at least nine, at least ten, at least eleven, at least twelve, at least thirteen, at least fourteen, or at least fifteen proteins) encoded by at least one gene (e.g., at least two, at least three, at least four, at least five, at least six, at least seven, at least eight, at least nine, at least ten, at least eleven, at least twelve, or all thirteen) selected from KRT1, PZP, KRT14, HP, CA1, HBB, SAA1, IGHG4, PRPS1, RBMX, NSF, IGKV2D-29, and PGRMC1 in a second biological sample isolated from the subject. The methods further comprise determining that the condition has progressed if the level of or activity of the protein(s) in the second biological sample is elevated or higher compared to a level or activity of the protein(s) in the first biological sample from a subject. Alternatively, the methods may further comprise determining that the condition has not progressed if the level of or activity of the protein(s) in the second biological sample is the same or decreased compared to a level or activity of the protein(s) in the first biological sample from a subject.

In some embodiments, measuring or monitoring the progression of a condition disclosed herein in a subject includes measuring the levels of an at least one RNA product (e.g., at least two, at least three, at least four, at least five, at least six, at least seven, at least eight, at least nine, at least ten, at least eleven, at least twelve, at least thirteen, at least fourteen, or at least fifteen RNA products) of at least one gene (e.g., at least two, at least three, at least four, at least five, at least six, at least seven, at least eight, at least nine, at least ten, at least eleven, at least twelve, or all thirteen) selected from KRT1, PZP, KRT14, HP, CA1, HBB, SAA), IGHG4, PRPS1, RBMX, NSF, IGKV2D-29, and PGRMC1 in a first biological sample from the subject, and after a period of time, measuring the levels of an at least one RNA product (e.g., at least two, at least three, at least four, at least five, at least six, at least seven, at least eight, at least nine, at least ten, at least eleven, at least twelve, at least thirteen, at least fourteen, or at least fifteen RNA products) of at least one gene (e.g., at least two, at least three, at least four, at least five, at least six, at least seven, at least eight, at least nine, at least ten, at least eleven, at least twelve, or all thirteen) selected from KRT1, PZP, KRT14, HP, CA1, HBB, SAA1, IGHG4, PRPS1, RBMX, NSF, IGKV2D-29, and PGRMC1 in a second biological sample fro the subject. In some embodiments, the disease is determined to have progressed if the level of the at least one RNA product in the first biological sample is elevated compared to a level of the RNA product in a second biological sample from the subject. In some embodiments, the disease is determined to not have progressed if the level of the at least one RNA product in the first biological sample is the same or decreased compared to a level of the RNA product in a second biological sample from the subject.

The period of time may be at least one hour, at least six hours, at least 24 hours, at least one week, at least one month, at least three months, or at least six months.

In some embodiments of the present invention the change of biomarker amount and/or activity measurement(s) from the pre-determined level is about 0.1, 0.2, 0.3, 0.4, 0.5, 0.6, 0.7, 0.8, 0.9, 1.0, 1.5, 2.0, 2.5, 3.0, 3.5, 4.0, 4.5, or 5.0 fold or greater, or any range in between, inclusive. Such cutoff values apply equally when the measurement is based on relative changes, such as based on the ratio of pre-treatment biomarker measurement as compared to post-treatment biomarker measurement.

Biological samples can be collected from a variety of sources from a patient including a body fluid sample, cell sample, or a tissue sample comprising nucleic acids and/or proteins. “Body fluids” refer to fluids that are excreted or secreted from the body as well as fluids that are normally not (e.g., amniotic fluid, aqueous humor, bile, blood and blood plasma, cerebrospinal fluid, cerumen and earwax, cowper's fluid or pre-ejaculatory fluid, chyle, chyme, stool, female ejaculate, interstitial fluid, intracellular fluid, lymph, menses, breast milk, mucus, pleural fluid, pus, saliva, sebum, semen, serum, sweat, synovial fluid, tears, urine, vaginal lubrication, vitreous humor, vomit).

The samples can be collected from individuals repeatedly over a longitudinal period of time (e.g., once or more on the order of days, weeks, months, annually, biannually, etc.). Obtaining numerous samples from an individual over a period of time can be used to verify results from earlier detections and/or to identify an alteration in biological pattern as a result of, for example, disease progression, drug treatment, etc. For example, subject samples can be taken and monitored every month, every two months, or combinations of one, two, or three month intervals according to the present invention. In addition, the biomarker amount and/or activity measurements of the subject obtained over time can be conveniently compared with each other, as well as with those of normal controls during the monitoring period, thereby providing the subject's own values, as an internal, or personal, control for long-term monitoring.

Sample preparation and separation can involve any of the procedures, depending on the type of sample collected and/or analysis of biomarker measurement(s). Such procedures include, by way of example only, concentration, dilution, adjustment of pH, removal of high abundance polypeptides (e.g., albumin, gamma globulin, and transferrin, etc.), addition of preservatives and calibrants, addition of protease inhibitors, addition of denaturants, desalting of samples, concentration of sample proteins, extraction and purification of lipids.

The sample preparation can also isolate molecules that are bound in non-covalent complexes to other protein (e.g., carrier proteins). This process may isolate those molecules bound to a specific carrier protein (e.g., albumin), or use a more general process, such as the release of bound molecules from all carrier proteins via protein denaturation, for example using an acid, followed by removal of the carrier proteins.

In some embodiments, the sample can be an untreated sample. An untreated sample refers to a test sample that has not had any prior sample pre-treatment except for dilution and/or suspension in a solution.

Removal of undesired proteins (e.g., high abundance, uninformative, or undetectable proteins) from a sample can be achieved using high affinity reagents, high molecular weight filters, ultracentrifugation and/or electrodialysis. High affinity reagents include antibodies or other reagents (e.g., aptamers) that selectively bind to high abundance proteins. Sample preparation could also include ion exchange chromatography, metal ion affinity chromatography, gel filtration, hydrophobic chromatography, chromatofocusing, adsorption chromatography, isoelectric focusing and related techniques. Molecular weight filters include membranes that separate molecules on the basis of size and molecular weight. Such filters may further employ reverse osmosis, nanofiltration, ultrafiltration and microfiltration. Ultracentrifugation is a method for removing undesired polypeptides from a sample. Ultracentrifugation is the centrifugation of a sample at about 15,000-60,000 rpm while monitoring with an optical system the sedimentation (or lack thereof) of particles.

Electrodialysis is a procedure which uses an electromembrane or semipermable membrane in a process in which ions are transported through semi-permeable membranes from one solution to another under the influence of a potential gradient. Since the membranes used in electrodialysis may have the ability to selectively transport ions having positive or negative charge, reject ions of the opposite charge, or to allow species to migrate through a semipermable membrane based on size and charge, it renders electrodialysis useful for concentration, removal, or separation of electrolytes.

Separation and purification in the present invention may include any procedure known in the art, such as capillary electrophoresis (e.g., in capillary or on-chip) or chromatography (e.g., in capillary, column or on a chip). Electrophoresis is a method that can be used to separate ionic molecules under the influence of an electric field. Electrophoresis can be conducted in a gel, capillary, or in a microchannel on a chip. Examples of gels used for electrophoresis include starch, acrylamide, polyethylene oxides, agarose, or combinations thereof. A gel can be modified by its cross-linking, addition of detergents, or denaturants, immobilization of enzymes or antibodies (affinity electrophoresis) or substrates (zymography) and incorporation of a pH gradient. Examples of capillaries used for electrophoresis include capillaries that interface with an electrospray.

Capillary electrophoresis (CE) is preferred for separating complex hydrophilic molecules and highly charged solutes. CE technology can also be implemented on microfluidic chips. Depending on the types of capillary and buffers used, CE can be further segmented into separation techniques such as capillary zone electrophoresis (CZE), capillary isoelectric focusing (CIEF), capillary isotachophoresis (cITP) and capillary electrochromatography (CEC). An embodiment to couple CE techniques to electrospray ionization involves the use of volatile solutions, for example, aqueous mixtures containing a volatile acid and/or base and an organic such as an alcohol or acetonitrile.

Capillary isotachophoresis (cITP) is a technique in which the analytes move through the capillary at a constant speed but are nevertheless separated by their respective mobilities. Capillary zone electrophoresis (CZE), also known as free-solution CE (FSCE), is based on differences in the electrophoretic mobility of the species, determined by the charge on the molecule, and the frictional resistance the molecule encounters during migration which is often directly proportional to the size of the molecule. Capillary isoelectric focusing (CIEF) allows weakly-ionizable amphoteric molecules, to be separated by electrophoresis in a pH gradient. CEC is a hybrid technique between traditional high performance liquid chromatography (UPLC) and CE.

Separation and purification techniques used in the present invention include any chromatography procedures known in the art. Chromatography can be based on the differential adsorption and elution of certain analytes or partitioning of analytes between mobile and stationary phases. Different examples of chromatography include, but not limited to, liquid chromatography (LC), gas chromatography (GC), high performance liquid chromatography (UPLC), etc.

### Analyzing Biomarker Nucleic Acids and Polypeptides

Biomarker nucleic acids and/or biomarker polypeptides can be analyzed according to the methods described herein and techniques known to the skilled artisan to identify such genetic or expression alterations useful for the present invention including, but not limited to, an alteration in the level of a biomarker transcript or polypeptide, and the like.

In some embodiments, the expression level of a biomarker disclosed can be measured by determining the level of an expression product of the biomarker, e.g., a RNA transcript or a polypeptide. Such molecules can be isolated, derived, or amplified from a biological sample, such as a biofluid or biological sample such as a cervical swab.

a. Methods for Detection of Biomarker Nucleic Acid Expression

Biomarker expression may be assessed by any of a wide variety of well-known methods for detecting expression of a transcribed molecule or protein. Non-limiting examples of such methods include immunological methods for detection of secreted, cell-surface, cytoplasmic, or nuclear proteins, protein purification methods, protein function or activity assays, nucleic acid hybridization methods, nucleic acid reverse transcription methods, and nucleic acid amplification methods.

In preferred embodiments, activity of a particular gene is characterized by a measure of gene transcript (e.g. mRNA), by a measure of the quantity of translated protein, or by a measure of gene product activity. Marker expression can be monitored in a variety of ways, including by detecting mRNA levels, protein levels, or protein activity, any of which can be measured using standard techniques. Detection can involve quantification of the level of gene expression (e.g., genomic DNA, cDNA, mRNA, protein, or enzyme activity), or, alternatively, can be a qualitative assessment of the level of gene expression, in particular in comparison with a control level. The type of level being detected will be clear from the context.

Assays for detecting mRNA transcripts are well known in the art and include, but are not limited to, PCR procedures, RT-PCR, Northern blot analysis, RNAse protection assay, microarray analysis, hybridization methods etc. In some embodiments, mRNA transcript expression product levels are assayed using reverse transcription polymerase chain reaction (RT-PCR).

In another embodiment, detecting or determining expression levels of a biomarker and functionally similar homologs thereof, including a fragment or genetic alteration thereof (e.g., in regulatory or promoter regions thereof) comprises detecting or determining RNA levels for the marker of interest. In one embodiment, one or more cells from the subject to be tested are obtained and RNA is isolated from the cells.

In one embodiment, RNA is obtained from a single cell. For example, a cell can be isolated from a tissue sample by laser capture microdissection (LCM). Using this technique, a cell can be isolated from a tissue section, including a stained tissue section, thereby assuring that the desired cell is isolated (see, e.g., Bonner et al. (1997) Science 278: 1481; Emmert-Buck et al. (1996) Science 274:998; Fend et al. (1999) Am. J. Path. 154: 61 and Murakami et al. (2000) Kidney Int. 58:1346). For example, Murakami et al., supra, describe isolation of a cell from a previously immunostained tissue section.

It is also be possible to obtain cells from a subject and culture the cells in vitro, such as to obtain a larger population of cells from which RNA can be extracted. Methods for establishing cultures of non-transformed cells, i.e., primary cell cultures, are known in the art.

When isolating RNA from tissue samples or cells from individuals, it may be important to prevent any further changes in gene expression after the tissue or cells has been removed from the subject. Changes in expression levels are known to change rapidly following perturbations, e.g., heat shock or activation with lipopolysaccharide (LPS) or other reagents. In addition, the RNA in the tissue and cells may quickly become degraded. Accordingly, in a preferred embodiment, the tissue or cells obtained from a subject is snap frozen as soon as possible.

RNA can be extracted from the tissue sample by a variety of methods, e.g., the guanidium thiocyanate lysis followed by CsCl centrifugation (Chirgwin et al., 1979, Biochemistry 18:5294-5299). RNA from single cells can be obtained as described in methods for preparing cDNA libraries from single cells, such as those described in Dulac, C. (1998) Curr. Top. Dev. Biol. 36, 245 and Jena et al. (1996) J. Immunol. Methods 190:199. Care to avoid RNA degradation must be taken, e.g., by inclusion of RNAsin.

The RNA sample can then be enriched in particular species. In one embodiment, poly(A)+RNA is isolated from the RNA sample. In general, such purification takes advantage of the poly-A tails on mRNA. In particular and as noted above, poly-T oligonucleotides may be immobilized within on a solid support to serve as affinity ligands for mRNA. Kits for this purpose are commercially available, e.g., the MessageMaker kit (Life Technologies, Grand Island, N.Y.).

In an embodiment, the RNA population is enriched in marker sequences. Enrichment can be undertaken, e.g., by primer-specific cDNA synthesis, or multiple rounds of linear amplification based on cDNA synthesis and template-directed in vitro transcription (see, e.g., Wang et al. (1989) PNAS 86, 9717; Dulac et al., supra, and Jena et al., supra).

The population of RNA, enriched or not in particular species or sequences, can further be amplified. As defined herein, an “amplification process” is designed to strengthen, increase, or augment a molecule within the RNA. For example, where RNA is mRNA, an amplification process such as RT-PCR can be utilized to amplify the mRNA, such that a signal is detectable or detection is enhanced. Such an amplification process is beneficial particularly when the biological, tissue, or tumor sample is of a small size or volume.

Various amplification and detection methods can be used. For example, it is within the scope of the present invention to reverse transcribe mRNA into cDNA followed by polymerase chain reaction (RT-PCR); or, to use a single enzyme for both steps as described in U.S. Pat. No. 5,322,770, or reverse transcribe mRNA into cDNA followed by symmetric gap ligase chain reaction (RT-AGLCR) as described by R. L. Marshall, et al., PCR Methods and Applications 4: 80-84 (1994). Real time PCR may also be used. In general, the PCR procedure describes a method of gene amplification which is comprised of (i) sequence-specific hybridization of primers to specific genes within a nucleic acid sample or library, (ii) subsequent amplification involving multiple rounds of annealing, elongation, and denaturation using a thermostable DNA polymerase, and (iii) screening the PCR products for a band of the correct size or for hybridization to a given probe. The primers used are oligonucleotides of sufficient length and appropriate sequence to provide initiation of polymerization, i.e. each primer is specifically designed to be complementary to a strand of the genomic locus to be amplified. In an alternative embodiment, mRNA level of gene expression products described herein can be determined by reverse-transcription (RT) PCR and by quantitative RT-PCR (QRT-PCR) or real-time PCR methods. Methods of RT-PCR and QRT-PCR are well known in the art.

Other known amplification methods which can be utilized herein include but are not limited to the so-called “NASBA” or “3SR” technique described in PNAS USA 87: 1874-1878 (1990) and also described in Nature 350 (No. 6313): 91-92 (1991); Q-beta amplification as described in published European Patent Application (EPA) No. 4544610; strand displacement amplification (as described in G. T. Walker et al., Clin. Chem. 42: 9-13 (1996) and European Patent Application No. 684315; target mediated amplification, as described by PCT Publication WO9322461; PCR; ligase chain reaction (LCR) (see, e.g., Wu and Wallace, Genomics 4, 560 (1989), Landegren et al., Science 241, 1077 (1988)); self-sustained sequence replication (SSR) (see, e.g., Guatelli et al., Proc. Nat. Acad. Sci. USA, 87, 1874 (1990)); and transcription amplification (see, e.g., Kwoh et al., Proc. Natl. Acad. Sci. USA 86, 1173 (1989)).

Many techniques are known in the state of the art for determining absolute and relative levels of gene expression, commonly used techniques suitable for use in the present invention include Northern analysis, RNase protection assays (RPA), microarrays and PCR-based techniques, such as quantitative PCR and differential display PCR. For example, Northern blotting involves running a preparation of RNA on a denaturing agarose gel, and transferring it to a suitable support, such as activated cellulose, nitrocellulose or glass or nylon membranes. Radiolabeled cDNA or RNA is then hybridized to the preparation, washed and analyzed by autoradiography.

In situ hybridization visualization may also be employed, wherein a radioactively labeled antisense RNA probe is hybridized with a thin section of a biopsy sample, washed, cleaved with RNase and exposed to a sensitive emulsion for autoradiography. The samples may be stained with hematoxylin to demonstrate the histological composition of the sample, and dark field imaging with a suitable light filter shows the developed emulsion. Non-radioactive labels such as digoxigenin may also be used.

Alternatively, mRNA expression can be detected on a DNA array, chip or a microarray. Labeled nucleic acids of a test sample obtained from a subject may be hybridized to a solid surface comprising biomarker DNA. Positive hybridization signal is obtained with the sample containing biomarker transcripts. Methods of preparing DNA arrays and their use are well known in the art (see, e.g., U.S. Pat. Nos: 6,618,6796; 6,379,897; 6,664,377; 6,451,536; 548,257; U.S. 20030157485 and Schena et al. (1995) Science 20, 467-470; Gerhold et al. (1999) Trends In Biochem. Sci. 24, 168-173; and Lennon et al. (2000) Drug Discovery Today 5, 59-65, which are herein incorporated by reference in their entirety). Serial Analysis of Gene Expression (SAGE) can also be performed (See for example U.S. Patent Application 20030215858).

To monitor mRNA levels, for example, mRNA is extracted from the biological sample to be tested, reverse transcribed, and fluorescently-labeled cDNA probes are generated. The microarrays capable of hybridizing to marker cDNA are then probed with the labeled cDNA probes, the slides scanned and fluorescence intensity measured. This intensity correlates with the hybridization intensity and expression levels.

Types of probes that can be used in the methods described herein include cDNA, riboprobes, synthetic oligonucleotides and genomic probes. The type of probe used will generally be dictated by the particular situation, such as riboprobes for in situ hybridization, and cDNA for Northern blotting, for example. In one embodiment, the probe is directed to nucleotide regions unique to the RNA. The probes may be as short as is required to differentially recognize marker mRNA transcripts, and may be as short as, for example, 15 bases; however, probes of at least 17, 18, 19 or 20 or more bases can be used. In one embodiment, the primers and probes hybridize specifically under stringent conditions to a DNA fragment having the nucleotide sequence corresponding to the marker. As herein used, the term “stringent conditions” means hybridization will occur only if there is at least 95% identity in nucleotide sequences. In another embodiment, hybridization under “stringent conditions” occurs when there is at least 97% identity between the sequences.

The form of labeling of the probes may be any that is appropriate, such as the use of radioisotopes, for example, ³²P and ³⁵. Labeling with radioisotopes may be achieved, whether the probe is synthesized chemically or biologically, by the use of suitably labeled bases.

In one embodiment, the biological sample contains polypeptide molecules from the test subject. Alternatively, the biological sample can contain mRNA molecules from the test subject or genomic DNA molecules from the test subject.

In another embodiment, the methods further involve obtaining a control biological sample from a control subject, contacting the control sample with a compound or agent capable of detecting marker polypeptide, mRNA, genomic DNA, or fragments thereof, such that the presence of the marker polypeptide, mRNA, genomic DNA, or fragments thereof, is detected in the biological sample, and comparing the presence of the marker polypeptide, mRNA, genomic DNA, or fragments thereof, in the control sample with the presence of the marker polypeptide, mRNA, genomic DNA, or fragments thereof in the test sample.

b. Methods for Detection of Biomarker Protein Expression

The activity or level of a biomarker protein can be detected and/or quantified by detecting or quantifying the expressed polypeptide. The polypeptide can be detected and quantified by any of a number of means well known to those of skill in the art. Any method known in the art for detecting polypeptides can be used. Such methods include, but are not limited to, immunodiffusion, immunoelectrophoresis, radioimmunoassay (RIA), enzyme-linked immunosorbent assays (ELISAs), immunofluorescent assays, Western blotting, binder-ligand assays, immunohistochemical techniques, agglutination, complement assays, high performance liquid chromatography (HPLC), thin layer chromatography (TLC), hyperdiffusion chromatography, and the like (e.g., Basic and Clinical Immunology, Sites and Terr, eds., Appleton and Lange, Norwalk, Conn. pp 217-262, 1991 which is incorporated by reference). Preferred are binder-ligand immunoassay methods including reacting antibodies with an epitope or epitopes and competitively displacing a labeled polypeptide or derivative thereof.

Detection of polypeptides encoded by the biomarkers disclosed herein can be according to any method known in the art. Immunological methods to detect the polypeptides in accordance with the present technology include, but are not limited to antibody techniques such as immunohistochemistry, immunocytochemistry, flow cytometry, fluorescent-activated cell sorting (FACS), immunoblotting, radioimmunoassays, western blotting, immunoprecipitation, enzyme-linked immunosorbant assays (ELISA), and derivative techniques that make use of antibody reagents as described herein.

Immunochemical methods require the use of an antibody reagent specific for the target molecule (e.g. the antigen or in the embodiments described herein, a polypeptide or fragment thereof). In some embodiments, an antibody reagent for measuring the level of a polypeptide in a sample can be an antibody reagent specific for a polypeptide encoded by a biomarker gene disclosed herein.

For example, ELISA and RIA procedures may be conducted such that a desired biomarker protein standard is labeled (with a radioisotope such as ¹²⁵I or ³⁵S, or an assayable enzyme, such as horseradish peroxidase or alkaline phosphatase), and, together with the unlabeled sample, brought into contact with the corresponding antibody, whereon a second antibody is used to bind the first, and radioactivity or the immobilized enzyme assayed (competitive assay). Alternatively, the biomarker protein in the sample is allowed to react with the corresponding immobilized antibody, radioisotope- or enzyme-labeled anti-biomarker protein antibody is allowed to react with the system, and radioactivity or the enzyme assayed (ELISA-sandwich assay). Other conventional methods may also be employed as suitable.

The above techniques may be conducted essentially as a “one-step” or “two-step” assay. A “one-step” assay involves contacting antigen with immobilized antibody and, without washing, contacting the mixture with labeled antibody. A “two-step” assay involves washing before contacting, the mixture with labeled antibody. Other conventional methods may also be employed as suitable.

In one embodiment, a method for measuring biomarker protein levels comprises the steps of: contacting a biological specimen with an antibody or variant (e.g., fragment) thereof which selectively binds the biomarker protein, and detecting whether said antibody or variant thereof is bound to said sample and thereby measuring the levels of the biomarker protein.

Enzymatic and radiolabeling of biomarker protein and/or the antibodies may be effected by conventional means. Such means will generally include covalent linking of the enzyme to the antigen or the antibody in question, such as by glutaraldehyde, specifically so as not to adversely affect the activity of the enzyme, by which is meant that the enzyme must still be capable of interacting with its substrate, although it is not necessary for all of the enzyme to be active, provided that enough remains active to permit the assay to be effected. Indeed, some techniques for binding enzyme are non-specific (such as using formaldehyde), and will only yield a proportion of active enzyme.

It is usually desirable to immobilize one component of the assay system on a support, thereby allowing other components of the system to be brought into contact with the component and readily removed without laborious and time-consuming labor. It is possible for a second phase to be immobilized away from the first, but one phase is usually sufficient.

It is possible to immobilize the enzyme itself on a support, but if solid-phase enzyme is required, then this is generally best achieved by binding to antibody and affixing the antibody to a support, models and systems for which are well known in the art. Simple polyethylene may provide a suitable support.

Enzymes employable for labeling are not particularly limited, but may be selected from the members of the oxidase group, for example. These catalyze production of hydrogen peroxide by reaction with their substrates, and glucose oxidase is often used for its good stability, ease of availability and cheapness, as well as the ready availability of its substrate (glucose). Activity of the oxidase may be assayed by measuring the concentration of hydrogen peroxide formed after reaction of the enzyme-labeled antibody with the substrate under controlled conditions well-known in the art.

Other techniques may be used to detect biomarker protein according to a practitioner's preference based upon the present disclosure. One such technique is Western blotting (Towbin et at., Proc. Nat. Acad. Sci. 76:4350 (1979)), wherein a suitably treated sample is run on an SDS-PAGE gel before being transferred to a solid support, such as a nitrocellulose filter. Anti-biomarker protein antibodies (unlabeled) are then brought into contact with the support and assayed by a secondary immunological reagent, such as labeled protein A or anti-immunoglobulin (suitable labels including ¹²⁵I, horseradish peroxidase and alkaline phosphatase). Chromatographic detection may also be used.

Immunohistochemistry may be used to detect expression of biomarker protein, e.g., in a biopsy sample. A suitable antibody is brought into contact with, for example, a thin layer of cells, washed, and then contacted with a second, labeled antibody. Labeling may be by fluorescent markers, enzymes, such as peroxidase, avidin, or radiolabelling. The assay is scored visually, using microscopy.

Anti-biomarker protein antibodies, such as intrabodies, may also be used for imaging purposes, for example, to detect the presence of biomarker protein in cells and tissues of a subject. Suitable labels include radioisotopes, iodine (¹²⁵, ¹²¹I), carbon (¹⁴C), sulphur (³⁵S), tritium (³H), indium (¹¹²In), and technetium (⁹⁹mTc), fluorescent labels, such as fluorescein and rhodamine, and biotin.

In other embodiments, the detection antibody is labeled with a fluorescent compound. When the fluorescently labeled antibody is exposed to light of the proper wavelength, its presence can then be detected due to fluorescence. In some embodiments, a detectable label can be a fluorescent dye molecule, or fluorophore including, but not limited to fluorescein, phycoerythrin, phycocyanin, o-phthaldehyde, fluorescamine, Cy3™, Cy5™, allophycocyanine, Texas Red, pefidenin chlorophyll, cyanine, tandem conjugates such as phycoerythrin-Cy5™, green fluorescent protein, rhodamine, fluorescein isothiocyanate (FITC) and Oregon Green™, rhodamine and derivatives (e.g., Texas red and tetrarhodimine isothiocynate (TRITC)), biotin, phycoerythrin, AMCA, CyDyes™, 6-carboxyfluorescein (commonly known by the abbreviations FAM and F), 6-carboxy-2′,4′,7′,4,7-hexachlorofiuorescein (HEX), 6-carboxy-4′,5′-dichloro-2′,7′-dimethoxyfluroescein (JOE or N,N,N′,N′-tetramethyl-6carboxyrhodamine (TAMRA, or T), 6-carboxy-X-rhodamine (ROX or R), 5-carboxyrhodamine-6G (R6G5 or G5), 6-carboxyrhodamine-60 (R6G6 or G6), and rhodamine 110; cyanine dyes, e.g. Cy3, Cy5 and Cy7 dyes; coumarins, e.g umbelliferone; benzimide dyes, e.g. Hoechst 33258; phenanthridine dyes, e.g. Texas Red; ethidium dyes; acridine dyes; carbazole dyes; phenoxazine dyes; porphyrin dyes; polymethine dyes, e.g. cyanine dyes such as Cy3, Cy5, etc; BODIPY dyes and quinoline dyes.

For in vivo imaging purposes, antibodies are not detectable, as such, from outside the body, and so must be labeled or otherwise modified, to permit detection. Markers for this purpose may be any that do not substantially interfere with the antibody binding, but which allow external detection. Suitable markers may include those that may be detected by X-radiography, NMR or MRI. For X-radiographic techniques, suitable markers include any radioisotope that emits detectable radiation but that is not overtly harmful to the subject, such as barium or cesium, for example. Suitable markers for NMR and MRI generally include those with a detectable characteristic spin, such as deuterium, which may be incorporated into the antibody by suitable labeling of nutrients for the relevant hybridoma, for example.

The size of the subject, and the imaging system used, will determine the quantity of imaging moiety needed to produce diagnostic images. In the case of a radioisotope moiety, for a human subject, the quantity of radioactivity injected will normally range from about 5 to 20 millicuries of technetium-99. The labeled antibody or antibody fragment will then preferentially accumulate at the location of cells which contain biomarker protein. The labeled antibody or antibody fragment can then be detected using known techniques.

Antibodies that may be used to detect biomarker protein include any antibody, whether natural or synthetic, full length or a fragment thereof, monoclonal or polyclonal, that binds sufficiently strongly and specifically to the biomarker protein to be detected. An antibody may have a K_{d} of at most about 10⁻⁶M, 10⁻⁷M, 10⁻⁸ M, 10⁻⁹ M, 10⁻¹⁰M, 10⁻¹¹ M, 10⁻¹²M. The phrase “specifically binds” refers to binding of, for example, an antibody to an epitope or antigen or antigenic determinant in such a manner that binding can be displaced or competed with a second preparation of identical or similar epitope, antigen or antigenic determinant. An antibody may bind preferentially to the biomarker protein relative to other proteins, such as related proteins.

Antibodies are commercially available or may be prepared according to methods known in the art.

Antibodies and derivatives thereof that may be used encompass polyclonal or monoclonal antibodies, chimeric, human, humanized, primatized (CDR-grafted), veneered or single-chain antibodies as well as functional fragments, i.e., biomarker protein binding fragments, of antibodies. For example, antibody fragments capable of binding to a biomarker protein or portions thereof, including, but not limited to, Fv, Fab, Fab′ and F(ab′) 2 fragments can be used. Such fragments can be produced by enzymatic cleavage or by recombinant techniques. For example, papain or pepsin cleavage can generate Fab or F(ab′) 2 fragments, respectively. Other proteases with the requisite substrate specificity can also be used to generate Fab or F(ab′) 2 fragments. Antibodies can also be produced in a variety of truncated forms using antibody genes in which one or more stop codons have been introduced upstream of the natural stop site. For example, a chimeric gene encoding a F(ab′) 2 heavy chain portion can be designed to include DNA sequences encoding the CH, domain and hinge region of the heavy chain.

Synthetic and engineered antibodies are described in, e.g., Cabilly et al., U.S. Pat. No. 4,816,567 Cabilly et al., European Patent No. 0,125,023 B1; Boss et al., U.S. Pat. No. 4,816,397; Boss et al., European Patent No. 0,120,694 B1; Neuberger, M. S. et al., WO 86/01533; Neuberger, M. S. et al., European Patent No. 0,194,276 B1; Winter, U.S. Pat. No. 5,225,539; Winter, European Patent No. 0,239,400 B1; Queen et al., European Patent No. 0451216 B1; and Padlan, E. A. et al., EP 0519596 A1. See also, Newman, R. et al., BioTechnology, 10: 1455-1460 (1992), regarding primatized antibody, and Ladner et al., U.S. Pat. No. 4,946,778 and Bird, R. E. et al., Science, 242: 423-426 (1988)) regarding single-chain antibodies. Antibodies produced from a library, e.g., phage display library, may also be used.

In some embodiments, agents that specifically bind to a biomarker protein other than antibodies are used, such as peptides. Peptides that specifically bind to a biomarker protein can be identified by any means known in the art. For example, specific peptide binders of a biomarker protein can be screened for using peptide phage display libraries.

### Additional Treatment Methods

Elevated levels of a gene product of KRT1, PZP, KRT14, HP, CA1, HBB, SAA1, IGHG4, PRPS1, RBMX, NSF, IGKV2D-29, and/or PGRMC1 are associated with endometriosis-related conditions as demonstrated herein. Accordingly, provided herein are methods of treating endometriosis-related conditions. In one aspect, described herein is a method of treating an endometriosis-related condition, the method comprising administering a therapeutically effective amount of a biomarker inhibitor to a subject in need of treatment (e.g., an inhibitor of a gene product of a biomarker disclosed herein).

As used herein, the term “inhibitor of a biomaker” refers to an agent that can decrease the expression level and/or activity of a biomarker disclosed herein, e.g. by at least 1%, at least 5%, at least 10%, at least 20%, at least 30%, at least 40%, at least 50%, at least 75%, at least 80%, at least 90%, at least 95%, at least 98%, at least 99% or more. In some embodiments, an inhibitor can decrease the level of mRNA or the level of a biomarker polypeptide. In some embodiments, an inhibitor can specifically bind an expression product of the biomarker. In some embodiments, a biomarker inhibitor can specifically bind a polypeptide.

In some aspects, provided herein are methods of treating or preventing an endometriosis related condition by administering a therapeutically effective amount of an inhibitor of at least one biomarker (e.g., a biomarker disclosed herein, such as the expression of, activity of, or levels of a gene product of KRT1, PZP, KRT14, HP, CA1, HBB, SAA1, IGHG4, PRPS1, RBMX, NSF, IGKV2D-29, and/or PGRMC1) to a subject in need of treatment. In one aspect, described herein is a method of treating an endometriosis-related condition, the method comprising; administering a therapeutically effective amount of a biomarker (e.g., a biomarker disclosed herein) binding reagent associated with a therapeutic agent to a subject in need of treatment. In some embodiments, the therapeutic agent can be a toxic moiety. In some embodiments, the endometriosis-related condition can be selected from the group consisting of: endometriosis; endometriosis cysts; endometrioid cancer, infertility/subfertility, ovarian cyst, uterine fibroids, pelvic inflammatory disorder or ovarian cancer. In some embodiments, the biomarker inhibitor can specifically bind to a protein or mRNA produce of at least one biomarker (e.g., a biomarker disclosed herein, such as KRT1, PZP, KRT14, HP, CA1, HBB, SAA1, IGHG4, PRPS1, RBMX, NSF, IGKV2D-29, and/or PGRMC1) polypeptide.

In one aspect, described herein is a method of administering a treatment for endometriosis or an endometriosis-related condition to a subject, the method comprising: determining the expression level of, activity of, or levels of a product of at least one biomarker (e.g., a biomarker disclosed herein, such as KRT1, PZP, KRT14, HP, CA1, HBB, SAA1, IGHG4, PRPS1, RBMX, NSF, IGKV2D-29, and/or PGRMC1) polypeptide in a test sample obtained from a subject; and administering a treatment for endometriosis or endometriosis-related condition to the subject if the expression level of the at least one biomarker is increased relative to a reference level. As used herein, a “reference level” or “control level” may be the RNA level, protein level, or protein activity level of the biomarker in a subject who is not afflicted with endometriosis or an endometriosis-related condition.

In one aspect, described herein is a method of administering a treatment for endometriosis or an endometriosis-related condition to a subject, the method comprising administering a treatment for endometriosis or the endometriosis-related condition to a subject determined to have an increased the expression level of, activity of, or levels of a product of at least one biomarker (e.g., a biomarker disclosed herein, such as KRT1, PZP, KRT14, HP, CA, HBB, SAA1, IGHG4, PRPS1, RBMX, NSF, IGKV2D-29, and/or PGRMC1) in a test sample obtained from the subject; wherein the expression level of the at least one is an increased level if it is increased relative to a reference level.

In one aspect, described herein is a method of identifying a subject in need of treatment for endometriosis or endometriosis-related condition, the method comprising: determining the expression level of or activity of a protein or RNA product of at least one biomarker (e.g., a biomarker disclosed herein, such as KRT1, PZP, KRT14, HP, CA1, HBB, SAA1, IGHG4, PRPS1, RBMX, NSF, IGKV2D-29, and/or PGRMC1) in a test sample obtained from a subject; wherein the subject is identified as being in need of treatment for endometriosis if the expression level of the biomarker is increased relative to a reference level.

In one aspect, described herein is a method of identifying a subject in need of a laparoscopic examination, the method comprising: determining the expression level or activity of a protein or the levels of an mRNA product of at least one biomarker (e.g., a biomarker disclosed herein, such as KRT1, PZP, KRT14, HP, CA1, HBB, SAA1, IGHG4, PRPS1, RBMX, NSF, IGKV2D-29, and/or PGRMC) in a test sample obtained from a subject; wherein the subject is identified as being in need of a laparoscopic examination if the expression level of the biomarker is increased relative to a reference level.

In one aspect, described herein is a method of determining the efficacy of a treatment for endometriosis or an endometriosis-related condition, the method comprising: (a) determining the expression level of, activity of, or levels of a product of at least one biomarker (e.g., a biomarker disclosed herein, such as KRT1, PZP, KRT14, HP, CA1, HBB, SAA1, IGHG4, PRPS1, RBMX, NSF, IGKV2D-29, and/or PGRMC1) in a test sample obtained from a subject before administration of the treatment; (b) determining the expression level of the biomarker in a test sample obtained from a subject after administration of the treatment; wherein the treatment is not efficacious if the expression level determined in step (b) is increased relative to the expression level determined in step (a).

In some embodiments, the treatment for endometriosis or endometriosis-related condition can be selected from the group consisting of: a hormonal treatment; progesterone; progestin; an oral contraceptive; a hormonal contraceptive; danocrine; gentrinone; a gonadotrophin releasing hormone agonist; Lupron; danazol; an aromatase inhibitor; pentoxifylline; surgical treatment; laparoscopy; cauterization; and cystectomy. In some embodiments, the sample is a biological sample and can comprise a material selected from the group consisting of: a biofluid sample, serum, plasma, urine, saliva, yolk sac, an endometrial tissue sample; a tumor sample, a cyst, an ovarian cyst, cystic fluid, peritoneal fluid, pleural fluid, and a cervical swab.

The term “sample” or “test sample” as used herein denotes a sample taken or isolated from an organism, e.g., a cervical sample from a subject. Exemplary biological samples include, but are not limited to, a biofluid sample: serum; plasma; urine; saliva; yolk sac; an endometrial tissue sample; a tumor sample; a cyst; an ovarian cyst; cystic fluid; peritoneal fluid; pleural fluid; and/or a cervical swab; etc. The term also includes a mixture of the above-mentioned samples. The term “test sample” also includes untreated or pretreated (or pre-processed) biological samples. In some embodiments, a test sample can comprise cells from a subject.

In some embodiments, the sample can comprise any tissue affected by symptoms or, or displaying markers of endometriosis, e.g. the sample can comprise cysts. In some embodiments, the test sample can comprise or consist of urine. In some embodiments, the test sample can comprise or consist of blood and/or blood products, e.g. serum and/or plasma. As used herein, the term “biofluid” refers to any fluid obtained from a biological source and includes, but is not limited to, blood, urine, cystic fluids, and bodily secretions.

In some embodiments, endometriosis has or is at risk of progressing to another endometriosis-related condition, such as endometriosis cyst; ovarian carcinoma; and clear cell ovarian cancer. In some embodiments, the expression level of the at least one biomarker (e.g., the expression level of a gene disclosed herein or the amount or activity of a protein disclosed herein) can be normalized relative to the expression level of one or more reference products of genes or reference proteins. In some embodiments, the reference expression level of the at least one biomarker can be the level of the at least one biomarker in a prior sample obtained from the subject.

Preferably, the subject is a mammal. The mammal can be a human, non-human primate, mouse, rat, dog, cat, horse, or cow, but is not limited to these examples. Mammals other than humans can be advantageously used as subjects that represent animal models of, e.g. endometriosis or an endometriosis-related condition. A subject can be male or female. In embodiments relating to endometriosis and/or an endometriosis-related condition, a subject can be female.

A subject can be one who has been previously diagnosed with or identified as suffering from or having a condition in need of treatment (e.g. endometriosis or ovarian cancer) or one or more complications related to such a condition, and optionally, have already undergone treatment for, e.g., endometriosis or the one or more complications related to endometriosis.

Symptoms of endometriosis can include, but are not limited to, pelvic pain, infertility, and endometrial adhesions, and endometrial hemorrhagic or fibrotic foci. Alternatively, a subject can also be one who has not been previously diagnosed as having, e.g., endometriosis or one or more complications related to endometriosis. For example, a subject can be one who exhibits one or more risk factors for, e.g., endometriosis or one or more complications related to endometriosis or a subject who does not exhibit risk factors.

As used herein, the term “agent” refers to any agent that can have a therapeutic effect and/or treat an endometriosis-related condition, e.g. can decrease the severity of a sign, symptom, and/or marker of an endometriosis-related condition. The therapeutic methods described herein can be used to treat any endometriosis-related condition, including but not limited to endometriosis; endometriosis cysts; endometrioid cancer; ovarian cancer; and clear cell cancer. An agent may treat or prevent an endometriosis-related condition by inhibiting the activity of or decreasing the levels of a product of a biomarker disclosed herein.

a. Polypeptide Agents

In certain embodiments, a polypeptide agent is used as an inhibitor in the methods and compositions disclosed herein. In some embodiments, the polypeptide agent is an isolated polypeptide that specifically binds to and inhibits the activity of a protein product of a biomarker disclosed herein.

In some embodiments, the polypeptide agents disclosed herein can be isolated from cells or tissue sources by an appropriate purification scheme using standard protein purification techniques. In another embodiment, polypeptide agents are produced by recombinant DNA techniques. Alternatively, polypeptides disclosed herein can be chemically synthesized using standard peptide synthesis techniques.

In some embodiments, the polypeptide agent is a chimeric or fusion proteins. As used herein, a “chimeric protein” or “fusion protein” comprises a polypeptide or protein described herein (e.g., a protein that specifically binds to a protein product of a biomarker disclosed herein) linked to a distinct polypeptide to which it is not linked in nature. For example, the distinct polypeptide can be fused to the N-terminus or C-terminus of the polypeptide either directly, through a peptide bond, or indirectly through a chemical linker. In some embodiments, the peptide described herein is linked to an immunoglobulin constant domain (e.g., an IgG constant domain, such as a human IgG constant domain).

The polypeptide agents provided herein can be generated according to any method available in the art. For example, the polypeptide agents can be produced in prokaryotic or eukaryotic host cells by expression of polynucleotides encoding a polypeptide(s) described herein. Alternatively, such peptides can be synthesized by chemical methods. Methods for expression of heterologous polypeptides in recombinant hosts, chemical synthesis of polypeptides, and in vitro translation are well known in the art and are described further in Maniatis et al., Molecular Cloning: A Laboratory Manual (1989), 2nd Ed., Cold Spring Harbor, N.Y.; Berger and Kimmel, Methods in Enzymology, Volume 152, Guide to Molecular Cloning Techniques (1987), Academic Press, Inc., San Diego, Calif.; Merrifield, J. (1969) J. Am. Chem. Soc. 91:501; Chaiken I. M. (1981) CRC Crit. Rev. Biochem. 11:255; Kaiser et al. (1989) Science 243:187; Merrifield, B. (1986) Science 232:342; Kent, S. B. H. (1988) Annu. Rev. Biochem. 57:957; and Offord, R. E. (1980) Semisynthetic Proteins, Wiley Publishing, which are incorporated herein by reference.

b. Antibody Agents

In certain embodiments, the methods and compositions provided herein relate to antibodies and antigen binding fragments thereof that bind specifically to protein product of a biomarker described herein. In some embodiments, the antibodies inhibit the activity of a protein product of a biomarker disclosed herein. In some embodiments, the antibodies inhibit the interaction between a receptor provided herein and its corresponding ligand. Such antibodies can be polyclonal or monoclonal and can be, for example, murine, chimeric, humanized or fully human.

Polyclonal antibodies can be prepared by immunizing a suitable subject (e.g. a mouse) with a polypeptide antigen. The polypeptide antibody titer in the immunized subject can be monitored over time by standard techniques, such as with an enzyme linked immunosorbent assay (ELISA) using immobilized polypeptide. If desired, the antibody directed against the antigen can be isolated from the mammal (e.g., from the blood) and further purified by well-known techniques, such as protein A chromatography to obtain the IgG fraction.

At an appropriate time after immunization, e.g., when the antibody titers are highest, antibody-producing cells can be obtained from the subject and used to prepare monoclonal antibodies using standard techniques, such as the hybridoma technique originally described by Kohler and Milstein (1975) Nature 256:495-497) (see also Brown et al. (1981) J. Immunol. 127:539-46; Brown et al. (1980) J. Biol. Chem. 255:4980-83; Yeh et al. (1976) Proc. Natl. Acad. Sci. 76:2927-31; and Yeh et al. (1982) Int. J. Cancer 29:269-75), the more recent human B cell hybridoma technique (Kozbor et al. (1983) Immunol. Today 4:72), the EBV-hybridoma technique (Cole et al. (1985) Monoclonal Antibodies and Cancer Therapy, Alan R. Liss, Inc., pp. 77-96) or trioma techniques. The technology for producing monoclonal antibody hybridomas is well known (see generally Kenneth, R. H. in Monoclonal Antibodies: A New Dimension In Biological Analyses, Plenum Publishing Corp., New York, N.Y. (1980); Lerner, E. A. (1981) Yale J. Biol. Med. 54:387-402; Gefter, M. L. et al. (1977) Somatic Cell Genet. 3:231-36). Briefly, an immortal cell line (typically a myeloma) is fused to lymphocytes (typically splenocytes) from a mammal immunized with an immunogen as described above, and the culture supernatants of the resulting hybridoma cells are screened to identify a hybridoma producing a monoclonal antibody that binds to the polypeptide antigen, preferably specifically.

As an alternative to preparing monoclonal antibody-secreting hybridomas, a monoclonal specific for a receptor or ligand provided herein can be identified and isolated by screening a recombinant combinatorial immunoglobulin library (e.g., an antibody phage display library or an antibody yeast display library) with the appropriate polypeptide to thereby isolate immunoglobulin library members that bind the polypeptide.

Additionally, recombinant antibodies specific for a receptor or ligand provided herein, such as chimeric or humanized monoclonal antibodies, can be made using standard recombinant DNA techniques. Such chimeric and humanized monoclonal antibodies can be produced by recombinant DNA techniques known in the art, for example using methods described in U.S. Pat. Nos. 4,816,567; 5,565,332; Better et al. (1988) Science 240:1041-1043; Liu et al. (1987) Proc. Nat. Acad. Sci. USA 84:3439-3443; Liu et al. (1987) J. Immunol. 139:3521-3526; Sun et al. (1987) Proc. Nat. Acad. Sci. 84:214-218; Nishimura et al. (1987) Cancer Res. 47:999-1005; Wood et al. (1985) Nature 314:446-449; and Shaw et al. (1988) J. Nat. Cancer Inst. 80:1553-1559); Morrison, S. L. (1985) Science 229:1202-1207; Oi et al. (1986) Biotechniques 4:214; Winter U.S. Pat. No. 5,225,539; Jones et al. (1986) Nature 321:552-525; Verhoeyan et al. (1988) Science 239:1534; and Beidler et al. (1988) J. Immunol. 141:4053-4060.

Human monoclonal antibodies specific for a receptor or ligand provided herein can be generated using transgenic or transchromosomal mice carrying parts of the human immune system rather than the mouse system. For example, “HuMAb mice” which contain a human immunoglobulin gene miniloci that encodes unrearanged human heavy (p and 7) and x light chain immunoglobulin sequences, together with targeted mutations that inactivate the endogenous μ and κ chain loci (Lonberg, N. et al. (1994) Nature 368(6474): 856 859). Accordingly, the mice exhibit reduced expression of mouse IgM or κ, and in response to immunization, the introduced human heavy and light chain transgenes undergo class switching and somatic mutation to generate high affinity human IgGκ monoclonal antibodies (Lonberg, N. et al. (1994), supra; reviewed in Lonberg, N. (1994) Handbook of Experimental Pharmacology 113:49 101; Lonberg, N. and Huszar, D. (1995) Intern. Rev. Immunol. Vol. 13: 65 93, and Harding, F. and Lonberg, N. (1995) Ann. N. Y Acad. Sci 764:536 546). The preparation of HuMAb mice is described in Taylor, L. et al. (1992) Nucleic Acids Research 20:6287 6295; Chen, J. et al. (1993) International Immunology 5: 647 656; Tuaillon et al. (1993) Proc. Natl. Acad. Sci USA 90:3720 3724; Choi et al. (1993) Nature Genetics 4:117 123; Chen, J. et al. (1993) EMBO J. 12: 821830; Tuaillon et al. (1994) J. Immunol. 152:2912 2920; Lonberg et al., (1994) Nature 368(6474): 856 859; Lonberg, N. (1994) Handbook of Experimental Pharmacology 113:49 101; Taylor, L. et al. (1994) International Immunology 6: 579 591; Lonberg, N. and Huszar, D. (1995) Intern. Rev. Immunol. Vol. 13: 65 93; Harding, F. and Lonberg, N. (1995) Ann. N.Y. Acad. Sci 764:536 546; Fishwild, D. et al. (1996) Nature Biotechnology 14: 845 851. See further, U.S. Pat. Nos. 5,545,806; 5,569,825; 5,625,126; 5,633,425; 5,789,650; 5,877,397; 5,661,016; 5,814,318; 5,874,299; 5,770,429; and 5,545,807.

In certain embodiments, the antibodies provided herein are able to bind to a receptor or ligand described herein with a dissociation constant of no greater than 10⁻⁶, 10⁻⁷, 10⁻⁸ or 10⁻⁹ M. Standard assays to evaluate the binding ability of the antibodies are known in the art, including for example, ELISAs, Western blots and RIAs. The binding kinetics (e.g., binding affinity) of the antibodies also can be assessed by standard assays known in the art, such as by Biacore analysis. In some embodiments, the binding of the antibody to a receptor described herein substantially inhibits the ability of the corresponding ligand to bind to the receptor. In some embodiments, the binding of the antibody to a ligand described herein substantially inhibits the ability of the ligand to bind to the corresponding receptor. As used herein, an antibody substantially inhibits binding of a receptor and a ligand when an excess of polypeptide reduces the quantity of receptor bound to ligand by at least about 20%, 40%, 60% or 80%, 85% or 90% (as measured in an in vitro competitive binding assay).

c. Small Molecule Agents

Certain embodiments of the methods and compositions disclosed herein relate to methods of inhibiting the activity of or decreasing the levels of a nucleic acid or protein product of a biomarker disclosed herein. Such agents include those disclosed below, those known in the art and those identified using the screening assays described herein.

Agents useful in the methods disclosed herein may be obtained from any available source, including systematic libraries of natural and/or synthetic compounds. Agents may also be obtained by any of the numerous approaches in combinatorial library methods known in the art, including: biological libraries; peptoid libraries (libraries of molecules having the functionalities of peptides, but with a novel, non-peptide backbone which are resistant to enzymatic degradation but which nevertheless remain bioactive; see, e.g., Zuckermann et al., 1994, J. Med. Chem. 37:2678-85); spatially addressable parallel solid phase or solution phase libraries; synthetic library methods requiring deconvolution; the ‘one-bead one-compound’ library method; and synthetic library methods using affinity chromatography selection. The biological library and peptoid library approaches are limited to peptide libraries, while the other four approaches are applicable to peptide, non-peptide oligomer or small molecule libraries of compounds (Lam, 1997, Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. P Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

Libraries of agents may be presented in solution (e.g., Houghten, 1992, Biotechniques 13:412-421), or on beads (Lam, 1991, Nature 354:82-84), chips (Fodor, 1993, Nature 364:555-556), bacteria and/or spores, (Ladner, U.S. Pat. No. 5,223,409), plasmids (Cull et al, 1992, Proc Natl Acad Sci USA 89:1865-1869) or on phage (Scott and Smith, 1990, Science 249:386-390; Devlin, 1990, Science 249:404-406; Cwirla et al, 1990, Proc. Natl. Acad. Sci. 87:6378-6382; Felici, 1991, J. Mol. Biol. 222:301-310; Ladner, supra.).

Agents useful in the methods disclosed herein may be identified, for example, using assays for screening candidate or test compounds which inhibit the activity of or decreases the levels of a nucleic acid or protein product of a biomarker described herein.

d. Interfering Nucleic Acid Agents

In certain embodiments, interfering nucleic acid molecules that selectively target a product of a biomarker provided herein and/or used in methods described herein. Interfering nucleic acids generally include a sequence of cyclic subunits, each bearing a base-pairing moiety, linked by intersubunit linkages that allow the base-pairing moieties to hybridize to a target sequence in a nucleic acid (typically an RNA) by Watson-Crick base pairing, to form a nucleic acid:oligomer heteroduplex within the target sequence. Interfering RNA molecules include, but are not limited to, antisense molecules, siRNA molecules, single-stranded siRNA molecules, miRNA molecules and shRNA molecules.

Typically at least 17, 18, 19, 20, 21, 22 or 23 nucleotides of the complement of the target mRNA sequence are sufficient to mediate inhibition of a target transcript. Perfect complementarity is not necessary. In some embodiments, the interfering nucleic acid molecule is double-stranded RNA. The double-stranded RNA molecule may have a 2 nucleotide 3′ overhang. In some embodiments, the two RNA strands are connected via a hairpin structure, forming a shRNA molecule. shRNA molecules can contain hairpins derived from microRNA molecules. For example, an RNAi vector can be constructed by cloning the interfering RNA sequence into a pCAG-miR30 construct containing the hairpin from the miR30 miRNA. RNA interference molecules may include DNA residues, as well as RNA residues.

Interfering nucleic acid molecules provided herein can contain RNA bases, non-RNA bases or a mixture of RNA bases and non-RNA bases. For example, interfering nucleic acid molecules provided herein can be primarily composed of RNA bases but also contain DNA bases or non-naturally occurring nucleotides.

The interfering nucleic acids can employ a variety of oligonucleotide chemistries. Examples of oligonucleotide chemistries include, without limitation, peptide nucleic acid (PNA), linked nucleic acid (LNA), phosphorothioate, 2′O-Me-modified oligonucleotides, and morpholino chemistries, including combinations of any of the foregoing. In general, PNA and LNA chemistries can utilize shorter targeting sequences because of their relatively high target binding strength relative to 2′O-Me oligonucleotides. Phosphorothioate and 2′O-Me-modified chemistries are often combined to generate 2′O-Me-modified oligonucleotides having a phosphorothioate backbone. See, e.g., PCT Publication Nos. WO/2013/112053 and WO/2009/008725, incorporated by reference in their entireties.

Peptide nucleic acids (PNAs) are analogs of DNA in which the backbone is structurally homomorphous with a deoxyribose backbone, consisting of N-(2-aminoethyl) glycine units to which pyrimidine or purine bases are attached. PNAs containing natural pyrimidine and purine bases hybridize to complementary oligonucleotides obeying Watson-Crick base-pairing rules, and mimic DNA in terms of base pair recognition (Egholm, Buchardt et al. 1993). The backbone of PNAs is formed by peptide bonds rather than phosphodiester bonds, making them well-suited for antisense applications. The backbone is uncharged, resulting in PNA/DNA or PNA/RNA duplexes that exhibit greater than normal thermal stability. PNAs are not recognized by nucleases or proteases.

Despite a radical structural change to the natural structure, PNAs are capable of sequence-specific binding in a helix form to DNA or RNA. Characteristics of PNAs include a high binding affinity to complementary DNA or RNA, a destabilizing effect caused by single-base mismatch, resistance to nucleases and proteases, hybridization with DNA or RNA independent of salt concentration and triplex formation with homopurine DNA. PANAGENE™ has developed its proprietary Bts PNA monomers (Bts; benzothiazole-2-sulfonyl group) and proprietary oligomerization process. The PNA oligomerization using Bts PNA monomers is composed of repetitive cycles of deprotection, coupling and capping. PNAs can be produced synthetically using any technique known in the art. See, e.g., U.S. Pat. Nos. 6,969,766, 7,211,668, 7,022,851, 7,125,994, 7,145,006 and 7,179,896. See also U.S. Pat. Nos. 5,539,082; 5,714,331; and 5,719,262 for the preparation of PNAs. Further teaching of PNA compounds can be found in Nielsen et al., Science, 254:1497-1500, 1991. Each of the foregoing is incorporated by reference in its entirety.

Interfering nucleic acids may also contain “locked nucleic acid” subunits (LNAs). “LNAs” are a member of a class of modifications called bridged nucleic acid (BNA). BNA is characterized by a covalent linkage that locks the conformation of the ribose ring in a C30-endo (northern) sugar pucker. For LNA, the bridge is composed of a methylene between the 2′-O and the 4′-C positions. LNA enhances backbone preorganization and base stacking to increase hybridization and thermal stability.

The structures of LNAs can be found, for example, in Wengel, et al., Chemical Communications (1998) 455; Tetrahedron (1998) 54:3607, and Accounts of Chem. Research (1999) 32:301); Obika, et al., Tetrahedron Letters (1997) 38:8735; (1998) 39:5401, and Bioorganic Medicinal Chemistry (2008) 16:9230. Compounds provided herein may incorporate one or more LNAs; in some cases, the compounds may be entirely composed of LNAs. Methods for the synthesis of individual LNA nucleoside subunits and their incorporation into oligonucleotides are described, for example, in U.S. Pat. Nos. 7,572,582, 7,569,575, 7,084,125, 7,060,809, 7,053,207, 7,034,133, 6,794,499, and 6,670,461, each of which is incorporated by reference in its entirety. Typical intersubunit linkers include phosphodiester and phosphorothioate moieties; alternatively, non-phosphorous containing linkers may be employed. One embodiment is an LNA containing compound where each LNA subunit is separated by a DNA subunit. Certain compounds are composed of alternating LNA and DNA subunits where the intersubunit linker is phosphorothioate.

“Phosphorothioates” (or S-oligos) are a variant of normal DNA in which one of the nonbridging oxygens is replaced by a sulfur. The sulfurization of the internucleotide bond reduces the action of endo-and exonucleases including 5′ to 3′ and 3′ to 5′ DNA POL 1 exonuclease, nucleases S1 and P1, RNases, serum nucleases and snake venom phosphodiesterase. Phosphorothioates are made by two principal routes: by the action of a solution of elemental sulfur in carbon disulfide on a hydrogen phosphonate, or by the method of sulfurizing phosphite triesters with either tetraethylthiuram disulfide (TETD) or 3H-1, 2-bensodithiol-3-one 1, 1-dioxide (BDTD) (see, e.g., Iyer et al., J. Org. Chem. 55, 4693-4699, 1990). The latter methods avoid the problem of elemental sulfur's insolubility in most organic solvents and the toxicity of carbon disulfide. The TETD and BDTD methods also yield higher purity phosphorothioates.

“2′O-Me oligonucleotides” molecules carry a methyl group at the 2′-OH residue of the ribose molecule. 2′-O-Me-RNAs show the same (or similar) behavior as DNA, but are protected against nuclease degradation. 2′-O-Me-RNAs can also be combined with phosphothioate oligonucleotides (PTOs) for further stabilization. 2′O-Me oligonucleotides (phosphodiester or phosphothioate) can be synthesized according to routine techniques in the art (see, e.g., Yoo et al., Nucleic Acids Res. 32:2008-16, 2004).

The interfering nucleic acids described herein may be contacted with a cell or administered to an organism (e.g., a human). Alternatively, constructs and/or vectors encoding the interfering RNA molecules may be contacted with or introduced into a cell or organism. In certain embodiments, a viral, retroviral or lentiviral vector is used. In some embodiments, the vector has a tropism for cardiac tissue. In some embodiments the vector is an adeno-associated virus.

Typically at least 17, 18, 19, 20, 21, 22 or 23 nucleotides of the complement of the target mRNA sequence are sufficient to mediate inhibition of a target transcript. Perfect complementarity is not necessary. In some embodiments, the interfering nucleic acids contains a 1, 2 or 3 nucleotide mismatch with the target sequence. The interfering nucleic acid molecule may have a 2 nucleotide 3′ overhang. If the interfering nucleic acid molecule is expressed in a cell from a construct, for example from a hairpin molecule or from an inverted repeat of the desired sequence, then the endogenous cellular machinery will create the overhangs. shRNA molecules can contain hairpins derived from microRNA molecules. For example, an RNAi vector can be constructed by cloning the interfering RNA sequence into a pCAG-miR30 construct containing the hairpin from the miR30 miRNA. RNA interference molecules may include DNA residues, as well as RNA residues.

In some embodiments, the interfering nucleic acid molecule is a siRNA molecule. Such siRNA molecules should include a region of sufficient homology to the target region, and be of sufficient length in terms of nucleotides, such that the siRNA molecule down-regulate target RNA. The term “ribonucleotide” or “nucleotide” can, in the case of a modified RNA or nucleotide surrogate, also refer to a modified nucleotide, or surrogate replacement moiety at one or more positions. It is not necessary that there be perfect complementarity between the siRNA molecule and the target, but the correspondence must be sufficient to enable the siRNA molecule to direct sequence-specific silencing, such as by RNAi cleavage of the target RNA. In some embodiments, the sense strand need only be sufficiently complementary with the antisense strand to maintain the overall double-strand character of the molecule.

In addition, an siRNA molecule may be modified or include nucleoside surrogates. Single stranded regions of an siRNA molecule may be modified or include nucleoside surrogates, e.g., the unpaired region or regions of a hairpin structure, e.g., a region which links two complementary regions, can have modifications or nucleoside surrogates. Modification to stabilize one or more 3′- or 5′-terminus of an siRNA molecule, e.g., against exonucleases, or to favor the antisense siRNA agent to enter into RISC are also useful. Modifications can include C3 (or C6, C7, C12) amino linkers, thiol linkers, carboxyl linkers, non-nucleotidic spacers (C3, C6, C9, C12, abasic, triethylene glycol, hexaethylene glycol), special biotin or fluorescein reagents that come as phosphoramidites and that have another DMT-protected hydroxyl group, allowing multiple couplings during RNA synthesis.

Each strand of an siRNA molecule can be equal to or less than 35, 30, 25, 24, 23, 22, 21, or 20 nucleotides in length. In some embodiments, the strand is at least 19 nucleotides in length. For example, each strand can be between 21 and 25 nucleotides in length. In some embodiments, siRNA agents have a duplex region of 17, 18, 19, 29, 21, 22, 23, 24, or 25 nucleotide pairs, and one or more overhangs, such as one or two 3′ overhangs, of 2-3 nucleotides.

A “small hairpin RNA” or “short hairpin RNA” or “shRNA” includes a short RNA sequence that makes a tight hairpin turn that can be used to silence gene expression via RNA interference. The shRNAs provided herein may be chemically synthesized or transcribed from a transcriptional cassette in a DNA plasmid. The shRNA hairpin structure is cleaved by the cellular machinery into siRNA, which is then bound to the RNA-induced silencing complex (RISC).

In some embodiments, shRNAs are about 15-60, 15-50, or 15-40 (duplex) nucleotides in length, about 15-30, 15-25, or 19-25 (duplex) nucleotides in length, or are about 20-24, 21-22, or 21-23 (duplex) nucleotides in length (e.g., each complementary sequence of the double-stranded shRNA is 15-60, 15-50, 15-40, 15-30, 15-25, or 19-25 nucleotides in length, or about 20-24, 21-22, or 21-23 nucleotides in length, and the double-stranded shRNA is about 15-60, 15-50, 15-40, 15-30, 15-25, or 19-25 base pairs in length, or about 18-22, 19-20, or 19-21 base pairs in length). shRNA duplexes may comprise 3′ overhangs of about 1 to about 4 nucleotides or about 2 to about 3 nucleotides on the antisense strand and/or 5′-phosphate termini on the sense strand. In some embodiments, the shRNA comprises a sense strand and/or antisense strand sequence of from about 15 to about 60 nucleotides in length (e.g., about 15-60, 15-55, 15-50, 15-45, 15-40, 15-35, 15-30, or 15-25 nucleotides in length), or from about 19 to about 40 nucleotides in length (e.g., about 19-40, 19-35, 19-30, or 19-25 nucleotides in length), or from about 19 to about 23 nucleotides in length (e.g., 19, 20, 21, 22, or 23 nucleotides in length).

Non-limiting examples of shRNA include a double-stranded polynucleotide molecule assembled from a single-stranded molecule, where the sense and antisense regions are linked by a nucleic acid-based or non-nucleic acid-based linker; and a double-stranded polynucleotide molecule with a hairpin secondary structure having self-complementary sense and antisense regions. In some embodiments, the sense and antisense strands of the shRNA are linked by a loop structure comprising from about 1 to about 25 nucleotides, from about 2 to about 20 nucleotides, from about 4 to about 15 nucleotides, from about 5 to about 12 nucleotides, or 1, 2, 3, 4, 5, 6, 7, 8, 9, 10, 11, 12, 13, 14, 15, 16, 17, 18, 19, 20, 21, 22, 23, 24, 25, or more nucleotides.

Additional embodiments related to the shRNAs, as well as methods of designing and synthesizing such shRNAs, are described in U.S. patent application publication number 2011/0071208, the disclosure of which is herein incorporated by reference in its entirety for all purposes.

In some embodiments, provided herein are micro RNAs (miRNAs). miRNAs represent a large group of small RNAs produced naturally in organisms, some of which regulate the expression of target genes. miRNAs are formed from an approximately 70 nucleotide single-stranded hairpin precursor transcript by Dicer. miRNAs are not translated into proteins, but instead bind to specific messenger RNAs, thereby blocking translation. In some instances, miRNAs base-pair imprecisely with their targets to inhibit translation.

In some embodiments, antisense oligonucleotide compounds are provided herein. In certain embodiments, the degree of complementarity between the target sequence and antisense targeting sequence is sufficient to form a stable duplex. The region of complementarity of the antisense oligonucleotides with the target RNA sequence may be as short as 8-11 bases, but can be 12-15 bases or more, e.g., 10-40 bases, 12-30 bases, 12-25 bases, 15-25 bases, 12-20 bases, or 15-20 bases, including all integers in between these ranges. An antisense oligonucleotide of about 14-15 bases is generally long enough to have a unique complementary sequence.

In certain embodiments, antisense oligonucleotides may be 100% complementary to the target sequence, or may include mismatches, e.g., to improve selective targeting of allele containing the disease-associated mutation, as long as a heteroduplex formed between the oligonucleotide and target sequence is sufficiently stable to withstand the action of cellular nucleases and other modes of degradation which may occur in vivo. Hence, certain oligonucleotides may have about or at least about 70% sequence complementarity, e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence complementarity, between the oligonucleotide and the target sequence. Oligonucleotide backbones that are less susceptible to cleavage by nucleases are discussed herein. Mismatches, if present, are typically less destabilizing toward the end regions of the hybrid duplex than in the middle. The number of mismatches allowed will depend on the length of the oligonucleotide, the percentage of G:C base pairs in the duplex, and the position of the mismatch(es) in the duplex, according to well understood principles of duplex stability.

Interfering nucleic acid molecules can be prepared, for example, by chemical synthesis, in vitro transcription, or digestion of long dsRNA by Rnase III or Dicer. These can be introduced into cells by transfection, electroporation, or other methods known in the art. See Hannon, G J, 2002, RNA Interference, Nature 418: 244-251; Bernstein E et al., 2002, The rest is silence. RNA 7: 1509-1521; Hutvagner G et al., RNAi: Nature abhors a double-strand. Curr. Opin. Genetics & Development 12: 225-232; Brummelkamp, 2002, A system for stable expression of short interfering RNAs in mammalian cells. Science 296: 550-553; Lee N S, Dohjima T, Bauer G, Li H, Li M-J, Ehsani A, Salvaterra P, and Rossi J. (2002). Expression of small interfering RNAs targeted against HIV-1 rev transcripts in human cells. Nature Biotechnol. 20:500-505; Miyagishi M, and Taira K. (2002). U6-promoter-driven siRNAs with four uridine 3′ overhangs efficiently suppress targeted gene expression in mammalian cells. Nature Biotechnol. 20:497-500; Paddison P J, Caudy A A, Bernstein E, Hannon G J, and Conklin D S. (2002). Short hairpin RNAs (shRNAs) induce sequence-specific silencing in mammalian cells. Genes & Dev. 16:948-958; Paul C P, Good P D, Winer I, and Engelke D R. (2002). Effective expression of small interfering RNA in human cells. Nature Biotechnol. 20:505-508; Sui G, Soohoo C, Affar E-B, Gay F, Shi Y, Forrester W C, and Shi Y. (2002). A DNA vector-based RNAi technology to suppress gene expression in mammalian cells. Proc. Natl. Acad. Sci. USA 99(6):5515-5520; Yu J-Y, DeRuiter S L, and Turner D L. (2002). RNA interference by expression of short-interfering RNAs and hairpin RNAs in mammalian cells. Proc. Natl. Acad. Sci. USA 99(9):6047-6052.

In the present methods, an interfering nucleic acid molecule or an interfering nucleic acid encoding polynucleotide can be administered to the subject, for example, as naked nucleic acid, in combination with a delivery reagent, and/or as a nucleic acid comprising sequences that express an interfering nucleic acid molecule. In some embodiments the nucleic acid comprising sequences that express the interfering nucleic acid molecules are delivered within vectors, e.g. plasmid, viral and bacterial vectors. Any nucleic acid delivery method known in the art can be used in the methods described herein. Suitable delivery reagents include, but are not limited to, e.g., the Mirus Transit TKO lipophilic reagent; lipofectin; lipofectamine; cellfectin; polycations (e.g., polylysine), atelocollagen, nanoplexes and liposomes. The use of atelocollagen as a delivery vehicle for nucleic acid molecules is described in Minakuchi et al. Nucleic Acids Res., 32(13):e109 (2004); Hanai et al. Ann NY Acad Sci., 1082:9-17 (2006); and Kawata et al. Mol Cancer Ther., 7(9):2904-12 (2008); each of which is incorporated herein in their entirety. Exemplary interfering nucleic acid delivery systems are provided in U.S. Pat. Nos. 8,283,461, 8,313,772, 8,501,930, 8,426,554, 8,268,798 and 8,324,366, each of which is hereby incorporated by reference in its entirety.

In some embodiments of the methods described herein, liposomes are used to deliver an inhibitory oligonucleotide to a subject. Liposomes suitable for use in the methods described herein can be formed from standard vesicle-forming lipids, which generally include neutral or negatively charged phospholipids and a sterol, such as cholesterol. The selection of lipids is generally guided by consideration of factors such as the desired liposome size and half-life of the liposomes in the blood stream. A variety of methods are known for preparing liposomes, for example, as described in Szoka et al. (1980), Ann. Rev. Biophys. Bioeng. 9:467; and U.S. Pat. Nos. 4,235,871, 4,501,728, 4,837,028, and 5,019,369, the entire disclosures of which are herein incorporated by reference.

The liposomes for use in the present methods can also be modified so as to avoid clearance by the mononuclear macrophage system (“MMS”) and reticuloendothelial system (“RES”). Such modified liposomes have opsonization-inhibition moieties on the surface or incorporated into the liposome structure.

Opsonization-inhibiting moieties for use in preparing the liposomes described herein are typically large hydrophilic polymers that are bound to the liposome membrane. As used herein, an opsonization inhibiting moiety is “bound” to a liposome membrane when it is chemically or physically attached to the membrane, e.g., by the intercalation of a lipid-soluble anchor into the membrane itself, or by binding directly to active groups of membrane lipids. These opsonization-inhibiting hydrophilic polymers form a protective surface layer that significantly decreases the uptake of the liposomes by the MMS and RES; e.g., as described in U.S. Pat. No. 4,920,016, the entire disclosure of which is herein incorporated by reference.

In some embodiments, opsonization inhibiting moieties suitable for modifying liposomes are water-soluble polymers with a number-average molecular weight from about 500 to about 40,000 daltons, or from about 2,000 to about 20,000 daltons. Such polymers include polyethylene glycol (PEG) or polypropylene glycol (PPG) derivatives; e.g., methoxy PEG or PPG, and PEG or PPG stearate; synthetic polymers such as polyacrylamide or poly N-vinyl pyrrolidone; linear, branched, or dendrimeric polyamidoamines; polyacrylic acids; polyalcohols, e.g., polyvinylalcohol and polyxylitol to which carboxylic or amino groups are chemically linked, as well as gangliosides, such as ganglioside GM1. Copolymers of PEG, methoxy PEG, or methoxy PPG, or derivatives thereof, are also suitable. In addition, the opsonization inhibiting polymer can be a block copolymer of PEG and either a polyamino acid, polysaccharide, polyamidoamine, polyethyleneamine, or polynucleotide. The opsonization inhibiting polymers can also be natural polysaccharides containing amino acids or carboxylic acids, e.g., galacturonic acid, glucuronic acid, mannuronic acid, hyaluronic acid, pectic acid, neuraminic acid, alginic acid, carrageenan; aminated polysaccharides or oligosaccharides (linear or branched); or carboxylated polysaccharides or oligosaccharides, e.g., reacted with derivatives of carbonic acids with resultant linking of carboxylic groups. In some embodiments, the opsonization-inhibiting moiety is a PEG, PPG, or derivatives thereof. Liposomes modified with PEG or PEG-derivatives are sometimes called “PEGylated liposomes.”

### Screening Methods

In one embodiment, the present invention relates to assays for screening test agents which bind to, or modulate the biological activity of, at least one biomarker described herein. In one embodiment, a method for identifying such an agent entails determining the ability of the agent to modulate, e.g. inhibit, the at least one biomarker described herein.

In one embodiment, an assay is a cell-free or cell-based assay, comprising contacting at least one biomarker described herein, with a test agent, and determining the ability of the test agent to modulate (e.g., inhibit) the activity of the biomarker (e.g., the activity of a gene product, such as a polypeptide, encoded by a gene disclosed herein), such as by measuring direct binding of substrates or by measuring indirect parameters as described below.

For example, in a direct binding assay, biomarker protein (or their respective target polypeptides or molecules) can be coupled with a radioisotope or enzymatic label such that binding can be determined by detecting the labeled protein or molecule in a complex. For example, the targets can be labeled with ¹²⁵I, ³⁵S, ¹⁴C, or ³H, either directly or indirectly, and the radioisotope detected by direct counting of radioemmission or by scintillation counting. Alternatively, the targets can be enzymatically labeled with, for example, horseradish peroxidase, alkaline phosphatase, or luciferase, and the enzymatic label detected by determination of conversion of an appropriate substrate to product. Determining the interaction between biomarker and substrate can also be accomplished using standard binding or enzymatic analysis assays. In one or more embodiments of the above described assay methods, it may be desirable to immobilize polypeptides or molecules to facilitate separation of complexed from uncomplexed forms of one or both of the proteins or molecules, as well as to accommodate automation of the assay.

Binding of a test agent to a target can be accomplished in any vessel suitable for containing the reactants. Non-limiting examples of such vessels include microtiter plates, test tubes, and micro-centrifuge tubes. Immobilized forms of the antibodies described herein can also include antibodies bound to a solid phase like a porous, microporous (with an average pore diameter less than about one micron) or macroporous (with an average pore diameter of more than about 10 microns) material, such as a membrane, cellulose, nitrocellulose, or glass fibers; a bead, such as that made of agarose or polyacrylamide or latex; or a surface of a dish, plate, or well, such as one made of polystyrene.

In an alternative embodiment, determining the ability of the agent to modulate the interaction between the biomarker and a substrate or a biomarker and its natural binding partner can be accomplished by determining the ability of the test agent to modulate the activity of a polypeptide or other product that functions downstream or upstream of its position within the signaling pathway (e.g., feedback loops). Such feedback loops are well-known in the art (see, for example, Chen and Guillemin (2009) Int. J. Tryptophan Res. 2:1-19).

The present invention further pertains to novel agents identified by the above-described screening assays. Accordingly, it is within the scope of this invention to further use an agent identified as described herein, such as in an appropriate animal model. For example, an agent identified as described herein can be used in an animal model to determine the efficacy, toxicity, or side effects of treatment with such an agent. Alternatively, an antibody identified as described herein can be used in an animal model to determine the mechanism of action of such an agent.

### Predictive Medicine

The present invention also pertains to the field of predictive medicine in which diagnostic assays, prognostic assays, and monitoring clinical trials are used for prognostic (predictive) purposes to thereby treat an individual prophylactically. Accordingly, one aspect of the present invention relates to diagnostic assays for determining the amount and/or activity level of a gene product of a biomarker disclosed herein in the context of a biological sample (e.g., blood, serum, cells, or tissue) to thereby determine whether an individual is at risk for an endometriosis related condition. Such assays can be used for prognostic or predictive purpose to thereby prophylactically treat an individual prior to the onset or after recurrence of a disorder characterized by or associated with biomarker polypeptide, nucleic acid expression or activity. The skilled artisan will appreciate that any method can use one or more (e.g., combinations) of biomarkers listed herein.

Another aspect of the present invention pertains to monitoring the influence of agents (e.g., drugs, compounds, and small nucleic acid-based molecules) on the expression or activity of a biomarker disclosed herein.

The skilled artisan will also appreciated that, in certain embodiments, the methods of the present invention implement a computer program and computer system. For example, a computer program can be used to perform the algorithms described herein. A computer system can also store and manipulate data generated by the methods of the present invention which comprises a plurality of biomarker signal changes/profiles which can be used by a computer system in implementing the methods of this invention. In certain embodiments, a computer system receives biomarker expression data; (ii) stores the data; and (iii) compares the data in any number of ways described herein (e.g., analysis relative to appropriate controls) to determine the state of informative biomarkers from cancerous or pre-cancerous tissue. In other embodiments, a computer system (i) compares the determined expression biomarker level to a threshold value; and (ii) outputs an indication of whether said biomarker level is significantly modulated (e.g., above or below) the threshold value, or a phenotype based on said indication.

In certain embodiments, such computer systems are also considered part of the present invention. Numerous types of computer systems can be used to implement the analytic methods of this invention according to knowledge possessed by a skilled artisan in the bioinformatics and/or computer arts. Several software components can be loaded into memory during operation of such a computer system. The software components can comprise both software components that are standard in the art and components that are special to the present invention (e.g., dCHIP software described in Lin et al. (2004) Bioinformatics 20, 1233-1240; radial basis machine learning algorithms (RBM) known in the art).

The methods of the present invention can also be programmed or modeled in mathematical software packages that allow symbolic entry of equations and high-level specification of processing, including specific algorithms to be used, thereby freeing a user of the need to procedurally program individual equations and algorithms. Such packages include, e.g., Matlab from Mathworks (Natick, Mass.), Mathematica from Wolfram Research (Champaign, 111.) or S-Plus from Math Soft (Seattle, Wash.).

In certain embodiments, the computer comprises a database for storage of biomarker data. Such stored profiles can be accessed and used to perform comparisons of interest at a later point in time. For example, biomarker expression profiles of a sample derived from the non-cancerous tissue of a subject and/or profiles generated from population-based distributions of informative loci of interest in relevant populations of the same species can be stored and later compared to that of a sample derived from the cancerous tissue of the subject or tissue suspected of being cancerous of the subject.

In addition to the exemplary program structures and computer systems described herein, other, alternative program structures and computer systems will be readily apparent to the skilled artisan. Such alternative systems, which do not depart from the above described computer system and programs structures either in spirit or in scope, are therefore intended to be comprehended within the accompanying claims.

### Pharmaceutical Compositions

In another aspect, the present invention provides pharmaceutically acceptable compositions which comprise a therapeutically-effective amount of an agent that modulates (e.g., decreases) biomarker expression and/or activity, formulated together with one or more pharmaceutically acceptable carriers (additives) and/or diluents. As described in detail below, the pharmaceutical compositions of the present invention may be specially formulated for administration in solid or liquid form, including those adapted for the following: (1) oral administration, for example, drenches (aqueous or non-aqueous solutions or suspensions), tablets, boluses, powders, granules, pastes; (2) parenteral administration, for example, by subcutaneous, intramuscular or intravenous injection as, for example, a sterile solution or suspension; (3) topical application, for example, as a cream, ointment or spray applied to the skin; (4) intravaginally or intrarectally, for example, as a pessary, cream or foam; or (5) aerosol, for example, as an aqueous aerosol, liposomal preparation or solid particles containing the compound.

The phrase “therapeutically-effective amount” as used herein means that amount of an agent that modulates (e.g., inhibits) biomarker expression and/or activity, or expression and/or activity of the complex, or composition comprising an agent that modulates (e.g., inhibits) biomarker expression and/or activity, or expression and/or activity of the complex, which is effective for producing some desired therapeutic effect, e.g., cancer treatment, at a reasonable benefit/risk ratio.

The phrase “pharmaceutically acceptable” is employed herein to refer to those agents, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, solvent or encapsulating material, involved in carrying or transporting the subject chemical from one organ, or portion of the body, to another organ, or portion of the body. Each carrier must be “acceptable” in the sense of being compatible with the other ingredients of the formulation and not injurious to the subject. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water (17) isotonic saline; (18) Ringer's solution; (19) ethyl alcohol; (20) phosphate buffer solutions; and (21) other non-toxic compatible substances employed in pharmaceutical formulations.

The term “pharmaceutically-acceptable salts” refers to the relatively non-toxic, inorganic and organic acid addition salts of the agents that modulates (e.g., inhibits) biomarker expression and/or activity, or expression and/or activity of the complex encompassed by the present invention. These salts can be prepared in situ during the final isolation and purification of the therapeutic agents, or by separately reacting a purified therapeutic agent in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like (See, for example, Berge et al. (1977) “Pharmaceutical Salts”, J. Pharm. Sci. 66:1-19).

In other cases, the agents useful in the methods of the present invention may contain one or more acidic functional groups and, thus, are capable of forming pharmaceutically-acceptable salts with pharmaceutically-acceptable bases. The term “pharmaceutically-acceptable salts” in these instances refers to the relatively non-toxic, inorganic and organic base addition salts of agents that modulates (e.g., inhibits) biomarker expression and/or activity, or expression and/or activity of the complex. These salts can likewise be prepared in situ during the final isolation and purification of the therapeutic agents, or by separately reacting the purified therapeutic agent in its free acid form with a suitable base, such as the hydroxide, carbonate or bicarbonate of a pharmaceutically-acceptable metal cation, with ammonia, or with a pharmaceutically-acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like (see, for example, Berge et al., supra).

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations useful in the methods of the present invention include those suitable for oral, nasal, topical (including buccal and sublingual), rectal, vaginal, aerosol and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient, which can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an agent that modulates (e.g., inhibits) biomarker expression and/or activity, with the carrier and, optionally, one or more accessory ingredients. In general, the formulations are prepared by uniformly and intimately bringing into association a therapeutic agent with liquid carriers, or finely divided solid carriers, or both, and then, if necessary, shaping the product.

Formulations suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a therapeutic agent as an active ingredient. A compound may also be administered as a bolus, electuary or paste.

In solid dosage forms for oral administration (capsules, tablets, pills, dragees, powders, granules and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered peptide or peptidomimetic moistened with an inert liquid diluent.

Tablets, and other solid dosage forms, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions, which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions, which can be used include polymeric substances and waxes. The active ingredient can also be in micro-encapsulated form, if appropriate, with one or more of the above-described excipients.

Liquid dosage forms for oral administration include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active agent may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations for rectal or vaginal administration may be presented as a suppository, which may be prepared by mixing one or more therapeutic agents with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active agent.

Formulations which are suitable for vaginal administration also include pessaries, tampons, creams, gels, pastes, foams or spray formulations containing such carriers as are known in the art to be appropriate.

Dosage forms for the topical or transdermal administration of an agent that modulates (e.g., inhibits) biomarker expression and/or activity include powders, sprays, ointments, pastes, creams, lotions, gels, solutions, patches and inhalants. The active component may be mixed under sterile conditions with a pharmaceutically-acceptable carrier, and with any preservatives, buffers, or propellants which may be required.

The ointments, pastes, creams and gels may contain, in addition to a therapeutic agent, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an agent that modulates (e.g., inhibits) biomarker expression and/or activity, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

The agent that modulates (e.g., inhibits) biomarker expression and/or activity, can be alternatively administered by aerosol. This is accomplished by preparing an aqueous aerosol, liposomal preparation or solid particles containing the compound. A nonaqueous (e.g., fluorocarbon propellant) suspension could be used. Sonic nebulizers are preferred because they minimize exposing the agent to shear, which can result in degradation of the compound.

Ordinarily, an aqueous aerosol is made by formulating an aqueous solution or suspension of the agent together with conventional pharmaceutically acceptable carriers and stabilizers. The carriers and stabilizers vary with the requirements of the particular compound, but typically include nonionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Transdermal patches have the added advantage of providing controlled delivery of a therapeutic agent to the body. Such dosage forms can be made by dissolving or dispersing the agent in the proper medium. Absorption enhancers can also be used to increase the flux of the peptidomimetic across the skin. The rate of such flux can be controlled by either providing a rate controlling membrane or dispersing the peptidomimetic in a polymer matrix or gel.

Pharmaceutical compositions of this invention suitable for parenteral administration comprise one or more therapeutic agents in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the present invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of an agent that modulates (e.g., inhibits) biomarker expression and/or activity, in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions, which are compatible with body tissue.

When the therapeutic agents of the present invention are administered as pharmaceuticals, to humans and animals, they can be given per se or as a pharmaceutical composition containing, for example, 0.1 to 99.5% (more preferably, 0.5 to 90%) of active ingredient in combination with a pharmaceutically acceptable carrier.

Actual dosage levels of the active ingredients in the pharmaceutical compositions of this invention may be determined by the methods of the present invention so as to obtain an amount of the active ingredient, which is effective to achieve the desired therapeutic response for a particular subject, composition, and mode of administration, without being toxic to the subject.

Conventional dosage forms generally provide rapid or immediate drug release from the formulation. Depending on the pharmacology and pharmacokinetics of the drug, use of conventional dosage forms can lead to wide fluctuations in the concentrations of the drug in a patient's blood and other tissues. These fluctuations can impact a number of parameters, such as dose frequency, onset of action, duration of efficacy, maintenance of therapeutic blood levels, toxicity, side effects, and the like. Advantageously, controlled-release formulations can be used to control a drug's onset of action, duration of action, plasma levels within the therapeutic window, and peak blood levels. In particular, controlled- or extended-release dosage forms or formulations can be used to ensure that the maximum effectiveness of a drug is achieved while minimizing potential adverse effects and safety concerns, which can occur both from under-dosing a drug (i.e., going below the minimum therapeutic levels) as well as exceeding the toxicity level for the drug. In some embodiments, the biomarker inhibitor and/or binding reagent can be administered in a sustained release formulation.

Controlled-release pharmaceutical products have a common goal of improving drug therapy over that achieved by their non-controlled release counterparts. Ideally, the use of an optimally designed controlled-release preparation in medical treatment is characterized by a minimum of drug substance being employed to cure or control the condition in a minimum amount of time. Advantages of controlled-release formulations include: 1) extended activity of the drug; 2) reduced dosage frequency; 3) increased patient compliance; 4) usage of less total drug: 5) reduction in local or systemic side effects: 6) minimization of drug accumulation; 7) reduction in blood level fluctuations; 8) improvement in efficacy of treatment; 9) reduction of potentiation or loss of drug activity: and 10) improvement in speed of control of diseases or conditions. Kim, Cherng-ju, Controlled Release Dosage Form Design, 2 (Technomic Publishing. Lancaster, Pa.: 2000).

The nucleic acid molecules of the present invention can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. (1994) Proc. Natl. Acad. Sci. USA 91:3054 3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

The present invention also encompasses kits for detecting and/or modulating biomarkers described herein. A kit of the present invention may also include instructional materials disclosing or describing the use of the kit or an antibody of the disclosed invention in a method of the disclosed invention as provided herein. A kit may also include additional components to facilitate the particular application for which the kit is designed. For example, a kit may additionally contain means of detecting the label (e.g., enzyme substrates for enzymatic labels, filter sets to detect fluorescent labels, appropriate secondary labels such as a sheep anti-mouse-HRP, etc.) and reagents necessary for controls (e.g., control biological samples or standards). A kit may additionally include buffers and other reagents recognized for use in a method of the disclosed invention. Non-limiting examples include agents to reduce non-specific binding, such as a carrier protein or a detergent.

### Kits

The present invention also encompasses kits for detecting and/or modulating biomarkers described herein. A kit of the present invention may also include instructional materials disclosing or describing the use of the kit or an antibody of the disclosed invention in a method of the disclosed invention as provided herein. A kit may also include additional components to facilitate the particular application for which the kit is designed. For example, a kit may additionally contain means of detecting the label (e.g., enzyme substrates for enzymatic labels, filter sets to detect fluorescent labels, appropriate secondary labels such as a sheep anti-mouse-HRP, etc.) and reagents necessary for controls (e.g., control biological samples or standards). A kit may additionally include buffers and other reagents recognized for use in a method of the disclosed invention. Non-limiting examples include agents to reduce non-specific binding, such as a carrier protein or a detergent.

### Examples

The standard diagnostic method for endometriosis is surgical pelvic laparoscopy and combined with nonspecific symptoms has resulted in an average eight-year gap between the onset of disease to time of diagnosis. The protein profile, identified by the Applicant, associated with endometriosis may serve as a noninvasive diagnostic tool for early detection of endometriosis.

Several papers have identified primarily microRNA's as potential markers of the disease but there is limited cross validation between them. These have been done primarily with serum or plasma and do not take into account the different types of endometriosis and how they contribute to the changes in the microRNA profile. Using a baboon model in which samples from the same animal are obtained prior to and following the induction of the disease, it was confirmed that the animal does not have spontaneous disease. This data provides direct evidence that it is the presence of the endometriotic lesions that impacts the changes in the proteome. The baboon model has peritoneal disease, and the fact that these changes are reflected in women suggests that this would be an early diagnostic since peritoneal disease is the most common form seen in adolescents and younger women.

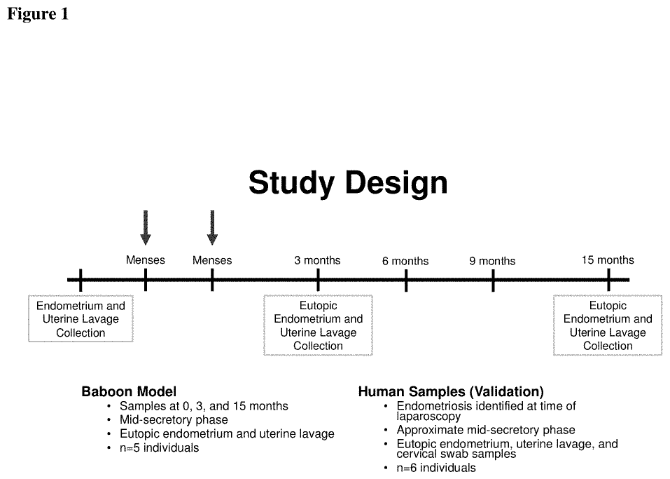

Uterine secretions, or histotroph, contain an amalgam of proteins, lipids, amino acids, ions, and sugars which are dynamic throughout the menstrual cycle and pregnancy. Uterine proteins may be useful for the diagnosis of endometriosis since the presence of ectopic lesions alters gene expression and progesterone responsiveness of the uterine endometrium. Studies to date have focused on identifying biomarkers of endometriosis from endometrial tissue, peritoneal fluid, menstrual fluid, and serum and plasma but have ultimately been unsuccessful in validating their results in large cohorts of patients due to the heterogeneity of the disease. The experimentation herein investigates the development of a biomarker panel from uterine fluids as a noninvasive diagnostic for endometriosis. Utilizing a baboon, Papio anubis, model of induced endometriosis and high-throughput proteomic analyses of longitudinal uterine lavages, a profile of differentially expressed proteins (DEPs) throughout the course of disease has been identified (FIG. 1).

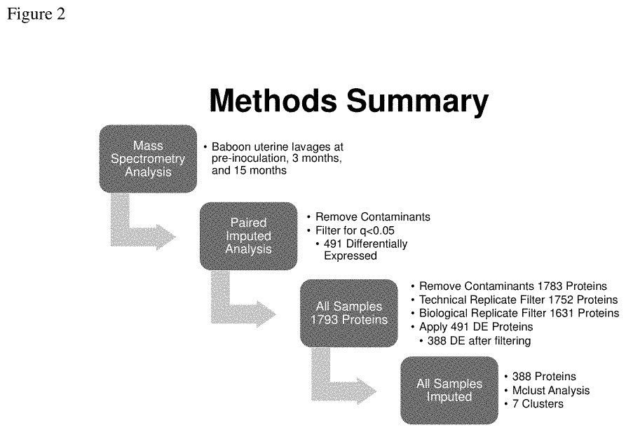

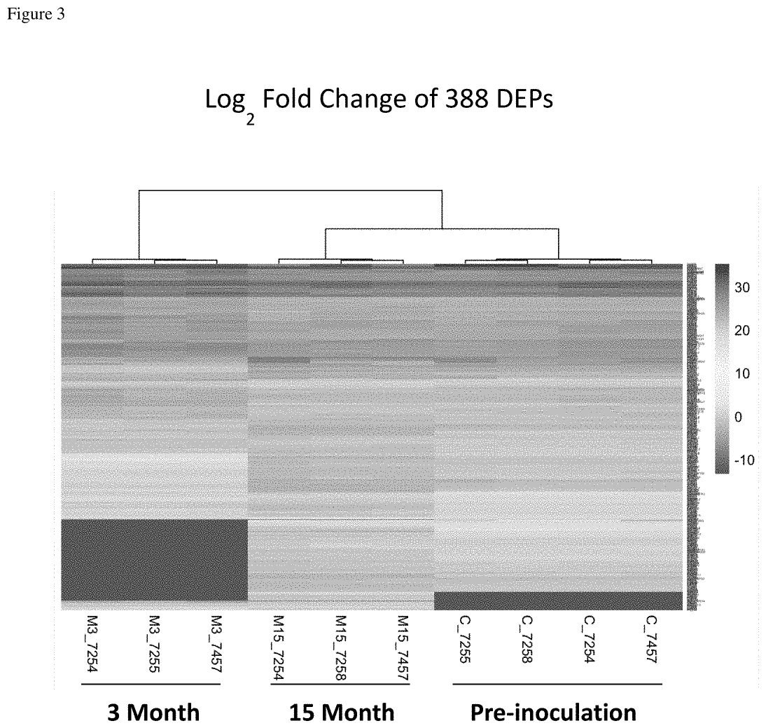

Control or pre-inoculation, three-month, and fifteen-month endometriosis uterine lavages from a total of five animals were investigated to discern differentially expressed proteins throughout the course of disease and found 388 proteins that were significantly differentially expressed (FIG. 2, FIG. 3). Equal protein concentration (estimated by densitometric analysis) of 3 samples from control and each disease stage were subjected to label free mass spectrometry protein quantification. Briefly, proteins were digested in 50% acetonitrile containing LysC and Trypsin overnight at 37° C. The digested samples were dried and reconstituted in 25 mM ammonium bicarbonate/4% acetonitrile. Samples were loaded on to a C18 column (2 μm particles, 25 cm×75 μm ID) and eluted using a 2 hr acetonitrile gradient into a Q-Exactive HF-X mass spectrometer. Each sample was run three times to account for technical variability. A total of 388 proteins were identified as significantly differentially expressed.

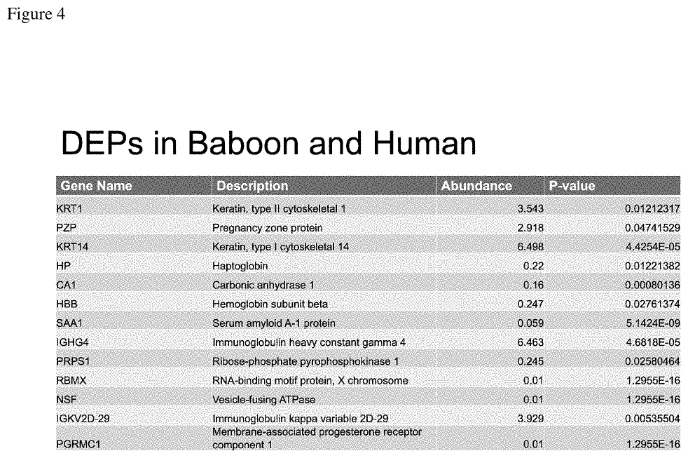

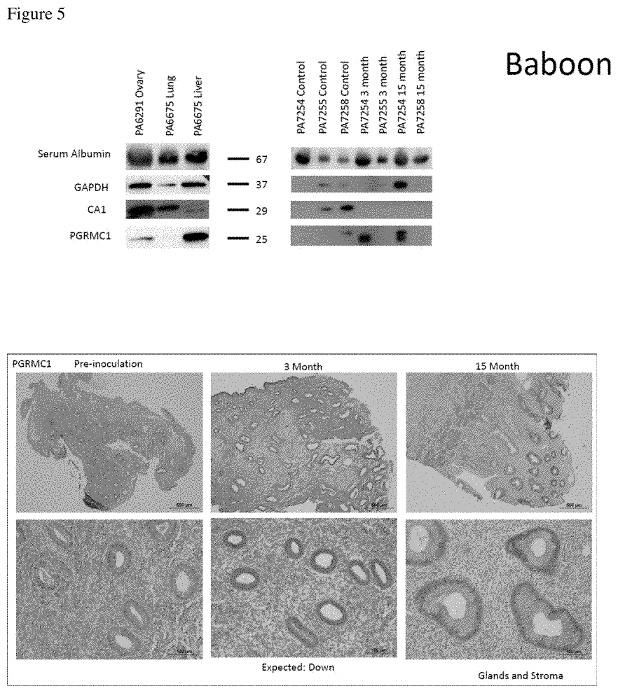

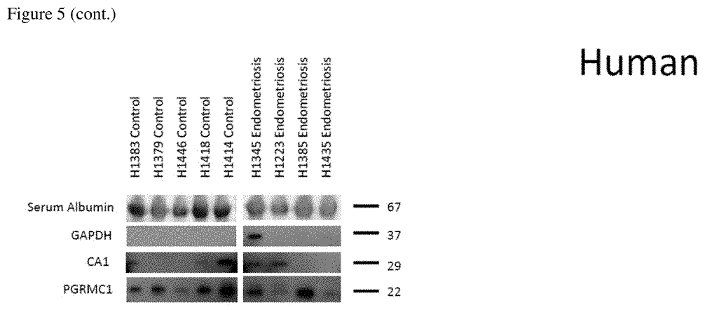

Independent analysis of human uterine lavage samples from women with and without disease (n=6) was then performed to determine a differentially expressed proteomic profile in humans. Thirteen proteins were commonly differentially expressed in the baboon model and human patients with endometriosis including KRT1, PZP, KRT14, HP, CA1, HBB, SAA1, IGHG4, PRPS1, RBMX, NSF, IGKV2D-29, and PGRMC1 (FIG. 4). Validation of the proteomic profile of disease from the baboon model will be carried out in a large cohort of women with endometriosis to develop a biomarker panel of disease.

The Applicant has established a baboon, Papio anubis, model of induced endometriosis to study the molecular mechanisms associated with this gynecological disease to develop better diagnostic and treatment strategies.

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

Also incorporated by reference in their entirety are any polynucleotide and polypeptide sequences which reference an accession number correlating to an entry in a public database, such as those maintained by The Institute for Genomic Research (TIGR) on the world wide web at tigr.org and/or the National Center for Biotechnology Information (NCBI) on the World Wide Web at ncbi.nlm.nih.gov.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A method of determining whether a subject has an endometriosis-related condition, the method comprising
measuring the levels of or activity of at least one protein encoded by at least one gene selected from KRT1, PZP, KRT14, HP, CA1, HBB, SAA1, IGHG4, PRPS1, RBMX, NSF, IGKV2D-29, and PGRMC1 in a biological sample isolated from the subject, and
if the level of or activity of the protein(s) is elevated compared to a level or activity of the protein(s) in a biological sample from a subject who is not afflicted with an endometriosis-related condition, the subject is determined to have the endometrioses-related condition.

2. The method of claim 1, wherein the method comprises measuring the levels of or the activity of at least two proteins encoded by at least two genes, at least three genes, at least four genes, at least five genes, at least six genes, at least seven genes, at least eight genes, at least nine genes, at least ten genes, at least eleven genes, at least twelve genes, or at least thirteen genes selected from KRT1, PZP, KRT14, HP, CA1, HBB, SAA1, IGHG4, PRPS1, RBMX, NSF, IGKV2D-29, and PGRMC1.

3-13. (canceled)

14. The method of claim 1, wherein the levels or activity of the at least one protein(s) is measured by contacting the biological sample with an antibody specific for the at least one protein(s).

15. The method of claim 1, wherein if the subject is determined to have an endometriosis-related condition, the method further comprises administering to the subject a therapy for the endometriosis-related condition.

16. A method of determining whether a subject has an endometriosis-related condition, the method comprising,
measuring the levels of at least one RNA product of at least one gene selected from KRT1, PZP, KRT14, HP, CA1, HBB, SAA1, IGHG4, PRPS1, RBMX, NSF, IGKV2D-29, and PGRMC1 in a biological sample isolated from the subject, and
if the level of the at least one RNA product is elevated compared to a level of the RNA product in a subject who is not afflicted with an endometrioses-related condition, the subject is determined to have an endometrioses-related condition.

17. The method of claim 16, wherein the method comprises measuring the levels of at least two RNA products of at least two genes, at least three genes, at least four genes, at least five genes, at least six genes, at least seven genes, at least eight genes, at least nine genes, at least ten genes, at least eleven genes, at least twelve genes, or at least thirteen genes selected from KRT1, PZP, KRT14, HP, CA1, HBB, SAA1, IGHG4, PRPS1, RBMX, NSF, IGKV2D-29, and PGRMC1.

18-29. (canceled)

30. A method of treating or preventing an endometriosis-related condition in a subject in need thereof, the method comprising
measuring the levels of or activity of at least one protein encoded by or the levels of an at least one RNA product of at least one gene selected from KRT1, PZP, KRT14, HP, CA1, HBB, SAA1, IGHG4, PRPS1, RBMX, NSF, IGKV2D-29, and PGRMC1 in a biological sample isolated from the subject, and
if the level of or activity of the at least one protein(s) or if the level of at least one RNA product(s) is elevated compared to a level or activity of the at least one protein(s) or the level of at least one RNA product(s) in a biological sample from a subject who is not afflicted with an endometrioses-related condition, administering to the subject a therapy for the endometriosis-related condition.

31. (canceled)

32. The method of claim 30, wherein the therapy is a hormonal therapy.

33. The method of claim 30, wherein the biological sample is endometrial tissue.

34. The method of claim 30, wherein the biological sample comprises cervical cells.

35. The method of claim 30, wherein the biological sample is uterine tissue.

36. The method of claim 30, wherein the endometriosis-related condition is endometriosis, endometriotic cysts, endometrioid cancer, or ovarian cancer.

37-39. (canceled)
